SEQUENCE LISTING (2 sequences)

SEQ ID NO: 1 - protein, 4 aa, synthetic construct
GFLG

SEQ ID NO: 2 - protein, 4 aa, synthetic construct
ALAL

(19) United States
(12) Patent Application Publication
(10) Pub. No.: US 2025/0009890 A1
(43) Pub. Date: Jan. 9, 2025

(54) TRANS-CYCLOOCTENE-MODIFIED BISPECIFIC ANTIBODIES

(71) Applicant: Tambo, Inc., San Francisco, CA (US)

(72) Inventors: José Manuel MEJÍA ONETO, San Francisco, CA (US); Michael ZAKHARIAN, Los Angeles, CA (US); Jesse M. MCFARLAND, Berkeley, CA (US); Amir MAHMOODI, Fairfield, CA (US)

(21) Appl. No.: 18/707,534

(22) PCT Filed: Nov. 4, 2022
(86) PCT No.: PCT/US2022/079285

§ 371 Date: May 3, 2024

Related U.S. Application Data
(60) Provisional application No. 63/276,401, filed on Nov. 5, 2021.

(51) Int. Cl. A61K 47/54; C07K 16/28

(52) U.S. Cl. CPC A61K 47/545; C07K 16/2809; C07K 2317/31; C07K 2317/55; C07K 2317/569; C07K 2317/622

(57) ABSTRACT

The present disclosure relates generally to trans-cyclooctene-modified bispecific antibodies which have applications, e.g., in the treatment of cancer, tumor growth, and immunotherapy.

### CROSS REFERENCE TO RELATED APPLICATIONS

This application claims the benefit under 35 U.S.C. § 119(e) to U.S. Provisional Application No. 63/276,401, filed Nov. 5, 2021, which is incorporated by reference in its entirety.

### FIELD

The present disclosure relates generally to trans-cyclooctene-modified bispecific antibodies which have applications, e.g., in the treatment of cancer, tumor growth, and immunotherapy.

### BACKGROUND

Bioorthogonal conjugation or click reactions are selective and orthogonal (non-interacting with) functionalities found in biological systems, and have found use in various applications in the fields of chemistry, chemical biology, molecular diagnostics, and medicine, where they can be used to facilitate the selective manipulation of molecules, cells, particles and surfaces, and the tagging and tracking of biomolecules in vitro and in vivo. These reactions include the Staudinger ligation, the azide-cyclooctyne cycloaddition, and the inverse-electron-demand Diels-Alder reaction.

### SUMMARY

Provided herein are trans-cyclooctene-modified bispecific antibodies which comprise a bispecific antibody portion having a first antigen target binding moiety and a second antigen target binding moiety, and the antibody portion further comprises at least one trans-cyclooctene moiety covalently bonded thereto optionally via a linker.

In certain embodiments, provided is a system comprising the trans-cyclooctene-modified bispecific antibody as described herein and a tetrazine activator.

The tetrazine activator may be formulated for systemic administration, or is administered via a therapeutic support composition, where the therapeutic support composition comprises a biocompatible support and a tetrazine-containing group.

In certain embodiments, the first antigen target binding moiety of the trans-cyclooctene-modified bispecific antibodies is capable of targeting a specific antigen at a treatment site or on a cell in a subject. In certain embodiments, the trans-cyclooctene moiety substantially blocks or inhibits the second antigen target binding moiety from effectively binding to its target. Once the trans-cyclooctene-modified bispecific antibody binds to the treatment site, contact with a tetrazine activator can release the trans-cyclooctene moiety, thus exposing the second antigen target binding moiety, allowing it to bind its target.

In some embodiments, provided is a method for treating cancer or enhancing or eliciting an immune response, comprising administering to a subject in need thereof, a trans-cyclooctene-modified bispecific antibody as described herein to the subject, and administering to the subject a tetrazine activator. Administration of the tetrazine activator can be prior to or after administration of the trans-cyclooctene-modified bispecific antibody.

In some embodiments, the cancer is metastatic. In some embodiments the cancer is melanoma, renal cancer, prostate cancer, ovarian cancer, endometrial carcinoma, breast cancer, glioblastoma, lung cancer, soft tissue sarcoma, fibrosarcoma, osteosarcoma, pancreatic cancer, gastric carcinoma, squamous cell carcinoma of head/neck, anal/vulvar carcinoma, esophageal carcinoma, pancreatic adenocarcinoma, cervical carcinoma, hepatocellular carcinoma, Kaposi's sarcoma, Non-Hodgkin's lymphoma, Hodgkin's lymphoma, Wilm's tumor/neuroblastoma, bladder cancer, thyroid adenocarcinoma, pancreatic neuroendocrine tumors, prostatic adenocarcinoma, nasopharyngeal carcinoma, or cutaneous T-cell lymphoma.

In some embodiments, the cancer is a melanoma, renal cancer, prostate cancer, ovarian cancer, breast cancer, glioma, lung cancer, soft tissue carcinoma, soft tissue sarcoma, osteosarcoma, or pancreatic cancer.

In some embodiments, the cancer is a solid tumor. In some embodiments, the cancer is a lymphoma or leukemia. In some embodiments, the cancer is a hematological malignancy.

### BRIEF DESCRIPTION OF THE FIGURES

FIG. 1 shows a schematic of an exemplary TCO-Bispecific T cell Engager (BiTE) conjugate.

### DETAILED DESCRIPTION

The following description sets forth exemplary embodiments of the present technology. It should be recognized, however, that such description is not intended as a limitation on the scope of the present disclosure but is instead provided as a description of exemplary embodiments.

### 1. Definitions

It is appreciated that certain features, which are, for clarity, described in the context of separate embodiments, may also be provided in combination in a single embodiment. Conversely, various features, which are, for brevity, described in the context of a single embodiment, may also be provided separately or in any suitable sub-combination. All combinations of the embodiments pertaining to the invention are specifically embraced and are disclosed herein just as if each and every combination was individually and explicitly disclosed, to the extent that such combinations embrace subject matter that are, for example, compounds that are stable compounds (i.e., compounds that can be made, isolated, characterized, and tested for biological activity). In addition, all sub-combinations of the various embodiments and elements thereof (e.g., elements of the chemical groups listed in the embodiments describing such variables) are also specifically embraced and are disclosed herein just as if each and every such sub-combination was individually and explicitly disclosed herein.

### A. Definitions

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “an” and “the” include plural references unless the context clearly dictates otherwise. The present disclosure also contemplates other embodiments “comprising,” “consisting of” and “consisting essentially of,” the embodiments or elements presented herein, whether explicitly set forth or not.

The modifier “about” used in connection with a quantity is inclusive of the stated value and has the meaning dictated by the context (for example, it includes at least the degree of error associated with the measurement of the particular quantity). The modifier “about” should also be considered as disclosing the range defined by the absolute values of the two endpoints. For example, the expression “from about 2 to about 4” also discloses the range “from 2 to 4.” The term “about” may refer to plus or minus 10% of the indicated number. For example, “about 10%” may indicate a range of 9% to 11%, and “about 1” may mean from 0.9-1.1. Other meanings of “about” may be apparent from the context, such as rounding off, so, for example “about 1” may also mean from 0.5 to 1.4.

The conjunctive term “or” includes any and all combinations of one or more listed elements associated by the conjunctive term. For example, the phrase “an apparatus comprising A or B” may refer to an apparatus including A where B is not present, an apparatus including B where A is not present, or an apparatus where both A and B are present. The phrases “at least one of A, B, . . . and N” or “at least one of A, B, . . . N, or combinations thereof” are defined in the broadest sense to mean one or more elements selected from the group comprising A, B, . . . and N, that is to say, any combination of one or more of the elements A, B, . . . or N including any one element alone or in combination with one or more of the other elements which may also include, in combination, additional elements not listed.

Definitions of specific functional groups and chemical terms are described in more detail below. For purposes of this disclosure, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March March's Advanced Organic Chemistry, 5^{th} Edition, John Wiley & Sons, Inc., New York, 2001; Larock, Comprehensive Organic Transformations, VCH Publishers, Inc., New York, 1989; Carruthers, Some Modern Methods of Organic Synthesis, 3^{rd} Edition, Cambridge University Press, Cambridge, 1987; the entire contents of each of which are incorporated herein by reference.

The term “alkoxy” as used herein, refers to an alkyl group, as defined herein, appended to the parent molecular moiety through an oxygen atom. Representative examples of alkoxy include, but are not limited to, methoxy, ethoxy, propoxy, 2-propoxy, butoxy, and tert-butoxy.

The term “alkyl” as used herein, means a straight or branched, saturated hydrocarbon chain containing from 1 to 30 carbon atoms. The term “lower alkyl” or “C₁-C₆-alkyl” means a straight or branched chain hydrocarbon containing from 1 to 6 carbon atoms. The term “C₁-C₃-alkyl” means a straight or branched chain hydrocarbon containing from 1 to 3 carbon atoms. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, and n-decyl.

The term “alkenyl” as used herein, means a hydrocarbon chain containing from 2 to 30 carbon atoms with at least one carbon-carbon double bond. The alkenyl group may be substituted or unsubstituted. For example, the alkenyl group may be substituted with an aryl group, such as a phenyl.

The term “alkynyl,” as used herein, refers to straight or branched monovalent hydrocarbyl groups having from 2 to 30 carbon atoms, such as 2 to 20, or 2 to 10 carbon atoms and having at least 1 site of triple bond unsaturation. The term “alkyne” also includes non-aromatic cycloalkyl groups of from 5 to 20 carbon atoms, such as from 5 to 10 carbon atoms, having single or multiple rings and having at least one triple bond.

Examples of such alkynyl groups include, but are not limited to acetylenyl (—C≡CH), and propargyl (—CH₂C≡CH), and cycloalkynyl moieties, such as, but not limited to, substituted or unsubstituted cyclooctyne moieties.

The term “alkoxyalkyl” as used herein, refers to an alkoxy group, as defined herein, appended to the parent molecular moiety through an alkyl group, as defined herein.

The term “alkylene” as used herein, refers to a divalent group derived from a straight or branched chain hydrocarbon of 1 to 30 carbon atoms, for example, of 2 to 10 carbon atoms. Representative examples of alkylene include, but are not limited to, —CH₂—, —CH(CH₃)—, —C(CH₃)₂—, —CH₂CH₂—, —CH(CH₃)CH₂—, —C(CH₃)₂CH₂—, —CH₂CH₂CH₂—, —CH(CH₃)CH₂CH₂—, —C(CH₃)₂CH₂CH₂—, —CH₂C(CH₃)₂CH₂—, —CH₂CH₂CH₂CH₂—, and —CH₂CH₂CH₂CH₂CH₂—.

The term “amino acid” refers to both natural and unnatural amino acids, protected natural and unnatural amino acids, as well as amino acid analogs and amino acid mimetics that function in a manner similar to the naturally occurring amino acids. Naturally encoded amino acids include 20 common amino acids (alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine and valine) and pyrrolidine and selenocysteine. Amino acid analogs refer to compounds having the same basic chemical structure as a naturally occurring amino acid, i.e., by way of example only, an α-carbon attached to a hydrogen, carboxyl group, amino group, and R group. Such analogs can have a modified R group (e.g., norleucine as an example) or retain a modified peptide backbone while retaining the same basic chemical structure as a natural amino acid. Non-limiting examples of amino acid analogs include citrulline, homoserine, norleucine, methionine sulfoxide, methionine methylsulfonium, homophenylalanine, ornithine, formyl glycine, phenyl glycine, para-azidophenyl glycine, para-azidophenylalanine, para-acetophenylalanine, 4-(3-methyl-(1,2,4,5-tetrazine))-phenylglyine, and 4-(3-methyl-(1,2,4,5-tetrazine))-phenylalanine.

The term “aryl” as used herein, refers to a phenyl group, or bicyclic aryl or tricyclic aryl fused ring systems. Bicyclic fused ring systems are exemplified by a phenyl group appended to the parent molecular moiety and fused to a phenyl group. Tricyclic fused ring systems are exemplified by a phenyl group appended to the parent molecular moiety and fused to two other phenyl groups. Representative examples of bicyclic aryls include, but are not limited to, naphthyl. Representative examples of tricyclic aryls include, but are not limited to, anthracenyl. The monocyclic, bicyclic, and tricyclic aryls are connected to the parent molecular moiety through any carbon atom contained within the rings, and can be unsubstituted or substituted.

The term “azide” as used herein, refers to the functional group —N₃.

The term “cycloalkyl” as used herein, refers to a carbocyclic ring system containing three to ten carbon atoms, zero heteroatoms and zero double bonds. Representative examples of cycloalkyl include, but are not limited to, cyclopropyl, cyclobutyl, cyclopentyl, cyclohexyl, cycloheptyl, cyclooctyl, cyclononyl and cyclodecyl. “Cycloalkyl” also includes carbocyclic ring systems in which a cycloalkyl group is appended to the parent molecular moiety and is fused to an aryl group as defined herein, a heteroaryl group as defined herein, or a heterocycle as defined herein.

The term “cycloalkenyl” as used herein, means a non-aromatic monocyclic or multicyclic ring system containing at least one carbon-carbon double bond and preferably having from 5-10 carbon atoms per ring. Exemplary monocyclic cycloalkenyl rings include cyclopentenyl, cyclohexenyl or cycloheptenyl.

The term “cyclooctene” as used herein, refers to a substituted or unsubstituted non-aromatic cyclic alkyl group of 8 carbon atoms, having a single ring with a double bond. Examples of such cyclooctene groups include, but are not limited to, substituted or unsubstituted trans-cyclooctene (TCO).

The term “fluoroalkyl” as used herein, means an alkyl group, as defined herein, in which one, two, three, four, five, six, seven or eight hydrogen atoms are replaced by fluorine. Representative examples of fluoroalkyl include, but are not limited to, 2-fluoroethyl, 2,2,2-trifluoroethyl, trifluoromethyl, difluoromethyl, pentafluoroethyl, and trifluoropropyl such as 3,3,3-trifluoropropyl.

The term “alkoxyfluoroalkyl” as used herein, refers to an alkoxy group, as defined herein, appended to the parent molecular moiety through a fluoroalkyl group, as defined herein.

The term “fluoroalkoxy” as used herein, means at least one fluoroalkyl group, as defined herein, is appended to the parent molecular moiety through an oxygen atom. Representative examples of fluoroalkyloxy include, but are not limited to, difluoromethoxy, trifluoromethoxy and 2,2,2-trifluoroethoxy.

The term “halogen” or “halo” as used herein, means Cl, Br, I, or F.

The term “haloalkyl” as used herein, means an alkyl group, as defined herein, in which one, two, three, four, five, six, seven or eight hydrogen atoms are replaced by a halogen.

The term “haloalkoxy” as used herein, means at least one haloalkyl group, as defined herein, is appended to the parent molecular moiety through an oxygen atom.

The term “heteroalkyl” as used herein, means an alkyl group, as defined herein, in which one or more of the carbon atoms has been replaced by a heteroatom selected from S, Si, O, P and N. The heteroatom may be oxidized. Representative examples of heteroalkyls include, but are not limited to, alkyl ethers, secondary and tertiary alkyl amines, and alkyl sulfides.

The term “heteroaryl” as used herein, refers to an aromatic monocyclic ring or an aromatic bicyclic ring system or an aromatic tricyclic ring system. The aromatic monocyclic rings are five or six membered rings containing at least one heteroatom independently selected from the group consisting of N, O and S (e.g. 1, 2, 3, or 4 heteroatoms independently selected from O, S, and N). The five membered aromatic monocyclic rings have two double bonds and the six membered aromatic monocyclic rings have three double bonds. The bicyclic heteroaryl groups are exemplified by a monocyclic heteroaryl ring appended to the parent molecular moiety and fused to a monocyclic cycloalkyl group, as defined herein, a monocyclic aryl group, as defined herein, a monocyclic heteroaryl group, as defined herein, or a monocyclic heterocycle, as defined herein. The tricyclic heteroaryl groups are exemplified by a monocyclic heteroaryl ring appended to the parent molecular moiety and fused to two of a monocyclic cycloalkyl group, as defined herein, a monocyclic aryl group, as defined herein, a monocyclic heteroaryl group, as defined herein, or a monocyclic heterocycle, as defined herein. Representative examples of monocyclic heteroaryl include, but are not limited to, pyridinyl (including pyridin-2-yl, pyridin-3-yl, pyridin-4-yl), pyrimidinyl, pyrazinyl, thienyl, furyl, thiazolyl, thiadiazolyl, isoxazolyl, pyrazolyl, and 2-oxo-1,2-dihydropyridinyl. Representative examples of bicyclic heteroaryl include, but are not limited to, chromenyl, benzothienyl, benzodioxolyl, benzotriazolyl, quinolinyl, thienopyrrolyl, thienothienyl, imidazothiazolyl, benzothiazolyl, benzofuranyl, indolyl, quinolinyl, imidazopyridine, benzooxadiazolyl, and benzopyrazolyl. Representative examples of tricyclic heteroaryl include, but are not limited to, dibenzofuranyl and dibenzothienyl. The monocyclic, bicyclic, and tricyclic heteroaryls are connected to the parent molecular moiety through any carbon atom or any nitrogen atom contained within the rings, and can be unsubstituted or substituted.

The term “heterocycle” or “heterocyclic” as used herein, means a monocyclic heterocycle, a bicyclic heterocycle, or a tricyclic heterocycle. The monocyclic heterocycle is a three-, four-, five-, six-, seven-, or eight-membered ring containing at least one heteroatom independently selected from the group consisting of O, N, and S. The three- or four-membered ring contains zero or one double bond, and one heteroatom selected from the group consisting of O, N, and S. The five-membered ring contains zero or one double bond and one, two or three heteroatoms selected from the group consisting of O, N and S. The six-membered ring contains zero, one or two double bonds and one, two, or three heteroatoms selected from the group consisting of O, N, and S. The seven- and eight-membered rings contains zero, one, two, or three double bonds and one, two, or three heteroatoms selected from the group consisting of O, N, and S. Representative examples of monocyclic heterocycles include, but are not limited to, azetidinyl, azepanyl, aziridinyl, diazepanyl, 1,3-dioxanyl, 1,3-dioxolanyl, 1,3-dithiolanyl, 1,3-dithianyl, 1,3-dimethylpyrimidine-2,4(1H,3H)-dione, imidazolinyl, imidazolidinyl, isothiazolinyl, isothiazolidinyl, isoxazolinyl, isoxazolidinyl, morpholinyl, oxadiazolinyl, oxadiazolidinyl, oxazolinyl, oxazolidinyl, oxetanyl, piperazinyl, piperidinyl, pyranyl, pyrazolinyl, pyrazolidinyl, pyrrolinyl, pyrrolidinyl, tetrahydrofuranyl, tetrahydropyranyl, tetrahydropyridinyl, tetrahydrothienyl, thiadiazolinyl, thiadiazolidinyl, 1,2-thiazinanyl, 1,3-thiazinanyl, thiazolinyl, thiazolidinyl, thiomorpholinyl, 1,1-dioxidothiomorpholinyl (thiomorpholine sulfone), thiopyranyl, and trithianyl. The bicyclic heterocycle is a monocyclic heterocycle fused to a phenyl group, or a monocyclic heterocycle fused to a monocyclic cycloalkyl, or a monocyclic heterocycle fused to a monocyclic cycloalkenyl, or a monocyclic heterocycle fused to a monocyclic heterocycle, or a spiro heterocycle group, or a bridged monocyclic heterocycle ring system in which two non-adjacent atoms of the ring are linked by an alkylene bridge of 1, 2, 3, or 4 carbon atoms, or an alkenylene bridge of two, three, or four carbon atoms.

Representative examples of bicyclic heterocycles include, but are not limited to, benzopyranyl, benzothiopyranyl, chromanyl, 2,3-dihydrobenzofuranyl, 2,3-dihydrobenzothienyl, 2,3-dihydroisoquinoline, 2-azaspiro[3.3]heptan-2-yl, azabicyclo[2.2.1]heptyl (including 2-azabicyclo[2.2.1]hept-2-yl), 2,3-dihydro-1H-indolyl, isoindolinyl, octahydrocyclopenta[c]pyrrolyl, octahydropyrrolopyridinyl, and tetrahydroisoquinolinyl. Tricyclic heterocycles are exemplified by a bicyclic heterocycle fused to a phenyl group, or a bicyclic heterocycle fused to a monocyclic cycloalkyl, or a bicyclic heterocycle fused to a monocyclic cycloalkenyl, or a bicyclic heterocycle fused to a monocyclic heterocycle, or a bicyclic heterocycle in which two non-adjacent atoms of the bicyclic ring are linked by an alkylene bridge of 1, 2, 3, or 4 carbon atoms, or an alkenylene bridge of two, three, or four carbon atoms. Examples of tricyclic heterocycles include, but are not limited to, octahydro-2,5-epoxypentalene, hexahydro-2H-2,5-methanocyclopenta[b]furan, hexahydro-1H-1,4-methanocyclopenta[c]furan, aza-adamantane (1-azatricyclo[3.3.1.1^{3,7}]decane), and oxa-adamantane (2-oxatricyclo[3.3.1.1^{3,7}]decane). The monocyclic, bicyclic, and tricyclic heterocycles are connected to the parent molecular moiety through any carbon atom or any nitrogen atom contained within the rings, and can be unsubstituted or substituted.

The term “hydroxyl” as used herein, means an —OH group.

The term “hydroxyalkyl” as used herein, means an alkyl group, as defined herein, in which one, two, three, four, five, six, seven or eight hydrogen atoms are replaced by a hydroxyl group.

In some instances, the number of carbon atoms in a hydrocarbyl substituent (e.g., alkyl or cycloalkyl) is indicated by the prefix “Cₓ-C_{y}—” or “C_{x-y},” wherein x is the minimum and y is the maximum number of carbon atoms in the substituent. Thus, for example, “C₁-C₃-alkyl” and “C₁₋₃alkyl” refer to an alkyl substituent containing from 1 to 3 carbon atoms. The two conventions “Cₓ-C_{y}—” and “Cₓ y” are used interchangeably and have the same meaning.

The term “substituted” refers to a group that may be further substituted with one or more non-hydrogen substituent groups. Substituent groups include, but are not limited to, halogen, ═O, ═S, cyano, nitro, fluoroalkyl, alkoxyfluoroalkyl, fluoroalkoxy, alkyl, alkenyl, alkynyl, haloalkyl, haloalkoxy, heteroalkyl, cycloalkyl, cycloalkenyl, aryl, heteroaryl, heterocycle, cycloalkylalkyl, heteroarylalkyl, arylalkyl, hydroxy, hydroxyalkyl, alkoxy, alkoxyalkyl, alkylene, aryloxy, phenoxy, benzyloxy, amino, alkylamino, acylamino, aminoalkyl, arylamino, sulfonylamino, sulfinylamino, sulfonyl, alkylsulfonyl, arylsulfonyl, aminosulfonyl, sulfinyl, —COOH, ketone, amide, carbamate, and acyl.

The term “tetrazine” refers to a substituted or unsubstituted aromatic cyclic group of 2 carbon atoms and 4 nitrogen atoms, having a single ring with three double bonds. Examples of tetrazine groups include 1,2,3,4-tetrazine and 1,2,4,5-tetrazine. As used herein, 1,2,4,5-tetrazine is referred to as a “Tz” group.

The term “selectively delivering” refers to delivering an agent (e.g., a trans-cyclooctene-modified bispecific antibody) to an organ or tissue (or portion thereof) in need of treatment or diagnosis, without significant binding to other non-target organs or tissues (or portions thereof).

The term “diagnostic agent” refers to agents that assist in diagnosing conditions or diseases. Representative diagnostic agents include imaging agents such as paramagnetic agents, optical probes, radionuclides, and the like. Paramagnetic agents are imaging agents that are magnetic under an externally applied field. Examples of paramagnetic agents include, but are not limited to, iron particles including iron nanoparticles and iron microparticles. Optical probes are fluorescent compounds that can be detected by excitation at one wavelength of radiation and detection at a second, different, wavelength of radiation. Optical probes of the present disclosure include, but are not limited to, Cy5.5, Alexa 680, Cy5, DiD (1,1′-dioctadecyl-3,3,3′,3′-tetramethylindodicarbocyanine perchlorate) and DiR (1,1′-dioctadecyl-3,3,3′,3′-tetramethylindotricarbocyanine iodide). Other optical probes include quantum dots. Radionuclides are elements that undergo detectable radioactive decay. Radionuclides useful in embodiments of the present disclosure include, but are not limited to, ³H, ¹¹C, ¹³N, ¹⁸F, ¹⁹F, ⁶⁰Co, ⁶⁴Cu, ⁶⁷Cu, ⁶⁸Ga, ⁸²Rb, ⁸⁹Zr, ⁹⁰Sr, ⁹⁰Y, ⁹⁹TC, ^{99m}Td, ¹¹¹In, ¹²³I, ¹²⁴I, ¹²⁵I, ¹²⁹I, ¹³¹I, ¹³⁷Cs ¹⁷⁷Lu, ¹⁸⁶Re, ¹⁸⁸Re, ²¹¹At, Rn, Ra, Th, U, Pu and ²⁴¹Am.

The term “targeting agent” refers to a chemical or biological agent that specifically binds to a target (e.g., a targeted organ or tissue), thereby forming a stable association between the targeting agent and the specific target. By “stably associated” or “stable association” is meant that a moiety is bound to or otherwise associated with another moiety or structure under standard physiological conditions. Bonds may include covalent bonds and non-covalent interactions, such as, but not limited to, ionic bonds, hydrophobic interactions, hydrogen bonds, van der Waals forces (e.g., London dispersion forces), dipole-dipole interactions, and the like. A targeting agent may be a member of a specific binding pair, such as, but are not limited to: a member of a receptor/ligand pair; a ligand-binding portion of a receptor; a member of an antibody/antigen pair; an antigen-binding fragment of an antibody; a hapten; a member of a lectin/carbohydrate pair; a member of an enzyme/substrate pair; biotin/avidin; biotin/streptavidin; digoxin/antidigoxin; a member of a DNA or RNA aptamer binding pair; a member of a peptide aptamer binding pair; and the like. Targeting agents include ligands that specifically bind (or substantially specifically bind) a particular clinically-relevant target receptor or cell surface target. The ligand can be an antibody, peptide, nucleic acid, phage, bacteria, virus, or other molecule with a specific affinity for a target receptor or cell surface target. Examples of receptors and cell surface targets include, but are not limited to, PD-1, CTLA-4, HER2/neu, HER1/EGFR, VEGFR, 4-1BB, GITR, LT4-human mAb directed against the inhibitory immune checkpoint receptor immunoglobulin-like transcript 4 (ILT4; leukocyte immunoglobulin-like receptor subfamily B member 2, LILRB2, lymphocyte immunoglobulin-like receptor 2, LIR2, monocyte/macrophage immunoglobulin-like receptor 10, MIR-10, CD85d, or other cellular receptors or cell surface targets. Additional examples are included in various embodiments disclosed herein.

The term “targeted organ or tissue” refers to an organ or tissue that is being targeted for delivery of the ns-cyclooctene-modified bispecific antibody. Representative organs and tissues for targeting include those that can be targeted by chemical or biological targeting agents, as well as those organs and tissues that cannot be targeted by chemical or biological targeting agents.

The term “implanting” refers to surgical implantation into a subject's body.

The term “contacting” or “contact” refers to the process of bringing into contact at least two distinct species such that they can interact with each other, such as in a non-covalent or covalent binding interaction or binding reaction. It should be appreciated, however, the resulting complex or reaction product can be produced directly from an interaction or a reaction between the added reagents or from an intermediate from one or more of the added reagents or moieties, which can be produced in the contacting mixture.

The term “binding agent” refers to an agent having a functional group capable of forming a covalent bond to a complementary functional group of another binding agent in a biological environment. Binding between binding agents in a biological environment may also be referred to as bioconjugation. Binding agents include bioorthogonal binding agents, which are binding agents having bioorthogonal functional groups. Bioorthogonal functional groups of bioorthogonal binding agents selectively react with a complementary bioorthogonal functional group of another bioorthogonal binding partner. Selective reaction between bioorthogonal binding partners can minimize side reactions with other binding agents, biological compounds, or other non-complementary bioorthogonal binding agents or non-complementary bioorthogonal functional groups. Bioorthogonal moieties or functional groups of bioorthogonal binding agents include, but are not limited to, an azide and alkyne for formation of a triazole via Click-chemistry reactions, trans-cyclooctene (TCO) and tetrazine (Tz) (e.g., 1,2,4,5-tetrazine), and others. The binding agents useful in the present disclosure may have a high reactivity with the corresponding binding agent so that the reaction is rapid.

The term “functionalized” refers to a moiety having a functional group attached to the moiety, such as for example a moiety having a binding agent functional group (e.g., a bioorthogonal functional group) attached thereto.

The term “administering” refers to any suitable route of administration to a subject, such as, but not limited to, oral administration, administration as a suppository, topical contact, parenteral, intravenous, intraperitoneal, intramuscular, intralesional, intranasal or subcutaneous administration, intrathecal administration, or the implantation of a slow-release device e.g., a mini-osmotic pump, to the subject.

The term “parenterally,” as used herein, refers to modes of administration which include intravenous, intramuscular, intraperitoneal, intrasternal, subcutaneous and intraarticular injection and infusion.

The term “leaving group” refers to an atom (or a group of atoms) with electron withdrawing ability that can be displaced as a stable species, taking with it the bonding electrons. Examples of suitable leaving groups include halides (e.g., Br, Cl, I), sulfonate esters (e.g., triflate, mesylate, tosylate, and brosylate), and nitrophenols.

The terms “pharmaceutically effective amount” and “therapeutically effective amount” refer to an amount of a compound sufficient to treat a specified disorder or disease or one or more of its symptoms and/or to prevent or reduce the risk of the occurrence or reoccurrence of the disease or disorder or symptom(s) thereof. In reference to tumorigenic proliferative disorders, a pharmaceutically or therapeutically effective amount comprises an amount sufficient to, among other things, cause the tumor to shrink or decrease the growth rate of the tumor.

As used herein, the term “subject,” “patient,” or “organism” includes humans and mammals (e.g., mice, rats, pigs, cats, dogs, and horses). Typical subjects to which an agent(s) of the present disclosure may be administered may include mammals, particularly primates, especially humans. For veterinary applications, suitable subjects may include, for example, livestock such as cattle, sheep, goats, cows, swine, and the like; poultry such as chickens, ducks, geese, turkeys, and the like; and domesticated animals particularly pets such as dogs and cats. For diagnostic or research applications, suitable subjects may include mammals, such as rodents (e.g., mice, rats, hamsters), rabbits, primates, and swine such as inbred pigs and the like.

The term “treating” or “treatment” as used herein means the treating or treatment of a disease or medical condition or symptom(s) thereof in a patient, such as a mammal (particularly a human) that includes: (a) ameliorating the disease or medical condition or symptom(s) thereof, such as, eliminating or causing regression of the disease or medical condition or symptom(s) thereof in a patient; (b) suppressing the disease or medical condition or symptom(s) thereof, for example by, slowing or arresting the development of the disease or medical condition or symptom(s) thereof in a patient; or (c) alleviating a symptom of the disease or medical condition or symptom(s) thereof in a patient.

The term “physiological conditions” is meant to encompass those conditions compatible with living cells, e.g., predominantly aqueous conditions of a temperature, pH, salinity, etc. that are compatible with living cells.

For compounds described herein, groups and substituents thereof may be selected in accordance with permitted valence of the atoms and the substituents, such that the selections and substitutions result in a stable compound, e.g., which does not spontaneously undergo transformation such as by rearrangement, cyclization, elimination, etc.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

The compounds may exist as stereoisomers wherein asymmetric or chiral centers are present. The stereoisomers are “R” or “S” depending on the configuration of substituents around the chiral carbon atom. The terms “R” and “S” used herein are configurations as defined in IUPAC 1974 Recommendations for Section E, Fundamental Stereochemistry, in Pure Appl. Chem., 1976, 45: 13-30. The disclosure contemplates various stereoisomers and mixtures thereof, and these are specifically included within the scope of this invention. Stereoisomers include enantiomers and diastereomers and mixtures of enantiomers or diastereomers. Individual stereoisomers of the compounds may be prepared synthetically from commercially available starting materials, which contain asymmetric or chiral centers or by preparation of racemic mixtures followed by methods of resolution well-known to those of ordinary skill in the art. These methods of resolution are exemplified by (1) attachment of a mixture of enantiomers to a chiral auxiliary, separation of the resulting mixture of diastereomers by recrystallization or chromatography, and optional liberation of the optically pure product from the auxiliary as described in Furniss, Hannaford, Smith, and Tatchell, “Vogel's Textbook of Practical Organic Chemistry,” 5^{th} edition (1989), Longman Scientific & Technical, Essex CM20 2JE, England, or (2) direct separation of the mixture of optical enantiomers on chiral chromatographic columns, or (3) fractional recrystallization methods.

It should be understood that the compounds may possess tautomeric forms as well as geometric isomers, and that these also constitute an aspect of the invention.

The present disclosure also includes isotopically-labeled compounds, which are identical to those recited herein, but for the fact that one or more atoms are replaced by an atom having an atomic mass or mass number different from the atomic mass or mass number usually found in nature. Examples of isotopes suitable for inclusion in the compounds of the invention are hydrogen, carbon, nitrogen, oxygen, phosphorus, sulfur, fluorine, and chlorine, such as, but not limited to, ²H, ³H, ¹³C, ¹⁴C, ¹⁵N, ¹⁸O, ¹⁷O, ³¹P, ³²P, 35S, ¹⁸F, and ³⁶Cl, respectively. Substitution with heavier isotopes such as deuterium, i.e., ²H, can afford certain therapeutic advantages resulting from greater metabolic stability, for example increased in vivo half-life or reduced dosage requirements, and, hence, may be preferred in some circumstances. The compound may incorporate positron-emitting isotopes for medical imaging and positron-emitting tomography (PET) studies for determining the distribution of receptors. Suitable positron-emitting isotopes that can be incorporated are ¹¹C, ¹³N, ¹⁵O, and ¹⁸F. Isotopically-labeled compounds disclosed herein can generally be prepared by conventional techniques known to those skilled in the art or by processes analogous to those described in the accompanying Examples using appropriate isotopically-labeled reagent in place of non-isotopically-labeled reagent.

In certain embodiments, embodied in this disclosure is a system comprising a trans-cyclooctene-modified bispecific antibody and a tetrazine activator for targeted immunotherapy. The bispecific antibody binds to antigens on immune cells, such as T cells, and/or cancer cells to localize the bispecific antibody to a tumor site and stimulate and enhance anti-tumor immunity. Under certain circumstances, undesirable interactions of the bispecific antibody with certain antigens expressed on normal cells can potentially cause adverse systemic and localized toxic effects in treated subjects. However, with a system as provided herein, a trans-cyclooctene-moiety can be covalently bonded to a bispecific antibody at a site which attenuates or inhibits binding of the bispecific antibody to an antigen, thus mitigating potential toxicity in tissues other than the intended treatment site. Localized tetrazine activation at the tumor site releases covalent linkage of the trans-cyclooctene moiety, allowing the bispecific antibody to bind both cognate antigens to elicit immunological anti-tumor response.

### B. Trans-Cyclooctene-Modified Bispecific Antibodies

Provided herein are trans-cyclooctene-modified bispecific antibodies for use in bioorthogonal reactions. In some embodiments, provided is a trans-cyclooctene-modified bispecific antibody comprising a bispecific antibody portion having a first antigen target binding moiety and a second antigen target binding moiety, where the antibody portion further comprises at least one trans-cyclooctene moiety covalently bonded thereto optionally via a linker. In some embodiments, at least one trans-cyclooctene moiety covalently bonded to the bispecific antibody, optionally via a linker, acts to attenuate or inhibit binding to an antigen.

In some embodiments, at least one trans-cyclooctene moiety is covalently bonded to the first antigen target binding moiety optionally via a linker. In some embodiments, at least one trans-cyclooctene moiety is covalently bonded to an antigen binding site on the first antigen target binding moiety optionally via a linker, such as, e.g., in some embodiments, to attenuate or inhibit binding to an antigen. In some embodiments, at least one trans-cyclooctene moiety is covalently bonded to the second antigen target binding moiety optionally via a linker. In some embodiments, at least one trans-cyclooctene moiety is covalently bonded to an antigen binding site on the second antigen target binding moiety optionally via a linker, such as, e.g., in some embodiments, to attenuate or inhibit binding to an antigen.

In some embodiments, the trans-cyclooctene-modified bispecific antibody comprises from 1 to 10, or from 1 to 9, or from 1 to 8, or from 1 to 7, or from 1 to 6, or from 1 to 5, or from 1 to 4, or from 1 to 3, or from 1 to 2 trans-cyclooctene moieties covalently bonded thereto optionally via a linker. The trans-cyclooctene moieties can be the same or different. In some embodiments, the trans-cyclooctene moieties are the same. In some embodiments, the trans-cyclooctene-modified bispecific antibody comprises one trans-cyclooctene moiety covalently bonded thereto optionally via a linker.

In some embodiments, the bispecific antibody comprises at least a portion of an antibody or antibody fragment that binds to an antigen on a hematopoietic cell.

In some embodiments, the bispecific antibody comprises at least a portion of an antibody or antibody fragment that binds to an antigen on a myeloid cell.

In some embodiments, the bispecific antibody comprises at least a portion of an antibody or antibody fragment that binds to an antigen on a lymphocyte.

In some embodiments, the bispecific antibody comprises at least a portion of an antibody or antibody fragment that binds to an antigen on a T-cell or T-cell progenitor.

In some embodiments, the bispecific antibody comprises at least a portion of an antibody or antibody fragment that binds to an antigen on a cancerous cell.

In some embodiments, the bispecific antibody binds to an antigen (e.g., tumor antigen) or 10 target shown in Table 1:

TABLE 1

| Target | NCBI Gene ID |
|---|---|
| 5T4 | 7162 |
| ANG2 | 285 |
| B7-H3 | 80381 |
| BAFFR | 115650 |
| BCMA | 608 |
| BTLA | 151888 |
| CA6 | 765 |
| CCR4 | 1233 |
| CD122 | 3560 |
| CD123 | 3563 |
| CD132 | 3561 |
| CD134 | 7293 |
| CD137 (4-1BB) | 3604 |
| CD16 | 2214 |
| CD19 | 12478 |
| CD20 | 931 |
| CD25 | 3559 |
| CD27 | 939 |
| CD28 | 940 |
| CD3 | 916 |
| CD30 | 943 |
| CD33 | 945 |
| CD38 | 952 |
| CD4 | 920 |
| CD40 | 958 |
| CD40L | 959 |
| CD47 | 961 |
| CD52 | 1043 |
| CD56 | 4684 |
| CD64 | 2209 |
| CD79b | 974 |
| CD8 | 925 |
| CEA | 1048 |
| CLDN18.2 | 51208 |
| CTLA4 | 1493 |
| CXCR4 | 7852 |
| DAP12 | 7305 |
| Dectin-1 (CLEC7A) | 64581 |
| Dectin-2 | 93978 |
| DLL3 | 10683 |
| DLL4 | 54567 |
| DNAM-1 | 10666 |
| EGFR | 1956 |
| ENPP3 | 5169 |
| EpCAM | 4072 |
| FOLR | 2348 |
| GCC | 2984 |
| G-CSFR | 1441 |
| GD2 | gangliosides synthesized |
| | by B4GALNT1 (gene |
| | ID 2583) |
| GITR/TNFRSF18 | 8487 |
| GM-CSFR | 1438 |
| gp100 | 6490 |
| gpA33 | 10223 |
| GPC3 | 2719 |
| HER2 | 2064 |
| HER3 | 30289 |
| ICOS | 29851 |
| IL15R | 3601 |
| LAG3 | 3902 |
| LFA-1 | 3689 |
| Mesothelin | 10232 |
| Mincle | 26253 |
| MUC16 | 94025 |
| MUC17 | 140453 |
| NaPi2b | 10568 |
| NCR1 | 9437 |
| NCR2 | 9436 |
| NCR3 | 259197 |
| NKG2D | 22914 |
| NKp30 | 259197 |
| NKp46 | 17086 |
| OX40 | 7293 |
| PD1 | 5133 |
| PD-L1 | 29126 |
| PSMA | 2346 |
| RANKL | 8600 |
| ROR1 | 491 |
| SLAMF7 | 57823 |
| TAG-72 | |
| TF | 701 |
| TGF-beta | 7040 |
| TIGIT | 201633 |
| TIM3 | 84868 |
| VEGF | 7422 |
| VISTA | 64115 |

In some embodiments, the first antigen target binding moiety is an anti-CD3 antibody, anti-ALB antibody, anti-ANG-2 antibody, anti-CD16 antibody, anti-CD16A antibody, anti-CD47 antibody, anti-DLL4 antibody, anti-DLL5 antibody, anti-EGFR antibody, anti-FAP antibody, anti-FIXa antibody, anti-HER2 ec domain II antibody, anti-IL-17 antibody, anti-IL-23 antibody, anti-IL-4 antibody, anti-PD1 antibody, anti-PDL1 antibody, or anti-TNF-α antibody, or antibody fragment thereof.

In some embodiments, the second antigen target binding moiety is an anti-ANG2 antibody, anti-CD19 antibody, anti-CD30 antibody, anti-CD33 antibody, anti-cMET antibody, anti-CTLA4 antibody, anti-DR5 antibody, anti-FX antibody, anti-HER2 antibody, anti-HER2 ec domain IV antibody, anti-HER3 antibody, anti-ICOS antibody, anti-IL-13 antibody, anti-IL-17 antibody, anti-IL-6 antibody, anti-IL6R antibody, anti-LAG3 antibody, anti-LGR5 antibody, anti-PDL1 antibody, anti-TIM3 antibody, anti-TNF antibody, anti-VEGF antibody, or antibody fragment thereof.

In some embodiments, the bispecific antibody portion is AMF13, NXT007, LY3164530, JNJ-61186372, EMB01, MEHD7945A, RG7221, RG7386, ATN-103, TS-152, ALX-0061, AFM13, AK112, IB1315, ABT122, RO7040547, SAR156597, AZ17, BI 836880, vanucizumab, ABT-165, OMP-305B83, TR009, EMB01, MCLA-158, ZW25, GTB-3550, TG-1801, XmAB23104, AK104, MGD019, XmAb20717, MED15752, MGD013, RG7769, LY3434172, FS118, KN046, or LY3415244.

In some embodiments, the first antigen target binding moiety is an anti-CD3 antibody or anti-CD3 antibody fragment.

In some embodiments, the second antigen target binding moiety is an anti-B7H3 antibody, anti-BCMA antibody, anti-CD123 antibody, anti-CD19 antibody, anti-CD20 antibody, anti-CD33 antibody, anti-CD38 antibody, anti-CEA antibody, anti-CLEC12A antibody, anti-DLL3 antibody, anti-EGFRvIII antibody, anti-EpCAM antibody, anti-FcRH5 antibody, anti-FLT3 antibody, anti-GD2 antibody, anti-GPA33 antibody, anti-GPC antibody, anti-GPC3 antibody, anti-GPRC5D antibody, anti-HER2 antibody, anti-HIV1 Env antibody, anti-MUC16 antibody, anti-P-cadherin, anti-PSMA antibody, or anti-SSTR2 antibody, or antibody fragment thereof.

In some embodiments, the bispecific antibody portion is AFM11, ERY947, RG7802, AMG420, AMG330, AMG212, AMG596, AMG552, AMG673, AMG427, AMG701, MGD006, MGD007, AMG424, MGD011, blinatumomab, AMG562, A-319, RG7828, REGN1979, AR6026, GEN3013, FBTA05, plamotamab, AMG330, AMG673, AMV-564, GEM333, GBR1342, AMG424, AMG420, AMG701, JNJ-64007957, EM801, PF-06863135, REGN5458, TNB-383B, APV0436, MGD006, Xmab14045, JNJ-63709178, MCLA-117, RG6160, AMG427, JNJ-64407564, AMG111, RG7802, solitomab, catumaxomab, pasotuxizumab, HPN-424, AMG160, MOR209, BAY2010112, ertumaxomab, GBR1302, H802, RG6294, PF06671008, MGD007, MGD009, AMG757, REGN4018, AMG596, ERY974, tidutamab, huGD2-BsAB, or MGD014.

In some embodiments, the bispecific antibody portion is a CD3×HER2 bispecific antibody, a PD-L1×EGFR bispecific antibody, a CTLA-4×PD-1 bispecific antibody, or a PD-L1×4-1BB bispecific antibody, a IL-17A×IL-17F bispecific antibody, a VEGF×Ang2 bispecific antibody, a Nectin-4×TLR8 bispecific antibody, a PSMA×CD3 bispecific antibody, a BCMA×CD3 bispecific antibody, a FGFR1×KLB bispecific antibody, a DLL3×CD3 bispecific antibody, a CD38×CD3 bispecific antibody, a FcRL5×CD3 bispecific antibody, a GPRC5D×CD3 bispecific antibody, a GPC3×CD3 bispecific antibody, a HER2×HER3 bispecific antibody, a HER3×IGF-1R bispecific antibody, a EGFR×HER3 bispecific antibody, a CD123×CD3 bispecific antibody, a CD19×CD3 bispecific antibody, a EPCAM×CD3 bispecific antibody, a FIXa×FX bispecific antibody, a EGFR×cMET bispecific antibody.

In some embodiments, the bispecific antibody portion is a CD3×HER2 bispecific antibody, a PD-L1×EGFR bispecific antibody, a CTLA-4×PD-1 bispecific antibody, or a PD-L1×4-1BB bispecific antibody.

In some embodiments, the first antigen target binding moiety targets IL-17A, and the second antigen target binding moiety targets IL-17F.

In some embodiments, the first antigen target binding moiety targets VEGF, and the second antigen target binding moiety targets Ang2.

In some embodiments, the first antigen target binding moiety targets Nectin-4, and the second antigen target binding moiety targets TLR8.

In some embodiments, the first antigen target binding moiety targets PSMA, and the second antigen target binding moiety targets CD3.

In some embodiments, the first antigen target binding moiety targets BCMA, and the second antigen target binding moiety targets CD3.

In some embodiments, the first antigen target binding moiety targets FGFR1, and the second antigen target binding moiety targets KLB.

In some embodiments, the first antigen target binding moiety targets DLL3, and the second antigen target binding moiety targets CD3.

In some embodiments, the first antigen target binding moiety targets CD38, and the second antigen target binding moiety targets CD3.

In some embodiments, the first antigen target binding moiety targets FcRL5, and the second antigen target binding moiety targets CD3.

In some embodiments, the first antigen target binding moiety targets GPRC5D, and the second antigen target binding moiety targets CD3.

In some embodiments, the first antigen target binding moiety targets GPC3, and the second antigen target binding moiety targets CD3.

In some embodiments, the first antigen target binding moiety targets HER2, and the second antigen target binding moiety targets HER3.

In some embodiments, the first antigen target binding moiety targets HER3, and the second antigen target binding moiety targets IGF-1R.

In some embodiments, the first antigen target binding moiety targets EGFR, and the second antigen target binding moiety targets HER3.

In some embodiments, the first antigen target binding moiety targets CD123, and the second antigen target binding moiety targets CD3.

In some embodiments, the first antigen target binding moiety targets CD19, and the second antigen target binding moiety targets CD3.

In some embodiments, the first antigen target binding moiety targets EPCAM, and the second antigen target binding moiety targets CD3.

In some embodiments, the first antigen target binding moiety targets FIXa, and the second antigen target binding moiety targets FX.

In some embodiments, the first antigen target binding moiety targets EGFR, and the second antigen target binding moiety targets cMET.

In some embodiments, the bispecific antibody portion is M1095, BI 836880, SBT6290, CCW702, AMG420, CC-93269, PF-06863135, REGN5458, AMG701, TNB383B, BFKB8488A, BI 764532, AMG 757, AMG424, GBR1342, BFCR4350A, JNJ-64407564, ERY974, MCLA-128, MM-111, istiratumab, duligotumab, flotatuzumab, XmAb14045, JNJ-63709178, blinatumomab, durotuxizumab, catumaxomab, emicizumab, JNJ-61186372, or amivantamab.

In some embodiments, the bispecific antibody portion is AMF13, NXT007, LY3164530, JNJ-61186372, EMB01, MEHD7945A, RG7221, RG7386, ATN-103, TS-152, ALX-0061, AFM13, AK112, 1B1315, ABT122, RO7040547, SAR156597, AZ17, BI 836880, vanucizumab, ABT-165, OMP-305B83, TR009, EMB01, MCLA-158, ZW25, GTB-3550, TG-1801, XmAB23104, AK104, MGD019, XmAb20717, MED15752, MGD013, RG7769, LY3434172, FS118, KN046, LY3415244, AFM11, ERY947, RG7802, AMG420, AMG330, AMG212, AMG596, AMG552, AMG673, AMG427, AMG701, MGD006, MGD007, AMG424, MGD011, blinatumomab, AMG562, A-319, RG7828, REGN1979, AR6026, GEN3013, FBTA05, plamotamab, AMG330, AMG673, AMV-564, GEM333, GBR1342, AMG424, AMG420, AMG701, JNJ-64007957, EM801, PF-06863135, REGN5458, TNB-383B, APV0436, MGD006, Xmab14045, JNJ-63709178, MCLA-117, RG6160, AMG427, JNJ-64407564, AMG111, RG7802, solitomab, catumaxomab, pasotuxizumab, HPN-424, AMG160, MOR209, BAY2010112, ertumaxomab, GBR1302, H802, RG6294, PF06671008, MGD007, MGD009, AMG757, REGN4018, AMG596, ERY974, tidutamab, huGD2-BsAB, MGD014, M1095, BI 836880, SBT6290, CCW702, AMG420, CC-93269, PF-06863135, REGN5458, AMG701, TNB383B, BFKB8488A, BI 764532, AMG757, AMG424, GBR1342, BFCR4350A, JNJ-64407564, ERY974, MCLA-128, MM-111, istiratumab, duligotumab, flotatuzumab, XmAb14045, JNJ-63709178, blinatumomab, durotuxizumab, catumaxomab, emicizumab, JNJ-61186372, or amivantamab.

In some embodiments, the bispecific antibody comprises one or more Fv, Fe, Fab, (Fab′)2, single chain Fv (scFv), single heavy chain antibody (VHH), diabody, triabody, tetrabody, bifunctional hybrid antibody, CDR1, CDR2, CDR3, variable region, framework region, constant region, heavy chain, light chain, alternative scaffold non-antibody molecule (DART, DARPin), or a combination thereof.

In some embodiments, the bispecific antibody comprises a scFv. In some embodiments, the first antigen target binding moiety comprises a scFv. In some embodiments, the second antigen target binding moiety comprises a scFv.

In some embodiments, the bispecific antibody comprises a VHH. In some embodiments, the first antigen target binding moiety comprises a VHH. In some embodiments, the second antigen target binding moiety comprises a VHH.

In some embodiments, the bispecific antibody comprises at least a portion of a Fab fragment. In some embodiments, the first antigen target binding moiety comprises at least a portion of a Fab fragment. In some embodiments, the second antigen target binding moiety comprises at least a portion of a Fab fragment.

In some embodiments, the trans-cyclooctene moiety is covalently bonded to the bispecific antibody via a linker.

In some embodiments, the trans-cyclooctene moiety of the instant disclosure can be covalently bonded (i.e., conjugated) to a Cys or Lys residue in the bispecific antibody, optionally via a linker. The Cys or Lys residue may be naturally occurring in the antibody, such as at a location that impacts the biological activity of the bispecific antibody. For example, a Cys residue may be in one of the CDRs, or in a framework region adjacent a CDR. Once the trans-cyclooctene moiety is conjugated to the antibody, such as via a linker, through the side chain of the Cys residue, the trans-cyclooctene moiety (optionally including a linker) can inhibit the antibody activity through mechanisms such as steric hindrance.

In the event there is no naturally occurring Cys or Lys, the bispecific antibody can be engineered to substitute a natural residue with Cys or Lys. Such a substitution, in some embodiments, is conservative substitution. For instance, Cys can replace an existing Met, Thr, or Ser without having a significant impact on the biological activity of the bispecific antibody. Likewise, Lys can replace an existing Arg. In some embodiments, the substitution is at a framework residue adjacent a CDR. It is contemplated that such a substitution (not within CDR), in particular a conservative substitution, would have minimum effect on the biological activity of the bispecific antibody. On the other hand, in some embodiments, given the size of a potential trans-cyclooctene moiety, the steric hindrance from the trans-cyclooctene moiety (optionally including a linker) conjugated at an adjacent residue can still effectively inhibit the binding of the CDR to the corresponding antigen.

In some embodiments, the bispecific antibody is further engineered to remove naturally occurring Cys or Lys at locations where conjugation of the BML is not desired. For instance, a Cys may be substituted with a Met, Thr or Ser, and a Lys may be substituted with an Arg.

In some embodiments, the bispecific antibody comprises an N-terminal amino acid, wherein the linker is covalently bonded to a N-terminal amino acid.

In some embodiments, the trans-cyclooctene moiety is covalently bonded to the bispecific antibody via a cysteine or lysine residue on the bispecific antibody.

In some embodiments, the linker comprises one or more amino acids.

In some embodiments, the linker comprises a polypeptide.

In some embodiments, the linker comprises one or more of a hydrazone, a hydrazide, a disulfide, a N-succinimidyl-4-(2-pyridyldithio)pentanoate (SPP), a N-succinimidyl-4-(2-pyridyldithio)butyrate (SPDB), a 4-(4′-acetylphenoxy)butanoic acid (AcBut), one or more linear or branched, natural or unnatural amino acid, a valine-citrulline (Val-Cit) moiety, or a phenylalanine-lysine (Phe-Lys) moiety.

In some embodiments, the linker comprises 1 to 100 linking atoms, from 1 to 50 linking atoms, or from 5 to 50 linking atoms, or from 10 to 50 linking atoms, or from 1 to 40 linking atoms, or from 1 to 30 linking atoms, or from 1 to 20 linking atoms, or from 1 to 10 linking atoms, or from 1 to 5 linking atoms, or from 5 to 30 linking atoms, or from 10 to 30 linking atoms, or from 5 to 40 linking atoms, or from 5 to 50 linking atoms, or from 10 to 50 linking atoms.

In some embodiments, the linker comprises one or more chain heteroatoms and one or more alkylene, alkenylene, alkynylene, arylene, heteroarylene, cycloalkylene or heterocycloalkylene moieties; wherein each alkylene, alkenylene, alkynylene, arylene, heteroarylene, cycloalkylene or heterocycloalkylene moiety, may be independently optionally substituted with one to five substituents independently selected from oxo, halo, C₁₋₄ alkyl, C₁₋₄ alkoxy, and C₁₋₄ haloalkyl.

In some embodiments, the linker is an alkylene linker optionally comprising one or more —O—, —S—, amine, ester, amide, carbamate, carbonate, thio-succinimide, or ketone functional group.

In some embodiments, the linker is a linker moiety L as defined herein.

In some embodiments, the linker is of the formula:

—Y¹⁰—(CH₂)_{n′}—Y²⁰—(CH₂)_{m″}—Y³⁰—
wherein:
each of Y¹⁰, Y²⁰, and Y³⁰ are independently a bond, —NR¹¹⁰—, —O—, —S(O)₀₋₂—, —NR¹¹⁰C(O)—, —C(O)NR¹¹⁰—, —OC(O)NR¹¹⁰—, —NR¹¹⁰S(O)₂—, —S(O)₂NR¹¹⁰—, —CR¹²⁰═N—NR¹¹⁰—, —NR¹¹⁰—N═CR¹²⁰—, —C(O)—, —OC(O)—, —OC(O)O—, alkylene, alkenylene, alkynylene, arylene, heteroarylene, cycloalkylene, or heterocycloalkylene; wherein each alkylene, alkenylene, alkynylene, arylene, heteroarylene, cycloalkylene, or heterocycloalkylene is independently optionally substituted with one to five substituents independently selected from oxo, halo, C₁₋₄ alkyl, C₁₋₄ alkoxy, and C₁₋₄ haloalkyl;
each R¹¹⁰ is independently hydrogen, C₁₋₄ alkyl, C₁₋₄ haloalkyl, aryl, heteroaryl, cycloalkyl or heterocyclyl;
each R¹²⁰ is independently hydrogen, C₁₋₄ alkyl, C₁₋₄ haloalkyl, aryl, heteroaryl, cycloalkyl or heterocyclyl; and
n′ and m″ are each independently 0, 1, 2, 3, 4, 5, 6, 7, or 8.

In some embodiments, the trans-cyclooctene moiety is of Formula X:

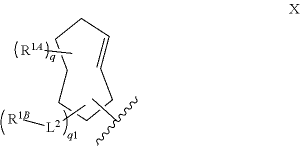

wherein:
R^{1A}, at each occurrence, is independently selected from the group consisting of C₁₋₄alkyl, C₁₋₄haloalkyl, and C₁₋₄alkoxy;
q is 0, 1, or 2;
q1 is 0 or 1;
R^{1B}, at each occurrence, is independently selected from the group consisting of G¹, OH, —NR^{1c}—C₁₋₄alkylene-G¹, —NR^{1c}—C₁₋₄alkylene-N(R^{1d})₂, —NR^{1c}—C₁₋₆alkylene-N(C₁₋₄alkyl)₃⁺, —N(R^{1c})CHR^{1e}CO₂H, —N(R^{1c})—C₁₋₆alkylene-CO₂H, —N(R^{1f})—C₂₋₄alkylene-(N(C₁₋₄alkylene-CO₂H)—C₂₋₄alkylene), —N(C₁₋₄alkylene-CO₂H)₂, —N(R^{1c})CHR^{1e}C(O)OC₁₋₆alkyl, —N(R^{1c})—C₁₋₆alkylene-C(O)OC₁₋₆alkyl, —N(R^{1f})—C₂₋₄alkylene-(N(C₁₋₄alkylene-C(O)OC₁₋₁₆alkyl)-C₂ 4alkylene)ₙ—N(C₁₋₄alkylene-C(O)OC₁₋₆alkyl)₂, —N(R^{1c})—C₁₋₆alkylene-SO₃H, —N(R^{1c})—(CH₂CH₂O)₁₋₃—CH₂CH₂N((CH₂CH₂O)₁₋₃—C₁₋₆alkylene-CO₂H)₂, and —N(R^{1c})—CH(CH₂O—(CH₂CH₂O)₀₋₂—C₁₋₆alkylene-CO₂H)₂;
R^{1c} and R^{1d}, at each occurrence, are independently hydrogen or C₁₋₄alkyl; R^{1e}, at each occurrence, is independently —C₁₋₄alkylene-CO₂H, —C₁₋₄alkylene-CONH₂, or —C₁₋₄alkylene-OH;
R^{1f}, at each occurrence, is independently hydrogen, C₁₋₆alkyl, or C₁₋₄alkylene-CO₂H;
n, at each occurrence, is independently 0, 1, 2, or 3;
L², at each occurrence, is independently selected from the group consisting of —C(O)— and C₁₋₃alkylene; and
G¹, at each occurrence, is independently an optionally substituted heterocyclyl.

In some embodiments, the trans-cyclooctene moiety is of Formula XI:

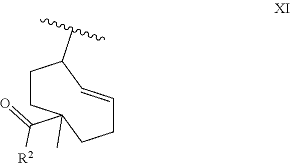

wherein:
R² is —OH, 2-aminoethanesulfonic acid, an N-linked natural or unnatural amino acid, or an optionally substituted ethylenediamine; wherein R² may be optionally further substituted with a polyether.

In some embodiments, the trans-cyclooctene moiety with a portion of the linker comprises a group capable of releasing the trans-cyclooctene moiety from the bispecific antibody portion (such as upon tetrazine activation). In some embodiments, the trans-cyclooctene moiety linked to the bispecific antibody portion via a linker having a —OC(O)— or —OC(O)NH— moiety at the attachments to the trans-cyclooctene moiety.

In some embodiments, the trans-cyclooctene moiety with a linker portion having the Formula XIA or XIB:

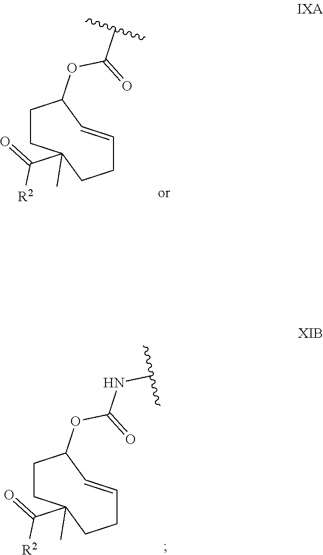

wherein:
R² is —OH, 2-aminoethanesulfonic acid, an N-linked natural or unnatural amino acid, or an optionally substituted ethylenediamine; wherein R² may be optionally further substituted with a polyether. In some embodiments, the remaining portion of the liker comprises an alkylene or polyether with an amino acid terminus for bonding to the bispecific antibody portion.

In some embodiments, the trans-cyclooctene moiety is of Formula:

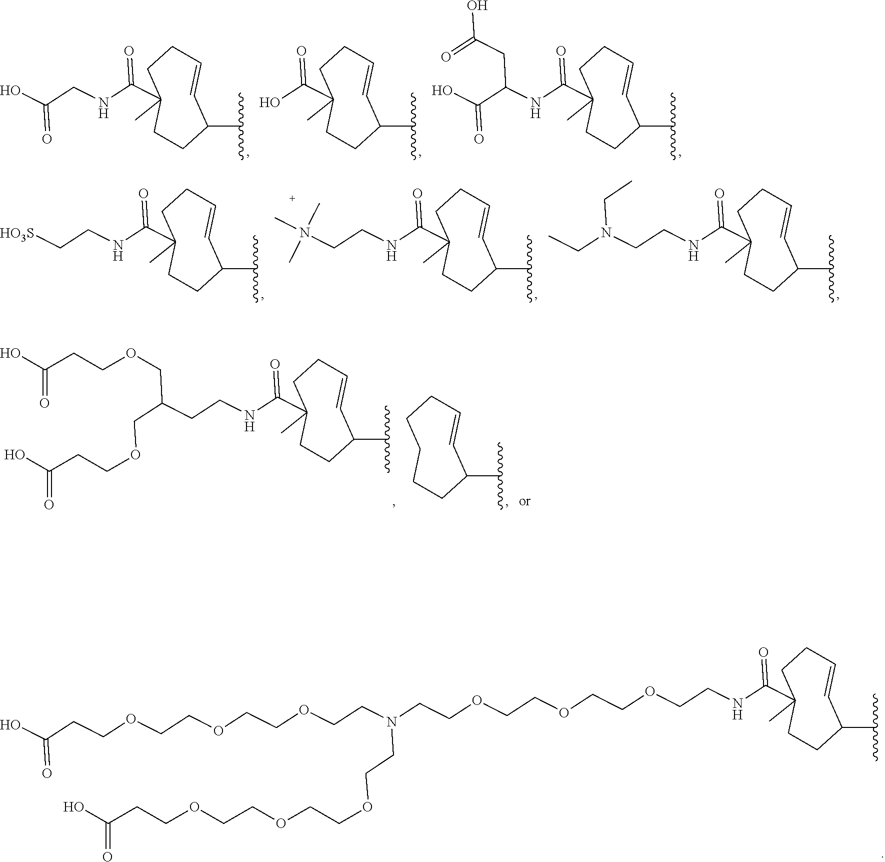

Also provided is a pharmaceutical composition comprising a trans-cyclooctene-modified bispecific antibody as disclosed herein and a pharmaceutically acceptable carrier.

### C. Tetrazine Activators

In certain embodiments, provided is a system comprising the trans-cyclooctene-modified bispecific antibody as described herein and a tetrazine activator. The tetrazine activator may be formulated for systemic administration as a free tetrazine compound or as a tetrazine moiety covalently bonded to an antibody or antibody fragment moiety, optionally via a linker, or is administered via a therapeutic support composition, where the therapeutic support composition comprises a biocompatible support and a tetrazine-containing group.

In certain embodiments, the first antigen target binding moiety of the trans-cyclooctene-modified bispecific antibodies is capable of targeting a specific antigen at a treatment site or on a cell in a subject. In certain embodiments, the trans-cyclooctene moiety substantially blocks or inhibits the second antigen target binding moiety from effectively binding to its target. Once the trans-cyclooctene-modified bispecific antibody binds to the treatment site, contact with a tetrazine activator can release the trans-cyclooctene moiety, thus exposing the second antigen target binding moiety and allowing it to bind its target.

In certain embodiments, the tetrazine activator is formulated for systemic administration.

In certain embodiments, the tetrazine activator is of Formula IV:

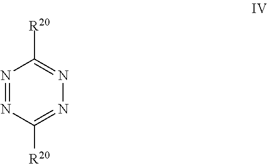

wherein:
each R²⁰ is independently selected from the group consisting of hydrogen, halo, cyano, nitro, alkyl, alkenyl, alkynyl, haloalkyl, heteroalkyl, aryl, heteroaryl, heterocyclyl, cycloalkyl, OR′, SR′, C(═O)R′, C(═S)R′, OC(═O)R′″, SC(═O)R″, OC(═S)R′″, SC(═S)R′″, S(═O)R′, S(═O)₂R′″, S(═O)₂NR′R″, C(═O)O—R′, C(═O)S—R′, C(═S)OR′, C(═S)SR′, C(═O)NR′R″, C(═S)NR′R″, NR′R″, NR′C(═O)R″, NR′C(═S)R″, NR′C(═O)OR″, NR′C(═S)OR″, NR′C(═O)SR″, NR′C(═S)SR″, OC(═O)NR′R″, SC(═O)NR′R″, OC(═S)R′R′″, SC(═S)R′R″, NR′C(═O)NR″R″, and NR′C(═S)NR″R″; wherein each alkyl, alkenyl, alkynyl, haloalkyl, heteroalkyl, aryl, heteroaryl, heterocyclyl, or cycloalkyl is optionally substituted with one to three Z¹;
each Z¹ is independently selected from halo, oxo, cyano, nitro, hydroxy, alkyl, haloalkyl, alkenyl, alkynyl, alkoxy, haloalkoxy, heteroalkyl, aryl, heteroaryl, heterocyclyl, cycloalkyl, OR′, SR′, C(═O)R′, C(═S)R′, OC(═O)R′″, SC(═O)R″, OC(═S)R′″, SC(═S)R′″, S(═O)R′, S(═O)₂R′″, S(═O)₂NR′R″, C(═O)O—R′, C(═O)S—R′, C(═S)O—R′, C(═S)S—R′, C(═O)NR′R″, C(═S)NR′R″, NR′R″, NR′C(═O)R″, NR′C(═S)R″, NR′C(═O)OR″, NR′C(═S)OR″, NR′C(═O)SR″, NR′C(═S)SR″, OC(═O)NR′R″, SC(═O)NR′R″, OC(═S)R′R′″, SC(═S)R′R″, NR′C(═O)NR″R″, and NR′C(═S)NR″R″;
R′ and R″, at each occurrence, are independently selected from hydrogen, aryl, and alkyl; and
R′″, at each occurrence, is independently selected from aryl and alkyl.

In certain embodiments, the each R²⁰ is independently phenyl, pyrimidinyl, triazinyl, oxazolyl, isoxazole, imidazolyl, oxadiazolyl, 6,7-dihydro-5H-pyrrolo[3,4-d]pyrimidinyl, 5,6,7,8-tetrahydropyrido[4,3-d]pyrimidinyl, or 5,6,7,8-tetrahydropyrido[3,4-d]pyrimidinyl; wherein is independently optionally substituted with one to three Z¹.

In certain embodiments, the tetrazine activator is:

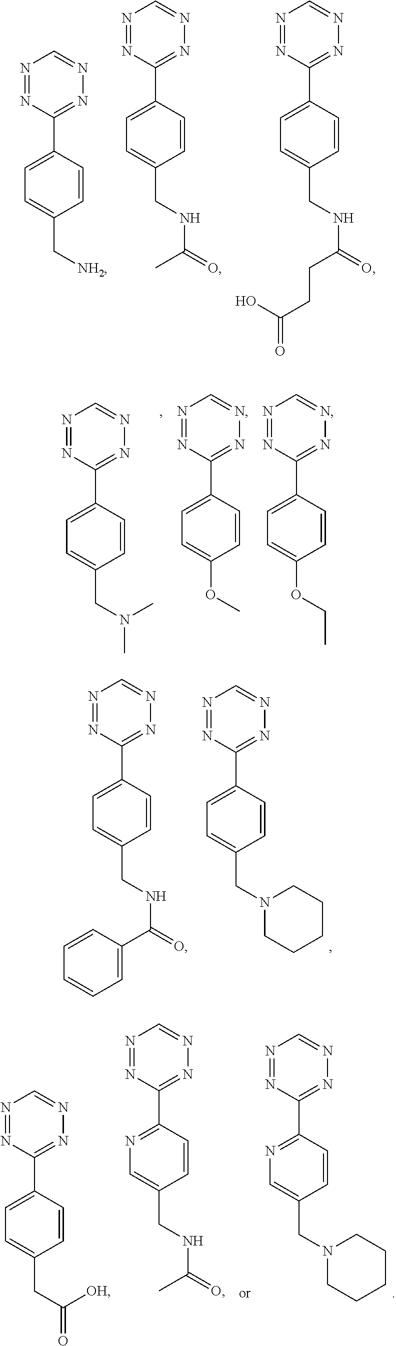

In certain embodiments, the tetrazine activator is:

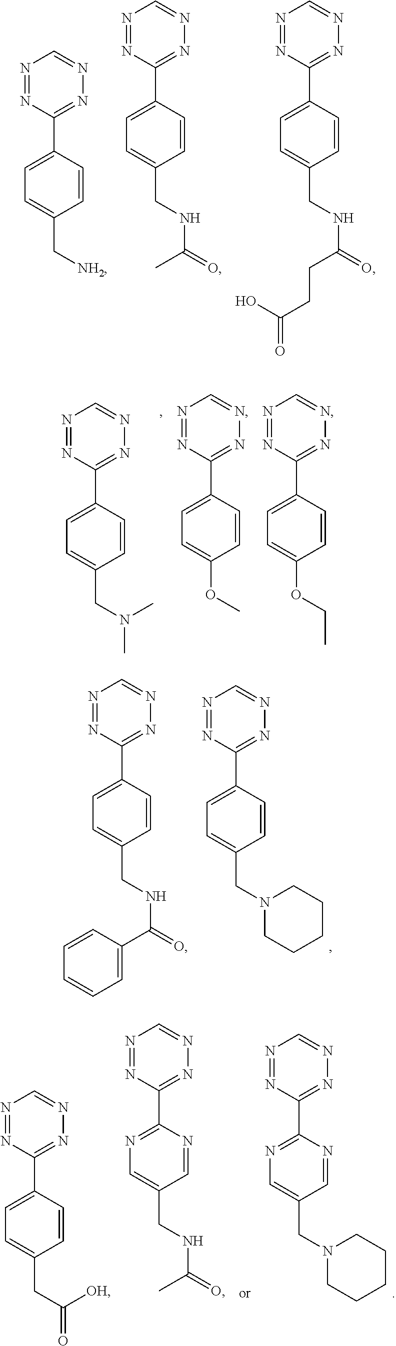

In certain embodiments, the tetrazine activator comprises a biocompatible support, an antibody or antibody fragment moiety, or in certain embodiments an antibody or antibody fragment moiety, covalently bonded to one or more tetrazine moieties.

In certain embodiments, the tetrazine activator is of Formula I, Formula II, or Formula V:

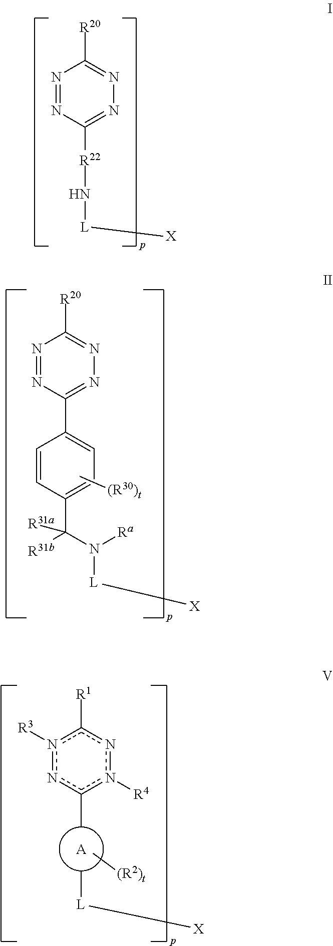

wherein:
ring A is aryl, cycloalkyl, heterocyclyl, or heteroaryl;
the dotted lines represent additional bonds to form a tetrazine when R³ and R⁴ are both absent, or a dihydrotetrazine when R³ and R⁴ are both present; provided that when ring A is aryl, then R³ and R⁴ are both present;
X is a biocompatible support, antibody, or antibody fragment moiety; provided that for Formula I and Formula II, X is not a biocompatible support;
p is 1-150;
L, at each occurrence, is independently a linker;
R¹, at each occurrence, is independently selected from the group consisting of hydrogen, halo, cyano, nitro, alkyl, alkenyl, alkynyl, haloalkyl, heteroalkyl, aryl, heteroaryl, heterocyclyl, cycloalkyl, OR′, SR′, C(═O)R′, C(═S)R′, OC(═O)R′″, SC(═O)R″, OC(═S)R′″, SC(═S)R′″, S(═O)R′, S(═O)₂R′″, S(═O)₂NR′R″, C(═O)O—R′, C(═O)S—R′, C(═S)OR′, C(═S)SR′, C(═O)NR′R″, C(═S)NR′R″, NR′R″, NR′C(═O)R″, NR′C(═S)R″, NR′C(═O)OR″, NR′C(═S)OR″, NR′C(═O)SR″, NR′C(═S)SR″, OC(═O)NR′R″, SC(═O)NR′R″, OC(═S)R′R′″, SC(═S)R′R″, NR′C(═O)NR″R″, and NR′C(═S)NR″R″; wherein each alkyl, alkenyl, alkynyl, haloalkyl, heteroalkyl, aryl, heteroaryl, heterocyclyl, or cycloalkyl is optionally substituted with one to three Z¹;
R², at each occurrence, is independently halo, cyano, nitro, hydroxy, alkyl, haloalkyl, alkenyl, alkynyl, alkoxy, haloalkoxy, heteroalkyl, aryl, heteroaryl, heterocyclyl, cycloalkyl, —C(═O)-alkyl, —C(═O)-haloalkyl, —C(═O)-alkenyl, —C(═O)-alkynyl, —C(═O)-alkoxy, —C(═O)-haloalkoxy, —C(═O)-heteroalkyl, —C(═O)-aryl, —C(═O)-heteroaryl, —C(═O)-heterocyclyl, or —C(═O)-cycloalkyl; wherein each alkyl, haloalkyl, alkenyl, alkynyl, alkoxy, haloalkoxy, heteroalkyl, aryl, heteroaryl, heterocyclyl, or cycloalkyl is optionally substituted with one to three Z¹;
R³ and R⁴ are both absent; or R³ and R⁴ are each independently hydrogen or a group capable of being removed after a triggering event;
R²⁰, at each occurrence, is independently selected from the group consisting of hydrogen, halogen, cyano, nitro, alkyl, alkenyl, alkynyl, heteroalkyl, aryl, heteroaryl, heterocyclyl, cycloalkyl, cycloalkenyl, CF₃, CF₂—R′, NO₂, OR′, SR′, C(═O)R′, C(═S)R′, OC(═O)R′″, SC(═O)R″, OC(═S)R′″, SC(═S)R′″, S(═O)R′, S(═O)₂R′″, S(═O)₂NR′R″, C(═O)O—R′, C(═O)S—R′, C(═S)O—R′, C(═S)S—R′, C(═O)NR′R″, C(═S)NR′R″, NR′R″, NR′C(═O)R″, NR′C(═S)R″, NR′C(═O)OR″, NR′C(═S)OR″, NR′C(═O)SR″, NR′C(═S)SR″, OC(═O)NR′R″, SC(═O)NR′R″, OC(═S) R′R′″, SC(═S)R′R″, NR′C(═O)NR″R″, and NR′C(═S)NR″R″;
R²², at each occurrence, is independently a linker of 1 to 100 linking atoms optionally comprising one or more ethylene-oxy, amine, ester, amide, carbamate, carbonate, or ketone functional group;
R³⁰, at each occurrence, is independently halogen, cyano, nitro, hydroxy, alkyl, haloalkyl, alkenyl, alkynyl, alkoxy, haloalkoxy, heteroalkyl, aryl, heteroaryl, heterocyclyl, cycloalkyl, or cycloalkenyl; R^{a}, R^{31a} and R^{31b} are each independently hydrogen, C₁-C₆-alkyl, or C₁-C₆-haloalkyl;
each Z¹ is independently selected from halo, oxo, cyano, nitro, hydroxy, alkyl, haloalkyl, alkenyl, alkynyl, alkoxy, haloalkoxy, heteroalkyl, aryl, heteroaryl, heterocyclyl, cycloalkyl, OR′, SR′, C(═O)R′, C(═S)R′, OC(═O)R′″, SC(═O)R″, OC(═S)R′″, SC(═S)R′″, S(═O)R′, S(═O)₂R′″, S(═O)₂NR′R″, C(═O)O—R′, C(═O)S—R′, C(═S)O—R′, C(═S)S—R′, C(═O)NR′R″, C(═S)NR′R″, NR′R″, NR′C(═O)R″, NR′C(═S)R″, NR′C(═O)OR″, NR′C(═S)OR″, NR′C(═O)SR″, NR′C(═S)SR″, OC(═O)NR′R″, SC(═O)NR′R″, OC(═S)R′R′″, SC(═S)R′R″, NR′C(═O)NR″R″, and NR′C(═S)NR″R″;
R′ and R″, at each occurrence, are independently selected from hydrogen, aryl, and alkyl;
R′″, at each occurrence, is independently selected from aryl and alkyl; and
t, at each occurrence, is independently is 0, 1, 2, 3, or 4.

In certain embodiments, the tetrazine activator is of Formula I:

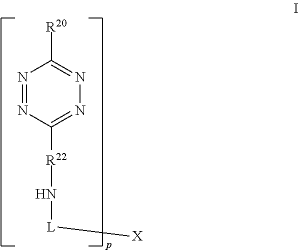

wherein:
X is an antibody or antibody fragment moiety;
p is 1-16;
L, at each occurrence, is independently a linker;
R²⁰, at each occurrence, is independently selected from the group consisting of hydrogen, halogen, cyano, nitro, alkyl, alkenyl, alkynyl, heteroalkyl, aryl, heteroaryl, heterocyclyl, cycloalkyl, cycloalkenyl, CF₃, CF₂—R′, NO₂, OR′, SR′, C(═O)R′, C(═S)R′, OC(═O)R′″, SC(═O)R″, OC(═S)R′″, SC(═S)R′″, S(═O)R′, S(═O)₂R′″, S(═O)₂NR′R″, C(═O)O—R′, C(═O)S—R′, C(═S)O—R′, C(═S)S—R′, C(═O)NR′R″, C(═S)NR′R″, NR′R″, NR′C(═O)R″, NR′C(═S)R″, NR′C(═O)OR″, NR′C(═S)OR″, NR′C(═O)SR″, NR′C(═S)SR″, OC(═O)NR′R″, SC(═O)NR′R″, OC(═S)R′R′″, SC(═S)R′R″, NR′C(═O)NR″R″, and NR′C(═S)NR″R″;
R²², at each occurrence, is independently a linker of 1 to 100 linking atoms optionally comprising one or more ethylene-oxy, amine, ester, amide, carbamate, carbonate, or ketone functional group;
R′ and R″, at each occurrence, are independently selected from hydrogen, aryl, and alkyl; and
R′″ at each occurrence is independently selected from aryl and alkyl.

In one embodiment, the tetrazine activator is of Formula II:

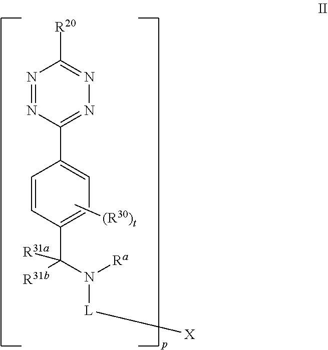

wherein:
X is an antibody or antibody fragment moiety;
p is 1-16;
L, at each occurrence, is independently a linker;
R²⁰, at each occurrence, is independently selected from the group consisting of hydrogen, halogen, cyano, nitro, alkyl, alkenyl, alkynyl, heteroalkyl, aryl, heteroaryl, heterocyclyl, cycloalkyl, cycloalkenyl, CF₃, CF₂—R′, NO₂, OR′, SR′, C(═O)R′, C(═S)R′, OC(═O)R′″, SC(═O)R″, OC(═S)R′″, SC(═S)R′″, S(═O)R′, S(═O)₂R′″, S(═O)₂NR′R″, C(═O)O—R′, C(═O)S—R′, C(═S)O—R′, C(═S)S—R′, C(═O)NR′R″, C(═S)NR′R″, NR′R″, NR′C(═O)R″, NR′C(═S)R″, NR′C(═O)OR″, NR′C(═S)OR″, NR′C(═O)SR″, NR′C(═S)SR″, OC(═O)NR′R″, SC(═O)NR′R″, OC(═S)R′R′″, SC(═S)R′R″, NR′C(═O)NR″R″, and NR′C(═S)NR″R″;
R³⁰, at each occurrence, is independently halogen, cyano, nitro, hydroxy, alkyl, haloalkyl, alkenyl, alkynyl, alkoxy, haloalkoxy, heteroalkyl, aryl, heteroaryl, heterocyclyl, cycloalkyl, or cycloalkenyl;
R^{a}, R^{31a} and R^{31b} are each independently hydrogen, C₁-C₆-alkyl, or C₁-C₆-haloalkyl;
R′ and R″, at each occurrence, are independently selected from hydrogen, aryl, and alkyl;
R′″, at each occurrence, is independently selected from aryl and alkyl; and
t is independently is 0, 1, 2, 3, or 4.

In one embodiment, R²², at each occurrence, is independently a linker of 1 to 100 linking atoms, and can include ethylene-oxy groups, amines, esters, amides, carbamates, carbonates, and ketone functional groups.

In one embodiment, the tetrazine activator is of Formula IIA:

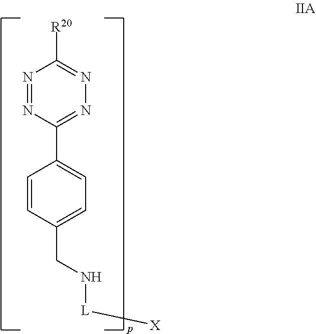

wherein L, p, X, and R²⁰ are each independently as defined herein.

In one embodiment, the tetrazine activator is of Formula IIB:

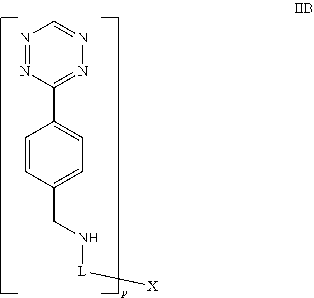

wherein L, p, and X are each independently as defined herein.

In one embodiment, the tetrazine activator is of Formula IIC:

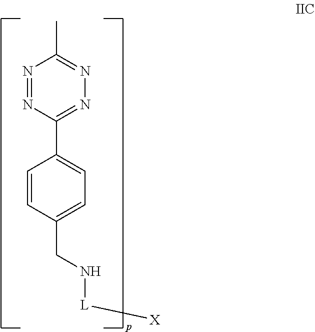

wherein L, p, and X are each independently as defined herein.

In one embodiment, the tetrazine activator is of Formula III:

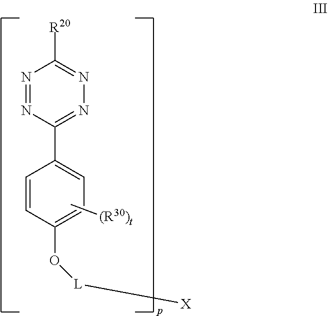

wherein:
X is an antibody or antibody fragment moiety;
p is 1-16;
L, at each occurrence, is independently a linker;
R²⁰, at each occurrence, is independently selected from the group consisting of hydrogen, halogen, cyano, nitro, alkyl, alkenyl, alkynyl, heteroalkyl, aryl, heteroaryl, heterocyclyl, cycloalkyl, cycloalkenyl, CF₃, CF₂—R′, NO₂, OR′, SR′, C(═O)R′, C(═S)R′, OC(═O)R′″, SC(═O)R″, OC(═S)R′″, SC(═S)R′″, S(═O)R′, S(═O)₂R′″, S(═O)₂NR′R″, C(═O)O—R′, C(═O)S—R′, C(═S)O—R′, C(═S)S—R′, C(═O)NR′R″, C(═S)NR′R″, NR′R″, NR′C(═O)R″, NR′C(═S)R″, NR′C(═O)OR″, NR′C(═S)OR″, NR′C(═O)SR″, NR′C(═S)SR″, OC(═O)NR′R″, SC(═O)NR′R″, OC(═S)R′R′″, SC(═S)R′R″, NR′C(═O)NR″R″, and NR′C(═S)NR″R″;
R³⁰, at each occurrence, is independently halogen, cyano, nitro, hydroxy, alkyl, haloalkyl, alkenyl, alkynyl, alkoxy, haloalkoxy, heteroalkyl, aryl, heteroaryl, heterocyclyl, cycloalkyl, or cycloalkenyl;
R′ and R″, at each occurrence, are independently selected from hydrogen, aryl, and alkyl;
R′″, at each occurrence, is independently selected from aryl and alkyl; and
t is independently is 0, 1, 2, 3, or 4.

In some embodiments, the tetrazine activator is represented by Formula IID:

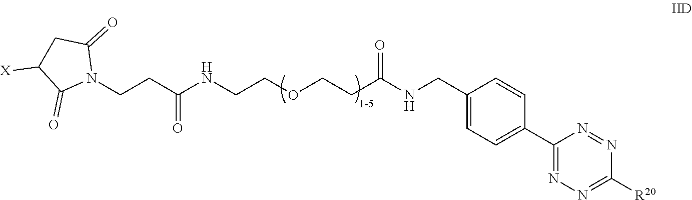

wherein X and R²⁰ are each independently as defined herein. In some embodiments, R²⁰ is methyl.

In some embodiments, X is an antigen-binding protein. In some embodiments, X is an antigen-binding protein which targets HER2.

In some embodiments, the tetrazine activator is of Formula IIE:

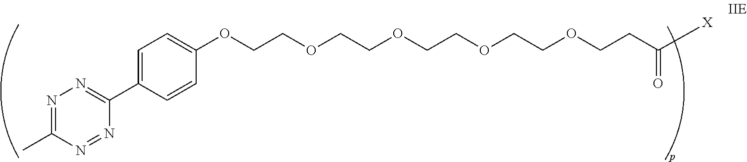

wherein p and X are each independently as defined herein.

In some embodiments, the tetrazine activator is of Formula IIF:

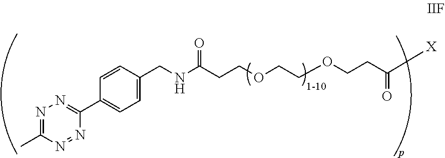

wherein p and X are each independently as defined herein.

In some embodiments, the tetrazine activator is of Formula IIG:

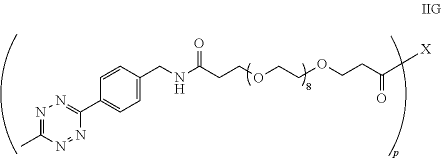

wherein p and X are each independently as defined herein.

In some embodiments of Formula IIA, at least one of:

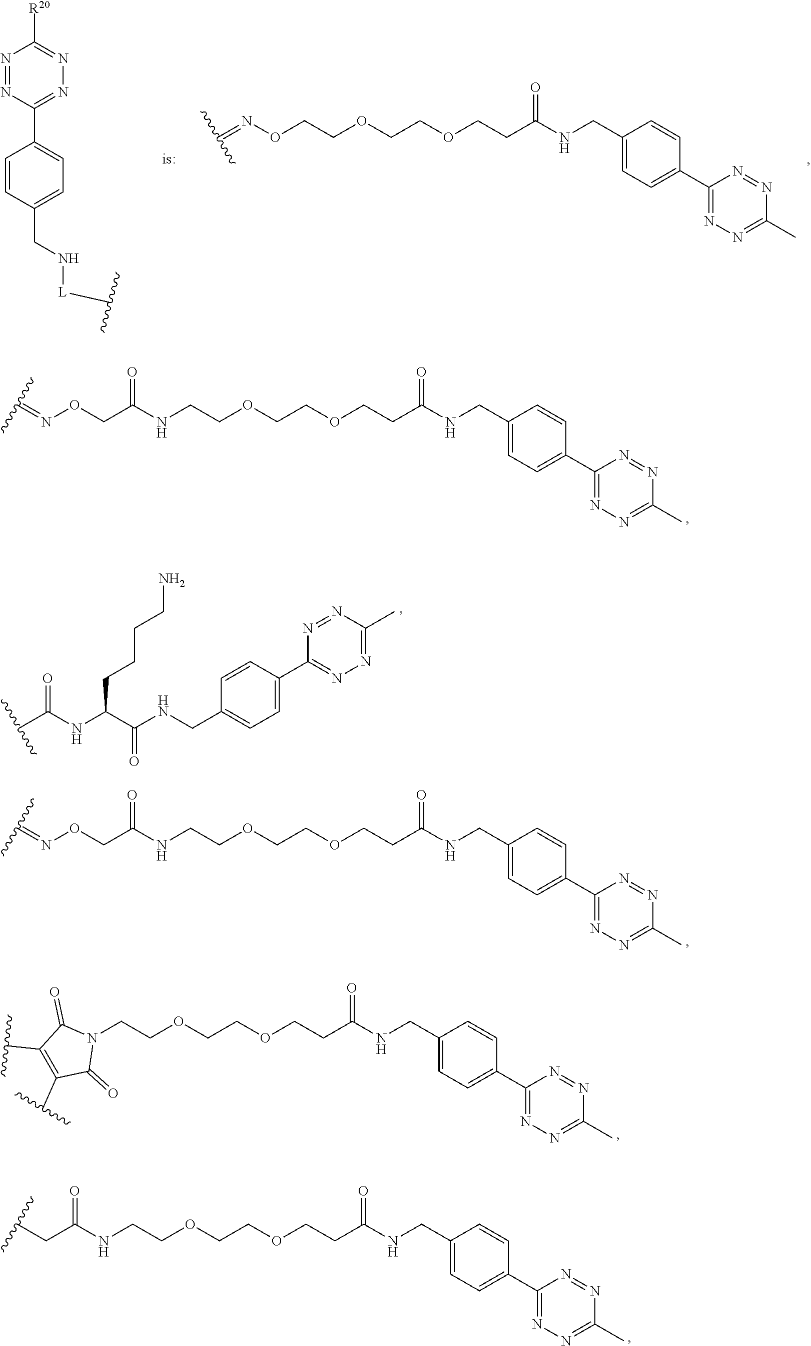

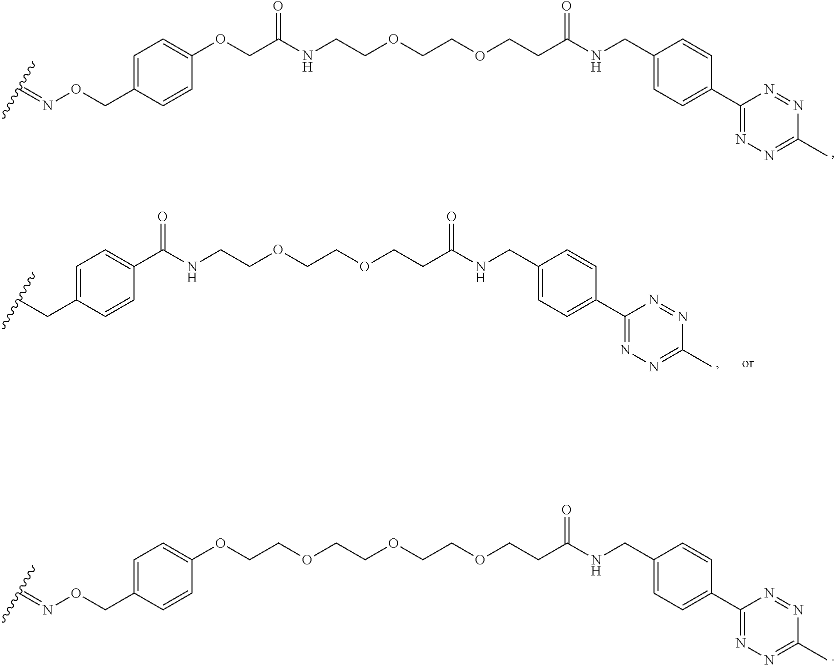 [SMILES: *=NOCc1ccc(OCC(=O)NCCOCCOCCC(=O)NCc2ccc(-c3nnc(C)nn3)cc2)cc1 ; *Cc1ccc(C(=O)NCCOCCOCCC(=O)NCc2ccc(-c3nnc(C)nn3)cc2)cc1 ; *=NOCc1ccc(OCCOCCOCCOCCC(=O)NCc2ccc(-c3nnc(C)nn3)cc2)cc1]

In some embodiments of Formula IIA, at least one of:

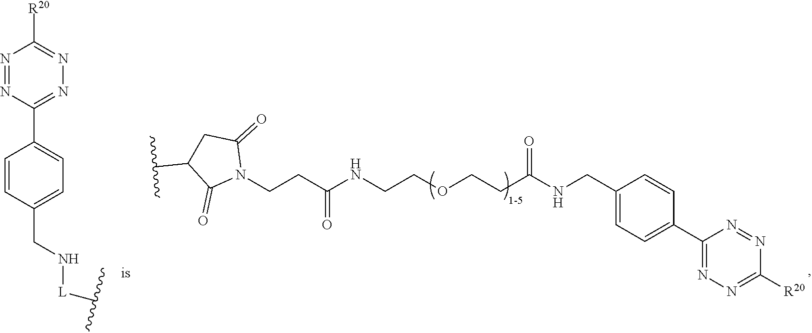

where R²⁰ is as defined herein.

In some embodiments of Formula IIA, at least one of:

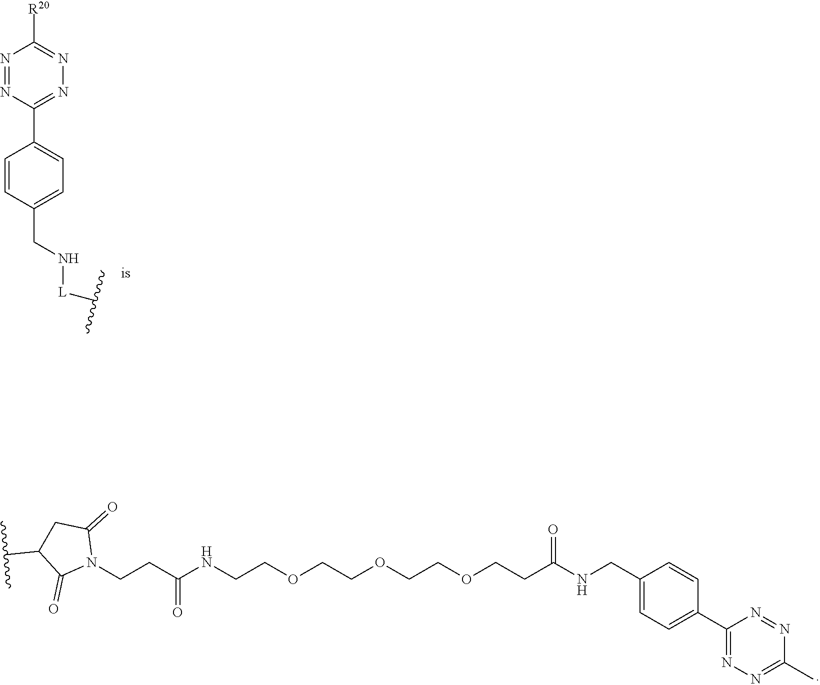

In some embodiments of Formula IIA, at least one of:

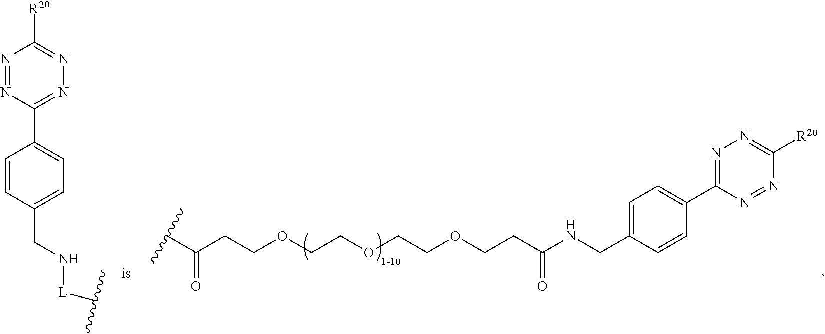

where R²⁰ is as defined herein.

In some embodiments of Formula IIA, at least one of:

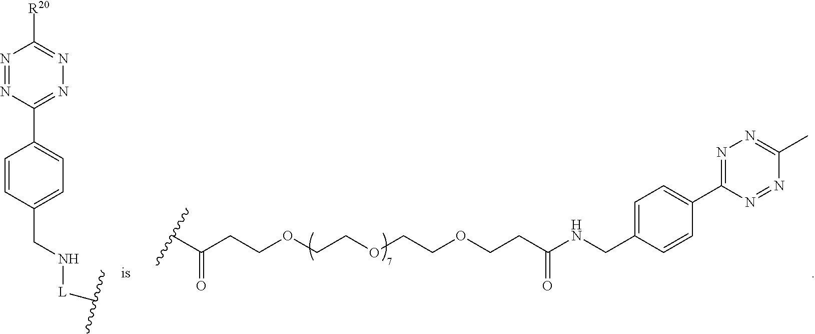

In some embodiments, p is 1-12. In some embodiments, X is an antibody. In some embodiments, p is 1-6, or 5-6. 2. In some embodiments, p is 1-16, or 1-8, or 1-7, or 1-6, or 1-5, or 1-4, or 1-3, or 1-2. In some embodiments, X is an antibody fragment moiety (e.g., Fab).

In one embodiment, the tetrazine activator is of Formula V:

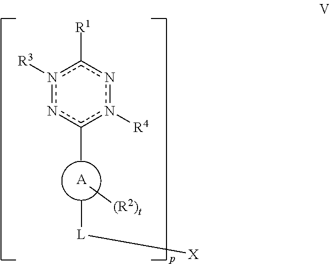

wherein:
ring A is aryl, cycloalkyl, heterocyclyl, or heteroaryl;
the dotted lines represent additional bonds to form a tetrazine when R³ and R⁴ are both absent, or a dihydrotetrazine when R³ and R⁴ are both present; provided that when ring A is aryl, then R³ and R⁴ are both present;
X is a biocompatible support, antibody, or antibody fragment moiety;
p is 1-150;
L, at each occurrence, is independently a linker;
R¹, at each occurrence, is independently selected from the group consisting of hydrogen, halo, cyano, nitro, alkyl, alkenyl, alkynyl, haloalkyl, heteroalkyl, aryl, heteroaryl, heterocyclyl, cycloalkyl, OR′, SR′, C(═O)R′, C(═S)R′, OC(═O)R′″, SC(═O)R″, OC(═S)R′″, SC(═S)R′″, S(═O)R′, S(═O)₂R′″, S(═O)₂NR′R″, C(═O)O—R′, C(═O)S—R′, C(═S)OR′, C(═S)SR′, C(═O)NR′R″, C(═S)NR′R″, NR′R″, NR′C(═O)R″, NR′C(═S)R″, NR′C(═O)OR″, NR′C(═S)OR″, NR′C(═O)SR″, NR′C(═S)SR″, OC(═O)NR′R″, SC(═O)NR′R″, OC(═S)R′R′″, SC(═S)R′R″, NR′C(═O)NR″R″, and NR′C(═S)NR″R″; wherein each alkyl, alkenyl, alkynyl, haloalkyl, heteroalkyl, aryl, heteroaryl, heterocyclyl, or cycloalkyl is optionally substituted with one to three Z¹;
R², at each occurrence, is independently halo, cyano, nitro, hydroxy, alkyl, haloalkyl, alkenyl, alkynyl, alkoxy, haloalkoxy, heteroalkyl, aryl, heteroaryl, heterocyclyl, cycloalkyl, —C(═O)-alkyl, —C(═O)-haloalkyl, —C(═O)-alkenyl, —C(═O)-alkynyl, —C(═O)-alkoxy, —C(═O)-haloalkoxy, —C(═O)-heteroalkyl, —C(═O)-aryl, —C(═O)-heteroaryl, —C(═O)-heterocyclyl, or —C(═O)-cycloalkyl; wherein each alkyl, haloalkyl, alkenyl, alkynyl, alkoxy, haloalkoxy, heteroalkyl, aryl, heteroaryl, heterocyclyl, or cycloalkyl is optionally substituted with one to three Z¹;
R³ and R⁴ are both absent; or R³ and R⁴ are each independently hydrogen or a group capable of being removed after a triggering event;
each Z¹ is independently selected from halo, oxo, cyano, nitro, hydroxy, alkyl, haloalkyl, alkenyl, alkynyl, alkoxy, haloalkoxy, heteroalkyl, aryl, heteroaryl, heterocyclyl, cycloalkyl, OR′, SR′, C(═O)R′, C(═S)R′, OC(═O)R′″, SC(═O)R″, OC(═S)R′″, SC(═S)R′″, S(═O)R′, S(═O)₂R′″, S(═O)₂NR′R″, C(═O)O—R′, C(═O)S—R′, C(═S)O—R′, C(═S)S—R′, C(═O)NR′R″, C(═S)NR′R″, NR′R″, NR′C(═O)R″, NR′C(═S)R″, NR′C(═O)OR″, NR′C(═S)OR″, NR′C(═O)SR″, NR′C(═S)SR″, OC(═O)NR′R″, SC(═O)NR′R″, OC(═S)R′R′″, SC(═S)R′R″, NR′C(═O)NR″R″, and NR′C(═S)NR″R″;
R′ and R″, at each occurrence, are independently selected from hydrogen, aryl, and alkyl;
R′″, at each occurrence, is independently selected from aryl and alkyl; and
t, at each occurrence, is independently is 0, 1, 2, 3, or 4.

In some embodiments, the tetrazine activator is of Formula V:

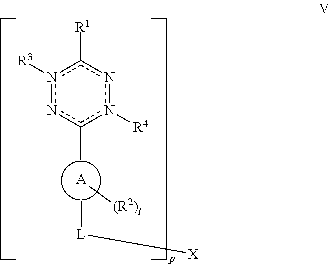

wherein:
ring A is cycloalkyl, heterocyclyl, or heteroaryl;
the dotted lines represent additional bonds to form a tetrazine when R³ and R⁴ are both absent, or a dihydrotetrazine when R³ and R⁴ are both present;
X is a biocompatible support, antibody, or antibody fragment moiety;
p is 1-150;
L, at each occurrence, is independently a linker;
R¹, at each occurrence, is independently selected from the group consisting of hydrogen, halo, cyano, nitro, alkyl, alkenyl, alkynyl, haloalkyl, heteroalkyl, aryl, heteroaryl, heterocyclyl, cycloalkyl, OR′, SR′, C(═O)R′, C(═S)R′, OC(═O)R′″, SC(═O)R″, OC(═S)R′″, SC(═S)R′″, S(═O)R′, S(═O)₂R′″, S(═O)₂NR′R″, C(═O)O—R′, C(═O)S—R′, C(═S)OR′, C(═S)SR′, C(═O)NR′R″, C(═S)NR′R″, NR′R″, NR′C(═O)R″, NR′C(═S)R″, NR′C(═O)OR″, NR′C(═S)OR″, NR′C(═O)SR″, NR′C(═S)SR″, OC(═O)NR′R″, SC(═O)NR′R″, OC(═S)R′R′″, SC(═S)R′R″, NR′C(═O)NR″R″, and NR′C(═S)NR″R″; wherein each alkyl, alkenyl, alkynyl, haloalkyl, heteroalkyl, aryl, heteroaryl, heterocyclyl, or cycloalkyl is optionally substituted with one to three Z¹;
R², at each occurrence, is independently halo, cyano, nitro, hydroxy, alkyl, haloalkyl, alkenyl, alkynyl, alkoxy, haloalkoxy, heteroalkyl, aryl, heteroaryl, heterocyclyl, cycloalkyl, —C(═O)-alkyl, —C(═O)-haloalkyl, —C(═O)-alkenyl, —C(═O)-alkynyl, —C(═O)-alkoxy, —C(═O)-haloalkoxy, —C(═O)-heteroalkyl, —C(═O)-aryl, —C(═O)-heteroaryl, —C(═O)-heterocyclyl, or —C(═O)-cycloalkyl; wherein each alkyl, haloalkyl, alkenyl, alkynyl, alkoxy, haloalkoxy, heteroalkyl, aryl, heteroaryl, heterocyclyl, or cycloalkyl is optionally substituted with one to three Z¹;
R³ and R⁴ are both absent; or
R³ is a group capable of being removed after a triggering event;
R⁴ is hydrogen or R³;
each Z¹ is independently selected from halo, oxo, cyano, nitro, hydroxy, alkyl, haloalkyl, alkenyl, alkynyl, alkoxy, haloalkoxy, heteroalkyl, aryl, heteroaryl, heterocyclyl, cycloalkyl, OR′, SR′, C(═O)R′, C(═S)R′, OC(═O)R′″, SC(═O)R″, OC(═S)R′″, SC(═S)R′″, S(═O)R′, S(═O)₂R′″, S(═O)₂NR′R″, C(═O)O—R′, C(═O)S—R′, C(═S)O—R′, C(═S)S—R′, C(═O)NR′R″, C(═S)NR′R″, NR′R″, NR′C(═O)R″, NR′C(═S)R″, NR′C(═O)OR″, NR′C(═S)OR″, NR′C(═O)SR″, NR′C(═S)SR″, OC(═O)NR′R″, SC(═O)NR′R″, OC(═S)R′R′″, SC(═S)R′R″, NR′C(═O)NR″R″, and NR′C(═S)NR″R″;
R′ and R″, at each occurrence, are independently selected from hydrogen, aryl, and alkyl;
R′″, at each occurrence, is independently selected from aryl and alkyl; and
t, at each occurrence, is independently is 0, 1, 2, 3, or 4.

In some embodiments, the tetrazine activator is of Formula VI:

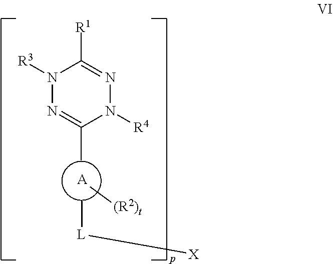

wherein each of R¹, R², R³, R⁴, ring A, L, p, t, and X are independently as defined herein.

In some embodiments, R⁴ is hydrogen.

In the targeting moieties described herein, R³ is a group capable of being removed after a triggering event. In some embodiments, the triggering event occurs in vivo. Once the triggering event occurs and R³ is removed, the dihydrotetrazine moiety is oxidized to provide a tetrazine as in Formula VII:

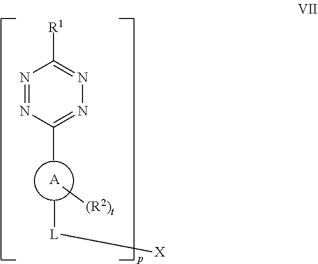

wherein each of R¹, R², ring A, L, p, t, and X are independently as defined herein.

The triggering event is initiated after administration of the tetrazine activator to the subject, and can be initiated by any means, such as internal means (e.g., via enzymatic cleavage of a functional group, optionally followed by a decomposition) or by external means (e.g., photocleavable linkers). In some embodiments, R³ comprises a targeting moiety, such as an antibody or antibody fragment as described herein.

In some embodiments, R³ comprises an amino acid sequence specific for cleavage by a protease or esterase.

In some embodiments, R³ comprises an amino acid sequence specific for cleavage by a protease as shown in Table 1A.

TABLE 1A

| Class of Proteases | Protease |
|---|---|
| Metalloproteinases | Meprin |
| | Neprilysin (CD10) |
| | PSMA |
| | BMP-1 |
| | A disintegrin and metalloproteinases (ADAMs) |
| | ADAM8 |
| | ADAM9 |
| | ADAM10 |
| | ADAM12 |
| | ADAM15 |
| | ADAM17 (TACE) |
| | ADAM19 |
| | ADAM28 (MDC-L) |
| | ADAM with thrombospondin motifs (ADAMTS) |
| | ADAMTS1 |
| | ADAMTS4 |
| | ADAMTS5 |
| | Matric Metalloproteinases (MMPs) |
| | MMP-1 (Collagenase 1) |
| | MMP-2 (Gelatinase A) |
| | MMP-3 (Stromelysin 1) |
| | MMP-7 (Matrilysin 1) |
| | MMP-8 (Collagenase 2) |
| | MMP-9 (Gelatinase B) |
| | MMP-10 (Stromelysin 2) |
| | MMP-11 (Stromelysin 3) |
| | MMP-12 (Macrophage elastase) |
| | MMP-13 (Collagenase 3) |
| | MMP-14 (MT1-MMP) |
| | MMP-15 (MT2-MMP) |
| | MMP-19 |
| | MMP-23 (CA-MMP) |
| | MMP-24 (MT5-MMP) |
| | MMP-26 (Matrilysin 2) |
| | MMP-27 (CMMP) |
| Cysteine Proteases | Legumain |
| | Cysteine Cathepsins |
| | Cathepsin B |
| | Cathepsin C |
| | Cathepsin K |
| | Cathepsin L |
| | Cathepsin S |
| | Cathepsin X |
| Aspartate Proteases | Cathepsin D |
| | Cathepsin E |
| | Secretase |
| Serine Proteases | Urokinase (uPA) |
| | Tissue-type plasminogen activator (tPA) |
| | Plasmin |
| | Thrombin |
| | Prostate-specific antigen (PSA, KLK3) |
| | Human neutrophil elastase (HNE) |
| | Elastase |
| | Tryptase |
| | Type II transmembrane serine proteases (TTSPs) |
| | DESC1 |
| | Hepsin (HPN) |
| | Matriptase |
| | Matriptase-2 |
| | TMPRSS2 |
| | TMPRSS3 |
| | TMPRSS4 (CAP2) |
| | Fibroblast Activation Protein (FAP) |
| | kallikrein-related peptidase (KLK family) |
| | KLK4 |
| | KLK5 |
| | KLK6 |
| | KLK7 |
| | KLK8 |
| | KLK10 |
| | KLK11 |
| | KLK13 |
| | KLK14 |

In some embodiments, R³ comprises an amino acid sequence specific for cleavage by a cathepsin, matrix metalloprotease (MMP), or PSMA. For example, in some embodiments, R³ comprises Val-Ala, Val-Cit, Ala-Ala, Phe-Lys, Lys-Lys, Phe-Arg, or Gly-Gly-Gly for cleavage by cathepsins. In some embodiments, R³ comprises Ac-γE-PLG-S(OBn)YL, or Ac-PLG-HofOrnL, where Hof is homophenylalanine and Orn is ornithine for cleavage by MMPs. In some embodiments, R³ comprises an amino acid sequence as shown Table 11B.

TABLE 1B

| PCM | Protease Acting | | |
|---|---|---|---|
| Designation | Upon Sequence | Exemplary Cleavage Sequence | Cleavage Sequences* |
| BSRS1 | MMP-2, 7, 9, 14, | LSGR ↓ SDN ↓ HSPLG ↓ | |
| | matriptase, uPA, | LAGS | |
| | legumain | | |
| BSRS2 | MMP-2, 7, 9, 14, | SPLG ↓ LAGSLSGR ↓ | |
| | matriptase, uPA, | SDN ↓ H | |
| | legumain | | |
| BSRS3 | MMP-2, 7, 9, 14, | SPLG ↓ LSGR ↓ SDN ↓ H | |
| | matriptase, uPA, | | |
| | legumain | | |
| BSRS4 | MMP-2, 7, 9, 14, | LAGR ↓ SDN ↓ HSPLG ↓ | |
| | matriptase, uPA, | LAGS | |
| | legumain | | |
| BSRS5 | MMP-2, 7, 9, 14, | LAGR ↓ SDN ↓ HVPLS ↓ | |
| | matriptase, uPA, | LSMG | |
| | legumain | | |
| BSRS6 | MMP-2, 7, 9, 14, | LAGR ↓ SDN ↓ HEPLE ↓ | |
| | matriptase, uPA, | LVAG | |
| | legumain | | |
| RS1 | MMP-2, 7, 9, 14 | SPLG ↓ LAGS | |
| RS2 | MMP-2, 7, 9, 14, | GPLG ↓ LAR ↓ G | |
| | matriptase, uPA, | | |
| | legumain | | |
| RS3 | Matriptase, uPA, | LSGR ↓ SDN ↓ H | |
| | legumain | | |
| RS4 | MMP-2, 14 | GTAH ↓ LMGG | |
| RS5 | MMP-14 | RIGS ↓ LRTA | |
| RS6 | MMP-14 | RIGA ↓ LRTA | |
| RS7 | MMP-14 | RIGW ↓ LRTA | |
| RS8 | MMP-14 | RIGN ↓ LRTA | |
| RS9 | MMP-14 | RIGF ↓ LRTA | |
| RS10 | MMP-14 | RIFF ↓ LRTA | |
| RS11 | MMP-14 | RILF ↓ LRTA | |
| RS12 | MMP-14 | RIYF ↓ LRTA | |
| RS13 | MMP-14 | RIQF ↓ LRTA | |
| RS14 | MMP-14 | EPAA ↓ LMAG | |
| RS15 | MMP-14 | EPAN ↓ LMAG | |
| RS16 | MMP-14 | EPAS ↓ LMAG | |
| RS17 | MMP-14 | EPFH ↓ LMAG | |
| RS18 | MMP-14 | EPWH ↓ LMAG | |
| RS19 | MMP-14 | EPRH ↓ LMAG | |
| RS20 | MMP-7 | VPLS ↓ LFMG | |
| RS21 | MMP-7 | VPLS ↓ LHMG | |
| RS22 | MMP-7 | VPLS ↓ LQAG | |
| RS23 | MMP-2, 7, 9, 14 | VPLS ↓ LTMG | |
| RS24 | MMP-2, 7, 9, 14, | VPLS ↓ LKMG | |
| | matriptase | | |
| RS25 | MMP-2, 7, 9, 14 | VPLS ↓ LSMG | |
| RS26 | MMP-7 | VPLS ↓ LNAG | |
| RS27 | MMP-7 | VPLS ↓ LLMG | |
| RS28 | MMP-7 | EPLE ↓ LPAG | |
| RS29 | MMP-2, 7, 9, 14 | EPLE ↓ LAAG | |
| RS30 | MMP-2, 7, 9 | EPLE ↓ LVAG | |
| RS31 | MMP-7 | EPLE ↓ LSAG | |
| RS32 | MMP-7 | EPLE ↓ LDAG | |
| RS33 | MMP-7 | EPLE ↓ LQAG | |
| RS34 | MMP-2, 7, 9, 14, | EPLE ↓ LRAG | |
| | matriptase | | |
| RS35 | MMP-7 | EPLE ↓ LKAG | |
| RS36 | MMP-2, 7, 9, 14 | EPLE ↓ LIAG | |
| RS37 | Elastase-2 | LGPV ↓ SGVP | —/—/—/VIAT/—/—/—/— |
| RS38 | Granzyme-B | VAGD ↓ SLEE | V/—/—/D/—/—/—/— |
| RS39 | MMP-12 | GPAG ↓ LGGA | G/PA/—/G/L/—/G/— |
| RS40 | MMP-13 | GPAG ↓ LRGA | G/P/—/G/L/—/GA/— |
| RS41 | MMP-17 | APLG ↓ LRLR | —/PS/—/—/LQ/—/LT/— |
| RS42 | MMP-20 | PALP ↓ LVAQ | |
| RS43 | TEV | ENLYFQ ↓ G | ENLYFQ/G/S |
| RS44 | Enterokinase | DDDK ↓ IVGG | DDDK/IVGG |
| RS45 | Protease 3C | LEVLFQ ↓ GP | LEVLFQ/GP |
| | (PreScission ™) | | |
| RS46 | Sortase A | LPKT ↓ GSES | L/P/KEAD/T/G/—/EKS/S |
| RS47 | Trypsin | K ↓ X** or R ↓ X | K/X or R/X |
| RS48 | Trypsin | R ↓ X** | SASRSA |
| RS49 | uPA | SGR ↓ SA | S/G/R/SRKA/AGSVR |
| RS50 | tPA | YGR ↓ SA | RYFLI/GA/R/RVAS/AG |
| RS51 | PSA | SSYY ↓ SG | S/S/FY/Y/S/G |
| RS52 | DESC1 | RRAR ↓ VVGG | R/RAL/ALY/R/AV/V/G/G |
| RS53 | Hepsin | RQLR ↓ VVGG | R/RQ/YL/R/V/V/G/G |
| RS54 | Matriptase-2 | RRAR ↓ VVGG | R/R/A/R/AV/V/G/G |
| RS55 | MT-SP1/Matriptase | RQAR ↓ VVGG | R/QR/A/R/AVY/V/G/G |
| RS56 | PSMA | N ↓ γN | N γ N |
| RS57 | Cathepsin C | GF ↓ FY | GP/FWR/**/— |
| RS58 | Cathepsin D | F ↓ IK | FL/IV/KE |
| RS59 | Cathepsin E | F ↓ K | FL/IV/KE |
| RS60 | Cathepsin F | WLR ↓ | WYRNle/L/RKQ |
| RS61 | Cathepsin K | KPR ↓ | KMGH/ILPNle/RKQ |
| RS62 | Cathepsin L | KFR ↓ | RKLnL/FYW/RKQ |
| RS63 | Cathepsin S | RVK ↓ | RPI/VLMnL/RKQ |
| RS64 | Cathepsin V/L2 | PWR ↓ | PNleR/WYF/RKQ |
| RS65 | MMP | PLG ↓ HofOrnL | |
| RS66 | MMP | EPCitF ↓ HofYL | |
| RS67 | MMP-2 | PQG ↓ IAGQ | |
| RS68 | MMP-2 | PQG ↓ IMelG | |
| RS69 | MMP-9 | AALG ↓ NvaP | |
| RS70 | MMP-9 | GPQG ↓ IAGQR | |
| RS71 | MMP-9 | SGKIPRT ↓ ATA | P/R/PSTRA/Hy/ST |
| RS72 | MMP-9 | SGPLF ↓ YSVTA | |
| RS73 | MMP-9 | PLR ↓ LSW | |
| RS74 | MMP-9 | GKGPRQ ↓ ITA | |
| RS75 | MMP-9 | SGRR ↓ LIHHT | S/G/R/R/L/IL |
| RS76 | MMP-9 | SGQPHY ↓ LTTA | |
| RS77 | MMP-9 | SG ↓ LKALM | |
| RS78 | MMP-9 | SGFGSRY ↓ LTA | |
| RS79 | MMP-9 | SGLRPAK ↓ STA | |
| RS80 | MMP-9 | LGP ↓ STST | |
| RS81 | MMP-9 | PQG ↓ VR | |
| RS82 | MMP-9 | PSG ↓ LP | P/S/G/L/HyP |
| RS83 | MMP-9 | PAG ↓ VQ | |
| RS84 | MMP-9 | PSG ↓ RD | |
| RS85 | MMP-9 | PPG ↓ IV | P/PG/G/Hy/HyR |
| RS86 | MMP-9 | PEN ↓ FF | |
| RS87 | MMP-9 | PLK ↓ LM | |
| RS88 | MMP-9 | PGA ↓ YH | |
| RS89 | MMP-9 | AIH ↓ IQ | |
| RS90 | MMP-9 | HFF ↓ KN | |
| RS91 | MMP-9 | GLS ↓ LS | |
| RS92 | MMP-9 | ASD ↓ YK | |
| RS93 | MMP-2, MMP-9 | GPLG ↓ MLSQ | P/Hy/G/Hy/HyWR |
| RS94 | MMP-2, MMP-9 | CG ↓ LDD | |
| RS95 | MMP-2, MMP-9, | GPQG ↓ IWGQ | |
| | MT1-MMP | | |
| RS96 | MMP-7 | RPLA ↓ LWRS | |
| RS97 | MMP-7 | GPLG ↓ LARK | |
| RS98 | Hk2 | GKAFR ↓ RL | |
| RS99 | MMP-9, uPA | RPSA ↓ SRSA | |
| RS100 | MMP-2 | PLGLDpaAR | |
| RS101 | MMP-9 | | P/LMVQChaHofNva/G/ |
| | | | LIYSFC/ST |
| RS102 | MMP-9 | PChaG ↓ SmcHA | P/LCha/G/LSmc/HW/A |
| RS103 | MMP-13, MMP-8 | PChaGNvaHAdF | |
| RS104 | ADAM10 | PTASA ↓ LKG | P/T/A/AS/A/LFYQ/KRTI/ |
| | | | GAS |
| RS105 | ADAM17 | PRPAA ↓ VKGT | P/HR/P/AS/A/VIL/KRTVI/ |
| | | | GST/TP |
| RS106 | Cathepsin B | V ↓ Cit | |
| RS107 | Cathepsin B | F ↓ K | |
| RS108 | Elastase | AA ↓ PV | |
| RS109 | Cathepsin D | GPIC ↓ FRLG | |
| RS110 | Plasmin | A ↓ FK | |
| RS111 | Legumain | AAN ↓ L | |
| RS112 | Legumain | PTN ↓ | PTAWS/TPASI/N |
| RS113 | Meprin | | ED/GTAV/— |
| RS114 | Meprin A | F ↓ SPFR | SFAMTY// |
| | | | SFAMTY/P/PVIGA/— |
| RS115 | Meprin B | E ↓ EEAY | DE/DE/YEFDG/PVIGA/— |
| RS116 | Neprilysin | _{β}-AIA ↓ L | _{β}-A/LI/A/L |
| RS117 | ADAMTS4 | E ↓ VQRKTGT | E/AFVLMY/(—)/ |
| | | | RK/— — (—)/ST |
| RS118 | ADAMTS4 | DVQE ↓ FRGVTAVIR | |
| RS119 | ADAMTS4 | HNE ↓ FRQRETYMVF | |
| RS120 | ADAMTS4 | KEEE ↓ GLGS | |
| RS121 | ADAMTS4 | GELE ↓ GRGT | |
| RS122 | ADAMTS4 | NITEGE ↓ ARGS | |
| RS123 | ADAMTS4 | TAQE ↓ AGEG | |
| RS124 | ADAMTS4 | VSQE ↓ LGQR | |
| RS125 | ADAMTS4 | PTAQE ↓ AGE | |

↓ indicates cleavage site
Special amino acid abbreviation:
Cit: Citrilline; Cha: β-cyclohexylalanine; Hof: homophenylalanine; Nva: aminosuberic acid; Dpa: D-phenylalanine; Nle: Norleucine; Smc: S-methylcysteine
*the listing of multiple amino acids before, between, or after a slash indicate alternative amino acids that can be substituted at the position;
—indicates that any amino acid may be substituted for the corresponding amino acid indicated in the middle column
**x is any L-amino acid other than proline
Hy is any hydrophobic L-amino acid
γ indicates that bond is a gamma carboxy linkage

Additional cleavable groups are described in Choi, et al., Theranostics. 2012; 2(2): 156-178, in which Table 2 is hereby incorporated by reference.

In some embodiments, R³ is photolabile. In some embodiments, the photolabile group is labile, or decomposes, with exposure to light at a wavelength matched to the absorbance profile of the photolabile group.

In some embodiments, R³ is

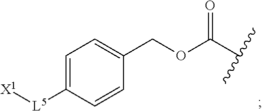

L⁵ is a direct bond or linker; and
X¹ is —NO₂, an optionally substituted sugar moiety, or an optionally substituted peptide unit comprising one or more natural or unnatural amino acids.

In some embodiments, at least one of the moiety:

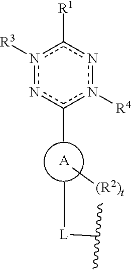

is represented by a formula selected from:

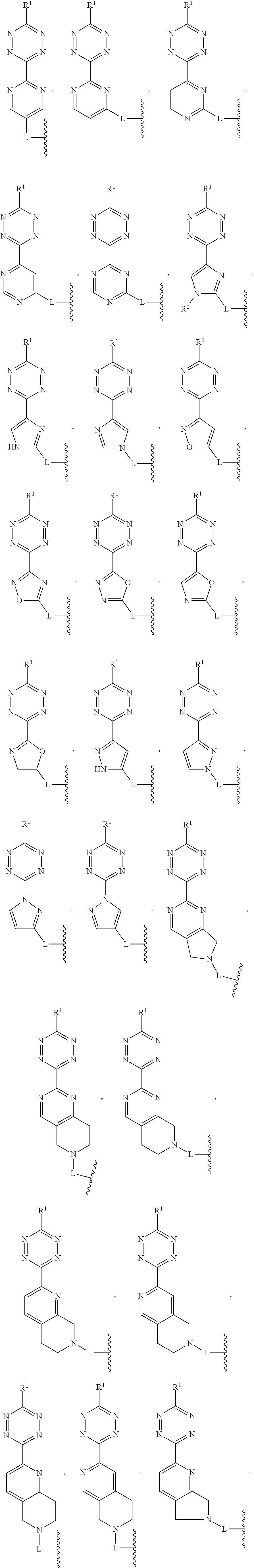

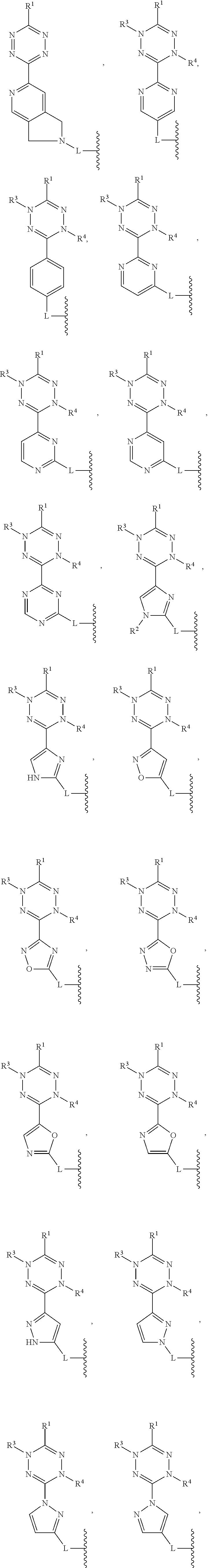

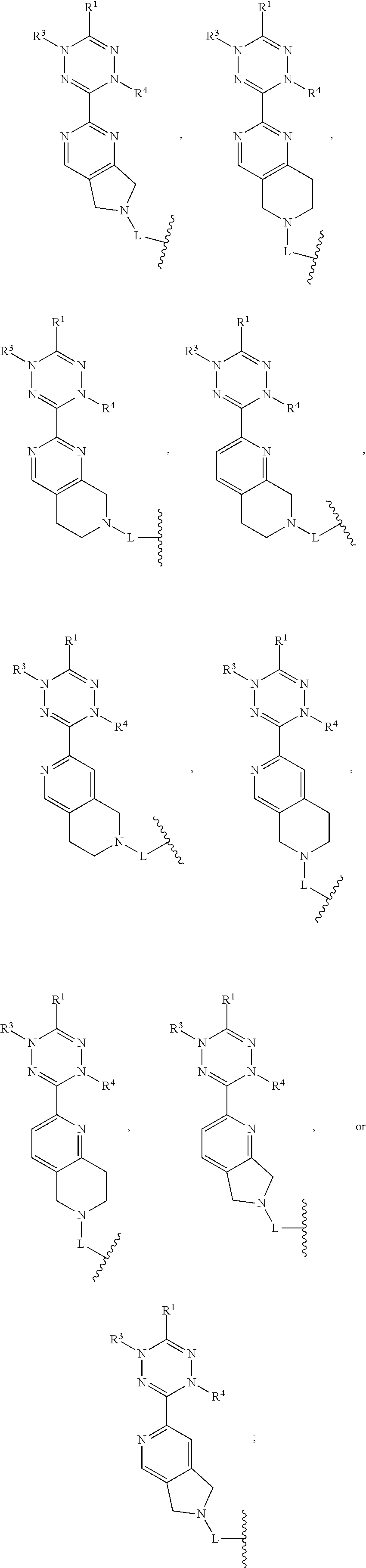

wherein each of R¹, R², R³, and R⁴ are independently as defined herein, and optionally the ring A portion may be substituted with one or more R² moieties.

In some embodiments, at least one of the moiety:

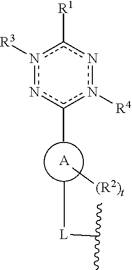

is represented by a formula selected from:

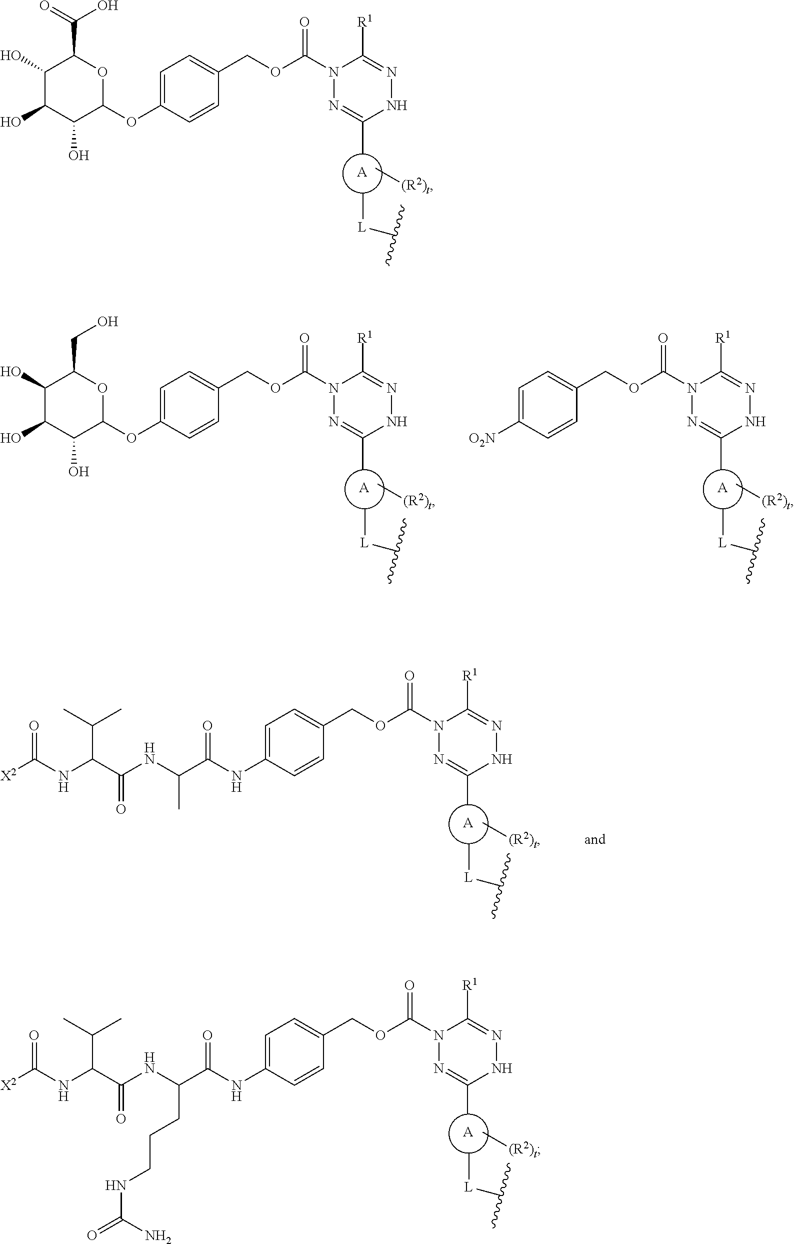

wherein X² is alkyl (e.g., methyl) optionally substituted with a PEG, an amino acid, ester, amide, amine, —C(O)OH, —SO₂, —SO₃, —PO₃, —PO₄, or other solubility enhancing substituent; and each of L, ring A, R¹, R², t, p, and X are independently as defined herein.

In some embodiments, ring A is cycloalkyl. In some embodiments, ring A is heterocyclyl. In some embodiments, ring A is heteroaryl. In some embodiments, ring A is aryl.

In some embodiments, ring A is pyrimidinyl, triazinyl, oxazolyl, isoxazole, imidazolyl, oxadiazolyl, 6,7-dihydro-5H-pyrrolo[3,4-d]pyrimidinyl, 5,6,7,8-tetrahydropyrido[4,3-d]pyrimidinyl, or 5,6,7,8-tetrahydropyrido[3,4-d]pyrimidinyl.

In some embodiments, ring A is phenyl.

In some embodiments, R¹, at each occurrence, is independently hydrogen, alkyl, alkenyl, alkynyl, haloalkyl, heteroalkyl, aryl, heteroaryl, heterocyclyl, or cycloalkyl; wherein each alkyl, alkenyl, alkynyl, haloalkyl, heteroalkyl, aryl, heteroaryl, heterocyclyl, or cycloalkyl is optionally substituted with one to three Z¹.

In some embodiments, R¹, at each occurrence, is independently hydrogen or alkyl optionally substituted with one to three Z¹.

In some embodiments, Z¹, at each occurrence, is independently selected from halo, hydroxy, alkoxy, and OC(═O)OR′.

In some embodiments, R², at each occurrence, is independently halo, cyano, nitro, hydroxy, alkyl, haloalkyl, alkenyl, alkynyl, alkoxy, haloalkoxy, heteroalkyl, aryl, heteroaryl, heterocyclyl, or cycloalkyl. In some embodiments, R², at each occurrence, is independently halo, alkyl, or haloalkyl. In some embodiments, R², at each occurrence, is independently halo or alkyl.

In some embodiments, t at each occurrence, is 0.

In some embodiments, the tetrazine activator is of Formula:

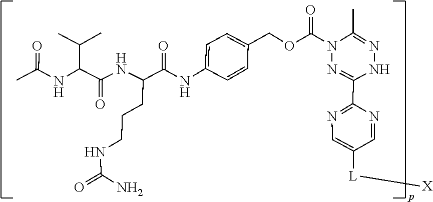

wherein p and X are each independently as defined herein. In some embodiments, the tetrazine activator is of Formula:

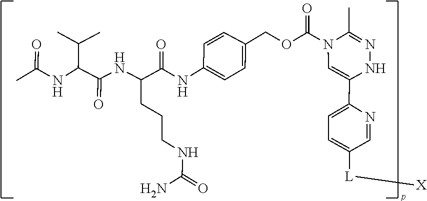

wherein p and X are each independently as defined herein.

In some embodiments, X is a biocompatible support.

In some embodiments, ring A is other than pyridyl. In some embodiments, ring A is other than aryl. In some embodiments, ring A is other than phenyl.

In some embodiments, X is a biocompatible support which comprises a particle, polymer, viscous or non-viscous liquid material, gel, hydrogel, a cross-linked polymer matrix, a metal, a ceramic, a plastic, a bone graft material, or a protein.

In some embodiments, X is a biocompatible support which comprises a polysaccharide hydrogel, alginate, cellulose, hyaluronic acid, chitosan, chitosin, chitin, hyaluronic acid, chondroitin sulfate, heparin, a suitable sugar-based biomaterial, a polyphosphazene, polyanhydride, polyacetal, poly(ortho ester), polyphosphoester, polycaprolactone, polyurethane, polylactide, polycarbonate, polyamide, polyether, a blend/composites/or co-polymer thereof, collagen, gelatin, elastin, an elastin-like polypeptide, albumin, fibrin, poly(gamma-glutamic acid), poly(L-lysine), poly(L-glutamic acid), or poly(aspartic acid). In some embodiments, X is a biocompatible support comprising hyaluronic acid with a molecular weight of about 5-25 kD, or 26-75 kD, or 76-200 kD, or >201 kD.

In some embodiments, X is an antibody or antibody fragment moiety.

### Antibody and Antibody Fragment Moieties

In certain embodiments, the targeting agent or X, is an antibody, or antibody fragment moiety, that targets one or more of CD25 (NCBI Gene ID 3559), CEA (NCBI Gene ID 634), CEACAM5 (NCBI Gene ID 1048), ASPH (NCBI Gene ID 444), EGFR (NCBI Gene ID 1956), EPCAM (NCBI Gene ID 4072), VEGFR (NCBI Gene ID 3791), PDGFR (NCBI Gene ID 5159), TROP2 (NCBI Gene ID 4070), Nectin4 (NCBI Gene ID 81607), PSMA (NCBI Gene ID 2346), BCMA (NCBI Gene ID 608), CD22 (NCBI Gene ID 933), CD20 (NCBI Gene ID 920), CD19 (NCBI Gene ID 930), CD79b (NCBI Gene ID 974), CD38 (NCBI Gene ID 952), CD45 (NCBI Gene ID 5788), Endoglin (NCBI Gene ID 2022), FGFR2 (NCBI Gene ID 14183), C4.4A (NCBI Gene ID 27076), Claudin-18.2 (NCBI Gene ID 51208), MMP9 (NCBI Gene ID 4318), Folate receptor (NCBI Gene ID 2348), DLL3 (NCBI Gene ID 10683), CD138 (NCBI Gene ID 6382), CD56 (NCBI Gene ID 4684), CD37 (NCBI Gene ID 951), CD74 (NCBI Gene ID 972), mesothelin (NCBI Gene ID 10232), IL-6R (NCBI Gene ID 3570), SLAMF7 (NCBI Gene ID 57823), BAFF (NCBI Gene ID 10673), MUC1 (NCBI Gene ID 4582), GPC3 (NCBI Gene ID 2719), HER2 (NCBI Gene ID 2064), HER3 (NCBI Gene ID 2065), CD30 (NCBI Gene ID 943), CD33 (NCBI Gene ID 945), CD123 (NCBI Gene ID 3563), GPNMB (NCBI Gene ID 10457), cMET (NCBI Gene ID 4233), CD142 (NCBI Gene ID 2152), NaPi2B (NCBI Gene ID 10568), GCC (NCBI Gene ID 2984), STEAP1 (NCBI Gene ID 26872), MUC16 (NCBI Gene ID 94025), CD70 (NCBI Gene ID 970), CD44 (NCBI Gene ID 960), (NCBI Gene ID), Antibody fragments (NCBI Gene ID), vWF (NCBI Gene ID 7450), TNF (NCBI Gene ID 7124), IL-6R (NCBI Gene ID 3570), BCMA (NCBI Gene ID 608), ADAMTS5 (NCBI Gene ID 11096), CX3CR1 (NCBI Gene ID 1524), CXCR4 (NCBI Gene ID 7852), TfR1 (NCBI Gene ID 7037), VEGFR (NCBI Gene ID 3791), or PSMA (NCBI Gene ID 2346).

In some embodiments, the targeting agent or X, is an antibody or antibody fragment moiety which targets CEA, CEACAM5, ASPH, EGFR, EPCAM, VEGFR, PDGFR, TROP2, Nectin4, PSMA, BCMA, HER2, CD25, CLDN4 (NCBI Gene ID 1364), TNC (NCBI Gene ID 3371), FN1 (NCBI Gene ID 2335), ITGAV (NCBI Gene ID 3685), TACSTD2 (NCBI Gene ID 4070), CD174 (NCBI Gene ID 2525), GPNMB (NCBI Gene ID 10457), GPC1 (NCBI Gene ID 2817), ITGB6 (NCBI Gene ID 3694), SEZ6 (NCBI Gene ID 124925), SLITRK6 (NCBI Gene ID 84189), NaPi-2b (NCBI Gene ID 20531), ZIP6 (NCBI Gene ID 25800), ROR1 (NCBI Gene ID 4919), or ROR2 (NCBI Gene ID 4920).

In certain embodiments, the targeting agent or X, is an antibody, or antibody fragment moiety, that targets one or more of CD25 (NCBI Gene ID 3559), CEA (NCBI Gene ID 634), CEACAM5 (NCBI Gene ID 1048), ASPH (NCBI Gene ID 444), EGFR (NCBI Gene ID 1956), EPCAM (NCBI Gene ID 4072), VEGFR (NCBI Gene ID 3791), PDGFR (NCBI Gene ID 5159), TROP2 (NCBI Gene ID 4070), Nectin4 (NCBI Gene ID 81607), PSMA (NCBI Gene ID 2346), BCMA (NCBI Gene ID 608), CD22 (NCBI Gene ID 933), CD20 (NCBI Gene ID 920), CD19 (NCBI Gene ID 930), CD79b (NCBI Gene ID 974), CD38 (NCBI Gene ID 952), CD45 (NCBI Gene ID 5788), Endoglin (NCBI Gene ID 2022), FGFR2 (NCBI Gene ID 14183), C4.4A (NCBI Gene ID 27076), Claudin-18.2 (NCBI Gene ID 51208), MMP9 (NCBI Gene ID 4318), Folate receptor (NCBI Gene ID 2348), DLL3 (NCBI Gene ID 10683), CD138 (NCBI Gene ID 6382), CD56 (NCBI Gene ID 4684), CD37 (NCBI Gene ID 951), CD74 (NCBI Gene ID 972), mesothelin (NCBI Gene ID 10232), IL-6R (NCBI Gene ID 3570), SLAMF7 (NCBI Gene ID 57823), BAFF (NCBI Gene ID 10673), MUC1 (NCBI Gene ID 4582), GPC3 (NCBI Gene ID 2719), HER2 (NCBI Gene ID 2064), HER3 (NCBI Gene ID 2065), CD30 (NCBI Gene ID 943), CD33 (NCBI Gene ID 945), CD123 (NCBI Gene ID 3563), GPNMB (NCBI Gene ID 10457), cMET (NCBI Gene ID 4233), CD142 (NCBI Gene ID 2152), NaPi2B (NCBI Gene ID 10568), GCC (NCBI Gene ID 2984), STEAP1 (NCBI Gene ID 26872), MUC16 (NCBI Gene ID 94025), CD70 (NCBI Gene ID 970), CD44 (NCBI Gene ID 960), (NCBI Gene ID), Antibody fragments (NCBI Gene ID), vWF (NCBI Gene ID 7450), TNF (NCBI Gene ID 7124), IL-6R (NCBI Gene ID 3570), BCMA (NCBI Gene ID 608), ADAMTS5 (NCBI Gene ID 11096), CX3CR1 (NCBI Gene ID 1524), CXCR4 (NCBI Gene ID 7852), TfR1 (NCBI Gene ID 7037), VEGFR (NCBI Gene ID 3791), PSMA (NCBI Gene ID 2346), ANTXR1 (NCBI Gene ID 84168), or FAP (NCBI Gene ID 2191).

In some embodiments, the targeting agent or X, is an antibody or antibody fragment moiety which targets CEA, CEACAM5, ASPH, EGFR, EPCAM, VEGFR, PDGFR, TROP2, Nectin4, PSMA, BCMA, HER2, CD25, ANTXR1, or FAP.

In some embodiments, the targeting agent or X, is an antibody or antibody fragment moiety that targets HER2, TROP2, Nectin-4, Claudin-18.2, MMP9, mesothelin, FN1, FAP, TNC, or ECM, EPCAM, CEA, or CEACAM5.

In some embodiments, the targeting agent or X, is an antibody or antibody fragment moiety which targets CEA, CEACAM5, ASPH, EGFR, EPCAM, VEGFR, PDGFR, TROP2, Nectin4, PSMA, BCMA, HER2, or CD25.

In certain embodiments, the targeting agent or X, is an antibody, or antibody fragment moiety, that targets CD25, such as daclizumab, RG6292, basiliximab, or HuMax-TAC, or an antibody fragment moiety derived therefrom.

In certain embodiments, the targeting agent or X, is an antibody, or antibody fragment moiety, that targets CEA, such as labetuzumab, 15-1-32, PR1A3, or cT84.66, or an antibody fragment moiety derived therefrom.

In certain embodiments, the targeting agent or X, is an antibody, or antibody fragment moiety, that targets CEACAM5, such as Tusamitiamab or CC4, or an antibody fragment moiety derived therefrom.

In certain embodiments, X is an antibody, or antibody fragment moiety, that targets ASPH, such as PAN-622, or an antibody fragment moiety derived therefrom.

In certain embodiments, the targeting agent or X, is an antibody, or antibody fragment moiety, that targets EGFR, such as cetuximab, necitumumab, nimotuzumab, matuzumab, AMG595, depatuxizumab, dapatuxizumab, duligotuzumab, futuximab, GC1118, imgatuzumab, panitumumab, alutumumab, tomuzotuximab, or laprituximab, or an antibody fragment moiety derived therefrom.

In certain embodiments, the targeting agent or X, is an antibody, or antibody fragment moiety, that targets EPCAM, such as oportuzumab, citatuzumab, tucotuzumab, catumaxomab, edrecolomab, or adecatumumab, or an antibody fragment moiety derived therefrom.

In certain embodiments, the targeting agent or X, is an antibody, or antibody fragment moiety, that targets VEGFR, such as ramucizumab, ramucirumab, or vulinacimab, or an antibody fragment moiety derived therefrom.

In certain embodiments, the targeting agent or X, is an antibody, or antibody fragment moiety, that targets PDGFR, such as olaratumab or ramucirumab, or an antibody fragment moiety derived therefrom.

In certain embodiments, the targeting agent or X, is an antibody, or antibody fragment moiety, that targets TROP2, such as Sacituzumab or Pr1E11, or an antibody fragment moiety derived therefrom.

In certain embodiments, the targeting agent or X, is an antibody, or antibody fragment moiety, that targets Nectin4, such as enfortumab, or an antibody fragment moiety derived therefrom.

In certain embodiments, the targeting agent or X, is an antibody, or antibody fragment moiety, that targets PSMA, such as J591 or MLN591, or an antibody fragment moiety derived therefrom.

In certain embodiments, the targeting agent or X, is an antibody, or antibody fragment moiety, that targets BCMA, such as belantamab, or an antibody fragment moiety derived therefrom.

In certain embodiments, the targeting agent or X, is an antibody, or antibody fragment moiety, that targets CD22, such as moxetumomab, inotuzumab, epratuzumab, or pinatuzumab, or an antibody fragment moiety derived therefrom.

In certain embodiments, the targeting agent or X, is an antibody, or antibody fragment moiety, that targets CD20, such as ublituximab, ofatumumab, rituximab, obinutuzumab, tositumomab, or ibritumomab, or an antibody fragment moiety derived therefrom.

In certain embodiments, the targeting agent or X, is an antibody, or antibody fragment moiety, that targets CD19, such as loncastuximab, XMAB-5574, MOR208, coltuximab, denintuzumab, taplitumomab, or MDX-1342, or an antibody fragment moiety derived therefrom.

In certain embodiments, the targeting agent or X, is an antibody, or antibody fragment moiety, that targets CD79b, such as polatuzumab, or an antibody fragment moiety derived therefrom.

In certain embodiments, the targeting agent or X, is an antibody, or antibody fragment moiety, that targets CD38, such as isatuximab, daratumumab, MOR202, or TAK-079, or an antibody fragment moiety derived therefrom.

In certain embodiments, the targeting agent or X, is an antibody, or antibody fragment moiety, that targets CD45, such as I-131-BC8, or Iomab-B, or an antibody fragment moiety derived therefrom.

In certain embodiments, the targeting agent or X, is an antibody, or antibody fragment moiety, that targets endoglin, such as carotuximab, or an antibody fragment moiety derived therefrom.

In certain embodiments, the targeting agent or X, is an antibody, or antibody fragment moiety, that targets FGFR2, such as bemarituzumab or aprutumab, or an antibody fragment moiety derived therefrom.

In certain embodiments, the targeting agent or X, is an antibody, or antibody fragment moiety, that targets C4.4A, such as lupartumab, or an antibody fragment moiety derived therefrom.

In certain embodiments, the targeting agent or X, is an antibody, or antibody fragment moiety, that targets Claudin-18.2, such as zolbetuximab, or claudiximab, or an antibody fragment moiety derived therefrom.

In certain embodiments, the targeting agent or X, is an antibody, or antibody fragment moiety, that targets MMP9, such as andecaliximab, or an antibody fragment moiety derived therefrom.

In certain embodiments, the targeting agent or X, is an antibody, or antibody fragment moiety, that targets folate receptor, such as mirvetuximab, farletuzumab, MORAb-202, MORAb-003, or SP8166, or an antibody fragment moiety derived therefrom.

In certain embodiments, the targeting agent or X, is an antibody, or antibody fragment moiety, that targets DLL3, such as rovalpituzumab, or an antibody fragment moiety derived therefrom.

In certain embodiments, the targeting agent or X, is an antibody, or antibody fragment moiety, that targets CD138, such as indatuximab, or an antibody fragment moiety derived therefrom.

In certain embodiments, the targeting agent or X, is an antibody, or antibody fragment moiety, that targets CD56, such as lorvotuzumab, promiximab, or an antibody fragment moiety derived therefrom.

In certain embodiments, the targeting agent or X, is an antibody, or antibody fragment moiety, that targets CD37, such as BI 836826, otlertuzumab, or naratuximab, or an antibody fragment moiety derived therefrom.

In certain embodiments, the targeting agent or X, is an antibody, or antibody fragment moiety, that targets CD74, such as milatuzumab, or an antibody fragment moiety derived therefrom.

In certain embodiments, the targeting agent or X, is an antibody, or antibody fragment moiety, that targets mesothelin, such as anetumab, amatuximab, or MMOT-0530A, or an antibody fragment moiety derived therefrom.

In certain embodiments, the targeting agent or X, is an antibody, or antibody fragment moiety, that targets IL-6R, such as tocilizumab or sarilumab, or an antibody fragment moiety derived therefrom.

In certain embodiments, the targeting agent or X, is an antibody, or antibody fragment moiety, that targets SLAMF7, such as elotuzumab, or an antibody fragment moiety derived therefrom.

In certain embodiments, the targeting agent or X, is an antibody, or antibody fragment moiety, that targets BAFF, such as belimumab, or an antibody fragment moiety derived therefrom.

In certain embodiments, the targeting agent or X, is an antibody, or antibody fragment moiety, that targets MUC1, such as KL-6, MY.1E12, hMUC1-1H7, TAB004, huC242, clivatuzumab, 8HuDS6, gatipotuzumab, AR20.5, or cantuzumab, or an antibody fragment moiety derived therefrom.

In certain embodiments, the targeting agent or X, is an antibody, or antibody fragment moiety, that targets GPC3, such as codrituzumab, ECT204, or MDX-1414, or an antibody fragment moiety derived therefrom.

In certain embodiments, the targeting agent or X, is an antibody, or antibody fragment moiety, that targets HER2, such as pertuzumab, trastuzumab, or margetuximab, or an antibody fragment moiety derived therefrom.

In certain embodiments, the targeting agent or X, is an antibody, or antibody fragment moiety, that targets HER3, such as patritumab, seribantumab, lumretuzumab, elgemtumab, AV-203, CDX-3379, or GSK284933, or an antibody fragment moiety derived therefrom.

In certain embodiments, the targeting agent or X, is an antibody, or antibody fragment moiety, that targets CD30, such as brentuximab, or an antibody fragment moiety derived therefrom.

In certain embodiments, the targeting agent or X, is an antibody, or antibody fragment moiety, that targets CD33, such as gemtuzumab, BI 835858, vadastuximab, or lintuzumab, or an antibody fragment moiety derived therefrom.

In certain embodiments, the targeting agent or X, is an antibody, or antibody fragment moiety, that targets CD123, such as KHK2823, taclotuzumab, or G4723A, or an antibody fragment moiety derived therefrom.

In certain embodiments, the targeting agent or X, is an antibody, or antibody fragment moiety, that targets GPNMB, such as glembatumumab, or an antibody fragment moiety derived therefrom.

In certain embodiments, the targeting agent or X, is an antibody, or antibody fragment moiety, that targets cMET, such as telisotuzumab, onartuzumab, or SAIT301, or an antibody fragment moiety derived therefrom.

In certain embodiments, the targeting agent or X, is an antibody, or antibody fragment moiety, that targets CD142, such as tisotumab, or an antibody fragment moiety derived therefrom.

In certain embodiments, the targeting agent or X, is an antibody, or antibody fragment moiety, that targets NaPi2B, such as lifastuzumab, or an antibody fragment moiety derived therefrom.

In certain embodiments, the targeting agent or X, is an antibody, or antibody fragment moiety, that targets GCC, such as indusatumab, or an antibody fragment moiety derived therefrom.

In certain embodiments, the targeting agent or X, is an antibody, or antibody fragment moiety, that targets STEAP1, such as vandortuzumab, or an antibody fragment moiety derived therefrom.

In certain embodiments, the targeting agent or X, is an antibody, or antibody fragment moiety, that targets MUC16, such as sofituzumab, or an antibody fragment moiety derived therefrom.

In certain embodiments, the targeting agent or X, is an antibody, or antibody fragment moiety, that targets CD70, such as vorsetuzumab, or an antibody fragment moiety derived therefrom.

In certain embodiments, the targeting agent or X, is an antibody, or antibody fragment moiety, that targets CD44, such as bivatuzumab, or an antibody fragment moiety derived therefrom.

In certain embodiments, the targeting agent or X, is an antibody, or antibody fragment moiety, that targets vWF, such as caplacizumab, or an antibody fragment moiety derived therefrom.

In certain embodiments, the targeting agent or X, is an antibody, or antibody fragment moiety, that targets TNF, such as ozoralizumab, V565, or PF-05230905, or an antibody fragment moiety derived therefrom.

In certain embodiments, the targeting agent or X, is an antibody, or antibody fragment moiety, that targets IL-6R, such as vobarilizumab, or an antibody fragment moiety derived therefrom.

In certain embodiments, the targeting agent or X, is an antibody, or antibody fragment moiety, that targets BCMA, such as LCAR-B38M, or an antibody fragment moiety derived therefrom.

In certain embodiments, the targeting agent or X, is an antibody, or antibody fragment moiety, that targets ADAMTS5, such as M6495, or an antibody fragment moiety derived therefrom.

In certain embodiments, the targeting agent or X, is an antibody, or antibody fragment moiety, that targets CX3CR1, such as BI 655088, or an antibody fragment moiety derived therefrom.

In certain embodiments, the targeting agent or X, is an antibody, or antibody fragment moiety, that targets CXCR4, such as AD-214 or ALX-0651, or an antibody fragment moiety derived therefrom.

In certain embodiments, the targeting agent or X, is an antibody, or antibody fragment moiety, that targets TfR1, such as TXB4, or an antibody fragment moiety derived therefrom.

In certain embodiments, the targeting agent or X, is an antibody, or antibody fragment moiety, that targets VEGFR, such as CDP791, or an antibody fragment moiety derived therefrom.

In certain embodiments, the targeting agent or X, is an antibody, or antibody fragment moiety, that targets PSMA, such as GY1, or an antibody fragment moiety derived therefrom.

In certain embodiments, the targeting agent or X, is an antibody, or antibody fragment moiety, that targets FN1, such as L19 or NJB2, or an antibody fragment moiety derived therefrom.

In certain embodiments, the targeting agent or X, is an antibody, or antibody fragment moiety, that targets FAP, such as F19, OMTX005 or sibrotuzumab, or an antibody fragment moiety derived therefrom.

In certain embodiments, the targeting agent or X, is an antibody, or antibody fragment moiety, that targets TNC, such as F16 or R6N or an antibody fragment moiety derived therefrom.

In some embodiments, the targeting agent or X, is an antibody.

In some embodiments, the antibody is daclizumab, RG6292, basiliximab, HuMax-TAC, labetuzumab, 15-1-32, PR1A3, cT84.66, tusamitiamab, CC4, PAN-622, cetuximab, necitumumab, nimotuzumab, matuzumab, AMG595, depatuxizumab, dapatuxizumab, duligotuzumab, Futuximab, GC1118, imgatuzumab, panitumumab, alutumumab, tomuzotuximab, laprituximab, oportuzumab, citatuzumab, tucotuzumab, catumaxomab, edrecolomab, adecatumumab, ramucizumab, ramucirumab, vulinacimab, olaratumab, ramucirumab, sacituzumab, Pr1E11, enfortumab, J591, MLN591, belantamab, moxetumomab, inotuzumab, epratuzumab, pinatuzumab, ublituximab, ofatumumab, rituximab, obinutuzumab, tositumomab, ibritumomab, loncastuximab, XMAB-5574, MOR208, coltuximab, denintuzumab, taplitumomab, MDX-1342, polatuzumab, Isatuximab, daratumumab, MOR202, TAK-079, I-131-BC8, Iomab-B, carotuximab, bemarituzumab, aprutumab, lupartumab, zolbetuximab, claudiximab, andecaliximab, mirvetuximab, farletuzumab, MORAb-202, MORAb-003, SP8166, rovalpituzumab, indatuximab, lorvotuzumab, promiximab, BI 836826, otlertuzumab, naratuximab, milatuzumab, anetumab, amatuximab, MMOT-0530A, sarilumab, elotuzumab, belimumab, KL-6, MY.1E12, hMUC1-1H7, TAB004, huC242, clivatuzumab, 8HuDS6, gatipotuzumab, AR20.5, cantuzumab, codrituzumab, ECT204, MDX-1414, pertuzumab, trastuzumab, margetuximab, patritumab, seribantumab, lumretuzumab, elgemtumab, AV-203, CDX-3379, GSK284933, brentuximab, gemtuzumab, BI 835858, vadastuximab, lintuzumab, KHK2823, taclotuzumab, G4723A, glembatumumab, telisotuzumab, onartuzumab, SAIT301, tisotumab, lifastuzumab, indusatumab, vandortuzumab, sofituzumab, vorsetuzumab, bivatuzumab, caplacizumab, ozoralizumab, V565, PF-05230905, vobarilizumab, LCAR-B38M, BI 655088, AD-214, ALX-0651, TXB4, CDP791, GY1, L19, NJB2, F19, OMTX005, sibrotuzumab, F16, or R6N.

In some embodiments, the targeting agent or X, is an antibody selected from atezolizumab, avelumab, bevacizumab, cemiplimab, cetuximab, daratumumab, dinutuximab, durvalumab, elotuzumab, ipilimumab, isatuximab, mogamulizumab, necitumumab, nivolumab, obinutuzumab, ofatumumab, olaratumab, panitumumab, pembrolizumab, pertuzumab, ramucirumab, rituximab, and trastuzumab.

In some embodiments, the targeting agent or X, is an antibody fragment moiety.

In some embodiments, the targeting agent or X, or the antibody fragment moiety is selected from the group consisting of a single-chain variable fragment (scFv), a divalent (or bivalent) single-chain variable fragment (di-scFvs, bi-scFvs), an antigen-binding fragment (Fab), a single-domain antibody (sdAb), a single-domain antibody (sdAb), an antigen-binding protein, a DotBody, an affibody, a DARPin, a DART, a TandAb, a diabody, a ribobody, a centyrin, a knottin, an affilin, an affimer, an alphabody, an anticalin, an atrimer, an avimer, a fynomer, a kunitz domain, an obody, a pronectin, a repebody, and a bicyclic peptide or a Humabody.

In some embodiments, the targeting agent or X, is an antibody fragment moiety selected from the group consisting of a single-chain variable fragment (scFv), a divalent (or bivalent) single-chain variable fragment (di-scFvs, bi-scFvs), an antigen-binding fragment (Fab), a single-domain antibody (sdAb), and a single-domain antibody (sdAb).

In some embodiments, the antibody fragment moiety is an antigen-binding protein a DotBody, affibody, DARPin, DART, TandAb, diabody, ribobody, centyrin, knottin, affilin, affimer, alphabody, anticalin, atrimer, avimer, fynomer, kunitz domain, obody, pronectin, repebody, bicyclic peptide or Humabody.

In some embodiments, the targeting agent or X, or the antibody fragment moiety is an antigen-binding fragment (Fab). The Fab is a region on an antibody that binds to antigens, and is comprised of one constant and one variable domain of each of the heavy and the light chain. In some embodiments, the Fab comprises four domains: VH, CH1, VL and CL1. In some embodiments, the Fab comprises 400-500 amino acids, or 440-480 amino acids. In some embodiments, the Fab has a molecular weight of about 50 kDa, or 40-55 kDa, or 45-50 kDa, or 45-55 kDa.

In some embodiments, the Fab of trastuzumab, enfortumab, brentuximab, sacituzumab, L19-binding to FN-1 (Gene ID 2335); F16-binding to TNC (Gene ID 3371), or NJB2 (ECM-targeting sequences).

In some embodiments, the antibody fragment moiety comprises one or more PEG units, which may enhance circulation life.

In some embodiments, the antibody fragment moiety is an antigen-binding protein. Antigen-binding proteins are proteins which are designed to be antibody-mimetics, exhibiting a high affinity and specificity for a given target. In some embodiments, the antigen-binding protein is a single-chain antigen-binding proteins are novel recombinant polypeptides, composed of an antibody variable light-chain amino acid sequence (VL) tethered to a variable heavy-chain sequence (VH) by a designed peptide that links the carboxyl terminus of the VL sequence to the amino terminus of the VH sequence.

In some embodiments, the antigen-binding protein is about 5-10 kDa, or about 7 kDa. In some embodiments, the antigen-binding protein is about are about 50-80, or 60-70, or 66 amino acids in length. In some embodiments, the antigen-binding protein comprises a cysteine only at the N- or C-terminus. In some embodiments, the antigen-binding protein comprises a cysteine only at the N-terminus. In some embodiments, the antigen-binding protein comprises a cysteine only at the C-terminus.

In some embodiments, the antibody fragment moiety is an antigen-binding protein that targets TNC, FN1, CLDN4, MMP9, EpCAM, ITGAV, CEA, CEACAM5, ASPH, EGFR, EPCAM, VEGFR, PDGFR, TROP2, Nectin4, PSMA, BCMA, HER2, or CD25. In some embodiments, the antibody fragment moiety is an antigen-binding protein that targets HER2. Antigen-binding proteins can be prepared and tested according to standard methods or purchased from commercial sources (e.g., Affilogic).

In some embodiments, the antibody fragment moiety is derived from daclizumab, RG6292, basiliximab, HuMax-TAC, labetuzumab, 15-1-32, PR1A3, cT84.66, tusamitiamab, CC4, PAN-622, cetuximab, necitumumab, nimotuzumab, matuzumab, AMG595, depatuxizumab, dapatuxizumab, duligotuzumab, futuximab, gc1118, imgatuzumab, panitumumab, alutumumab, tomuzotuximab, laprituximab, oportuzumab, citatuzumab, tucotuzumab, catumaxomab, edrecolomab, adecatumumab, ramucizumab, ramucirumab, vulinacimab, olaratumab, ramucirumab, sacituzumab, Pr1E1i, enfortumab, J591, MLN591, belantamab, moxetumomab, inotuzumab, epratuzumab, pinatuzumab, ublituximab, ofatumumab, rituximab, obinutuzumab, tositumomab, ibritumomab, loncastuximab, XMAB-5574, MOR208, coltuximab, denintuzumab, taplitumomab, MDX-1342, polatuzumab, isatuximab, daratumumab, MOR202, TAK-079, I-131-BC8, Iomab-B, carotuximab, bemarituzumab, aprutumab, lupartumab, zolbetuximab, claudiximab, andecaliximab, mirvetuximab, farletuzumab, MORAb-202, MORAb-003, SP8166, rovalpituzumab, indatuximab, lorvotuzumab, promiximab, BI 836826, otlertuzumab, naratuximab, milatuzumab, anetumab, amatuximab, MMOT-0530A, sarilumab, elotuzumab, belimumab, KL-6, MY.1E12, hMUC1-1H7, TAB004, huC242, clivatuzumab, 8HuDS6, gatipotuzumab, AR20.5, cantuzumab, codrituzumab, ECT204, MDX-1414, pertuzumab, trastuzumab, margetuximab, patritumab, seribantumab, lumretuzumab, elgemtumab, AV-203, CDX-3379, GSK284933, brentuximab, gemtuzumab, BI 835858, vadastuximab, lintuzumab, KHK2823, taclotuzumab, G4723A, glembatumumab, telisotuzumab, onartuzumab, sait301, tisotumab, lifastuzumab, indusatumab, vandortuzumab, sofituzumab, vorsetuzumab, bivatuzumab, caplacizumab, ozoralizumab, V565, PF-05230905, vobarilizumab, LCAR-B38M, BI 655088, AD-214, ALX-0651, TXB4, CDP791, GY1, L19, NJB2, F19, OMTX005, sibrotuzumab, F16 or R6N.

In some embodiments, X is an antibody fragment moiety derived from atezolizumab, avelumab, bevacizumab, cemiplimab, cetuximab, daratumumab, dinutuximab, durvalumab, elotuzumab, ipilimumab, isatuximab, mogamulizumab, necitumumab, nivolumab, obinutuzumab, ofatumumab, olaratumab, panitumumab, pembrolizumab, pertuzumab, ramucirumab, rituximab, or trastuzumab.

In certain embodiments, X is an antibody, or antibody fragment moiety, that targets vWF, such as Caplacizumab.

In certain embodiments, X is an antibody, or antibody fragment moiety, that targets TNF, such as Ozoralizumab, V565, or PF-05230905.

In certain embodiments, X is an antibody, or antibody fragment moiety, that targets IL-6R, such as Vobarilizumab.

In certain embodiments, X is an antibody, or antibody fragment moiety, that targets BCMA, such as LCAR-B38M.

In certain embodiments, X is an antibody, or antibody fragment moiety, that targets ADAMTS5, such as M6495.

In certain embodiments, X is an antibody, or antibody fragment moiety, that targets CX3CR1, such as BI 655088.

In certain embodiments, X is an antibody, or antibody fragment moiety, that targets CXCR4, such as AD-214 or ALX-0651.

In certain embodiments, X is an antibody, or antibody fragment moiety, that targets TfR1, such as TXB4.

In certain embodiments, X is an antibody, or antibody fragment moiety, that targets VEGFR, such as CDP791.

In certain embodiments, X is an antibody, or antibody fragment moiety, that targets PSMA, such as GY1.

In some embodiments, the antibody fragment moiety is caplacizumab, ozoralizumab, V565, PF-05230905, vobarilizumab, LCAR-B38M, M6495, BI 655088, AD-214, ALX-0651, TXB4, CDP791, or GY1.

In some embodiments, X further comprises an imaging contrast agent. In some embodiments, the imaging contrast agent is a protein.

### Linker Moieties

In some embodiments, L is bonded to X via a cystine or lysine residue on X.

In some embodiments, L is a non-cleavable linker.

In some embodiments, L is a cleavable linker.

In some embodiments, L comprises one or more amino acids.

In some embodiments, L comprises a polypeptide.

In some embodiments, L comprises one or more of a hydrazone, a hydrazide, a disulfide, a N-succinimidyl-4-(2-pyridyldithio)pentanoate (SPP), a N-succinimidyl-4-(2-pyridyldithio)butyrate (SPDB), a 4-(4′-acetylphenoxy)butanoic acid (AcBut), one or more linear or branched, natural or unnatural amino acid, a valine-citrulline (Val-Cit) moiety, or a phenylalanine-lysine (Phe-Lys) moiety.

In some embodiments, L comprises 1 to 100 linking atoms, from 1 to 50 linking atoms, or from 5 to 50 linking atoms, or from 10 to 50 linking atoms, or from 1 to 40 linking atoms, or from 1 to 30 linking atoms, or from 1 to 20 linking atoms, or from 1 to 10 linking atoms, or from 1 to 5 linking atoms, or from 5 to 30 linking atoms, or from 10 to 30 linking atoms, or from 5 to 40 linking atoms, or from 5 to 50 linking atoms, or from 10 to 50 linking atoms.

In some embodiments, L comprises one or more chain heteroatoms and one or more alkylene, alkenylene, alkynylene, arylene, heteroarylene, cycloalkylene or heterocycloalkylene moieties; wherein each alkylene, alkenylene, alkynylene, arylene, heteroarylene, cycloalkylene or heterocycloalkylene moiety, may be independently optionally substituted with one to five substituents independently selected from oxo, halo, C₁₋₄ alkyl, C₁₋₄ alkoxy, and C₁₋₄ haloalkyl.

In some embodiments, L is an alkylene linker optionally comprising one or more —O—, —S—, amine, ester, amide, carbamate, carbonate, thio-succinimide, or ketone functional groups.

In some embodiments, linker L is of the formula:

—Y¹⁰—(CHR¹³⁰)_{n′}—Y²⁰—(CHR¹⁴⁰)_{n″}—Y³⁰—(CHR¹⁵⁰)_{m″}—Y⁴⁰—
wherein:
each of Y¹⁰, Y²⁰, Y³⁰, and Y⁴⁰ are independently a bond, —NR¹¹⁰—, —O—, —S(O)₀₋₂—, —NR¹¹⁰C(O)—, —C(O)NR¹¹⁰—, —NR¹¹⁰S(O)₂—, —S(O)₂NR¹¹⁰—, —CR¹²⁰═N—NR¹¹⁰—, —NR¹¹⁰—N═CR¹²⁰—, —C(O)—, —OC(O)—, —OC(O)O—, —(CH₂CH₂O)₁₋₅—, —C(O)O—, alkylene, alkenylene, alkynylene, arylene, or heteroarylene; wherein each alkylene, alkenylene, alkynylene, arylene, or heteroarylene is independently optionally substituted with one to five substituents independently selected from oxo, halo, C₁₋₄ alkyl, C₁₋₄ alkoxy, and C₁₋₄ haloalkyl;
each R¹¹⁰ is independently hydrogen, C₁₋₄ alkyl, C₁₋₄ haloalkyl, aryl, heteroaryl, cycloalkyl, or heterocyclyl;
each R¹²⁰ is independently hydrogen, C₁₋₄ alkyl, C₁₋₄ haloalkyl, aryl, heteroaryl, cycloalkyl, or heterocyclyl;
each R¹³⁰ is independently hydrogen, C₁₋₄ alkyl, C₁₋₄ haloalkyl, aryl, heteroaryl, cycloalkyl, heterocyclyl or an amino acid side chain;
each R¹⁴⁰ is independently hydrogen, C₁₋₄ alkyl, C₁₋₄ haloalkyl, aryl, heteroaryl, cycloalkyl, heterocyclyl, or an amino acid side chain;
each R¹⁵⁰ is independently hydrogen, C₁₋₄ alkyl, C₁₋₄ haloalkyl, aryl, heteroaryl, cycloalkyl, heterocyclyl or an amino acid side chain; and
n′, n″, and m″ are each independently 0, 1, 2, 3, 4, 5, 6, 7, or 8.

In some embodiments, L is of the formula:

—Y¹⁰—(CH₂)_{n′}—Y²⁰—(CH₂)_{m″}—Y³⁰—
wherein:
each of Y¹⁰, Y²⁰, and Y³⁰ are independently a bond, —NR¹¹⁰—, —O—, —S(O)₀₋₂—, —NR¹¹⁰C(O)—, —C(O)NR¹¹⁰—, —NR¹¹⁰S(O)₂—, —S(O)₂NR¹¹⁰—, —CR¹²⁰═N—NR¹¹⁰—, —NR″ O—N═CR¹²⁰—, —C(O)—, —OC(O)—, —OC(O)O—, alkylene, alkenylene, alkynylene, arylene, heteroarylene, cycloalkylene or heterocycloalkylene; wherein each alkylene, alkenylene, alkynylene, arylene, heteroarylene, cycloalkylene or heterocycloalkylene is independently optionally substituted with one to five substituents independently selected from oxo, halo, C₁₋₄ alkyl, C₁₋₄ alkoxy, and C₁₋₄ haloalkyl;
each R¹¹⁰ is independently hydrogen, C₁₋₄ alkyl, C₁₋₄ haloalkyl, aryl, heteroaryl, cycloalkyl or heterocyclyl;
each R¹²⁰ is independently hydrogen, C₁₋₄ alkyl, C₁₋₄ haloalkyl, aryl, heteroaryl, cycloalkyl or heterocyclyl; and
n′ and m″ are each independently 0, 1, 2, 3, 4, 5, 6, 7, or 8.

In certain embodiments, each R¹¹⁰ is independently hydrogen, C₁₋₄ alkyl, C₁₋₄ haloalkyl, aryl, heteroaryl, cycloalkyl or heterocyclyl; and each R¹²⁰ is independently hydrogen, C₁₋₄ alkyl, C₁₋₄ haloalkyl, aryl, heteroaryl, cycloalkyl or heterocyclyl. In certain embodiments, the linker is not a bond.

The linker L may comprise one or more of polyethylene glycol (e.g., PEG having an average molecular weight of from 300 g/mol to 10,000 g/mol), ethylene-1,2-diylbis(methylcarbamate, an arylene (e.g., phenylene), ethylene-oxy, amine, ester, amide, carbamate, ketone (i.e., formyl), or carbonate.

In some embodiments, the linker comprises one or more of:

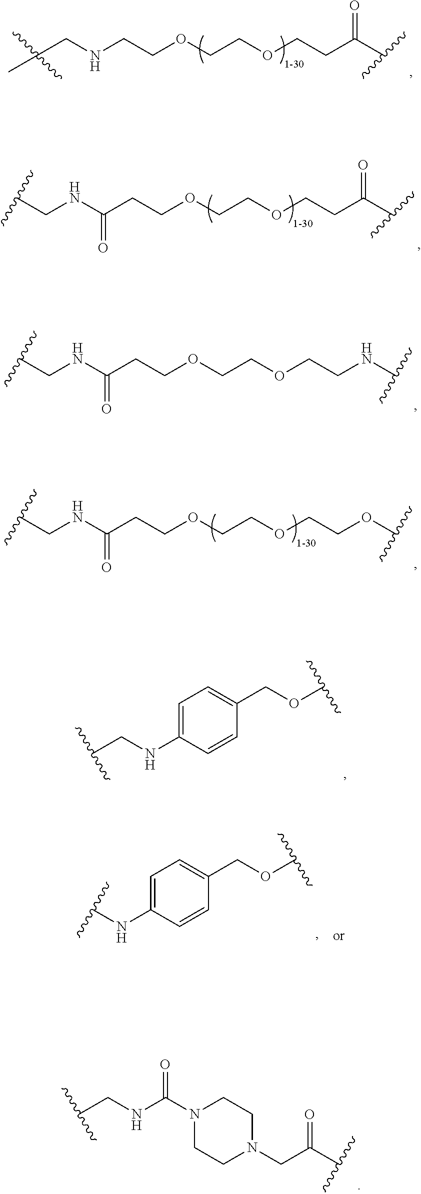

In some embodiments, the linker comprises one or more of:

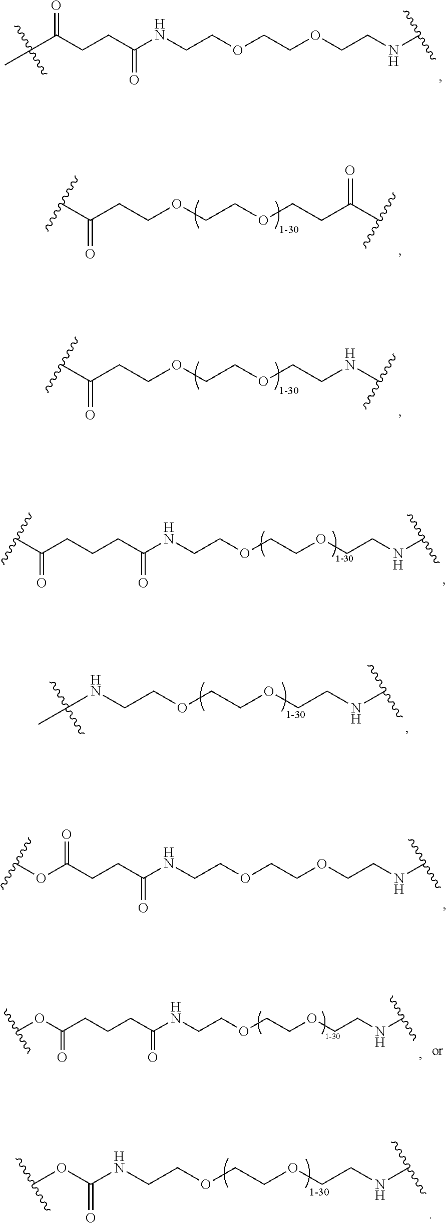

In some embodiments, the linker comprises one or more of:

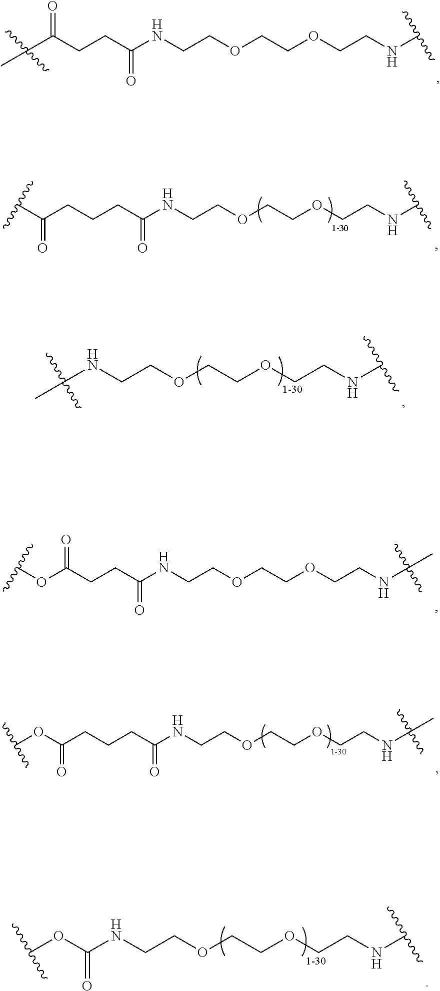

In some embodiments, the linker comprises one or more

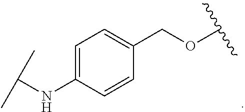 [SMILES: *OCc1ccc(NC(C)C)cc1]

In some embodiments, the linker comprises one or more

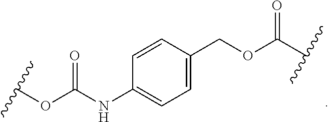 [SMILES: *OC(=O)Nc1ccc(COC(*)=O)cc1]

In some embodiments, the linker is, or comprises one or more:

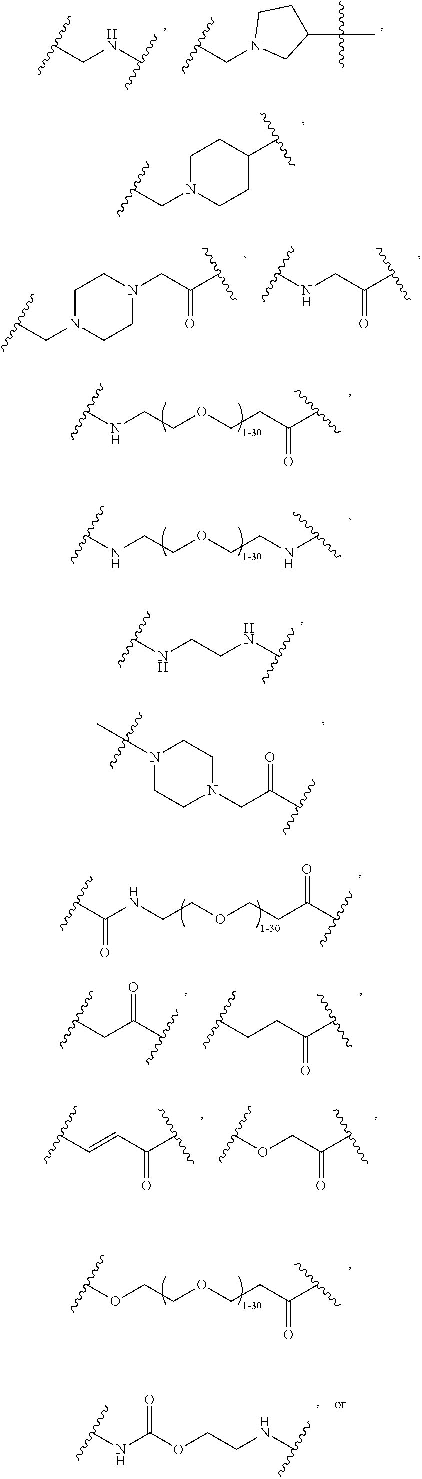

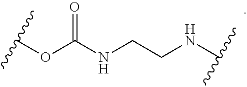 [SMILES: *NCCNC(=O)O*]

In some embodiments, the linker is, or comprises one or more:

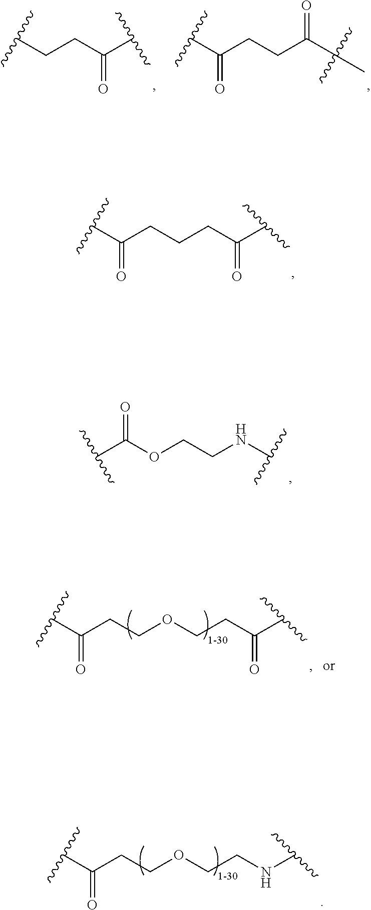

In some embodiments, the linker comprises one or more natural or unnatural amino acids, which may be referred to as a peptide linker. The linker may be a peptide linker made up of a carboxylic acyl unit, and one or more amino acids making up a protein or peptide sequence. The linker may also contain a self-immolating spacer which spaces the drug and the protein peptide sequence.

In some embodiments, the linker may be a peptide containing linker represented by “A-Y—Z—X²—W” in which “A” is the carboxylic acyl unit, “Y” and “Z” are each one or more natural or unnatural amino acids and together form a peptide sequence, and “X²” and “W” are optional additional linkers having from 1 to 50 linking atoms, or from 5 to 10 linking atoms, or from 1 to 10 linking atoms which spaces the peptide and the payload, D, or the bioorthogonal moiety. In certain embodiments, one or more of the amino acids in the peptide linker is N-methylated.

In some embodiments, Y may be at least one amino acid selected from the group consisting of alanine, valine, leucine, isoleucine, methionine, phenylalanine, tryptophan, and proline. In some embodiments Y may be at least one amino acid selected from the group consisting of phenylalanine, alanine, and valine.

In some embodiments, Z may be at least one amino acid selected from the group consisting of alanine, lysine, lysine protected with acetyl or formyl, arginine, arginine protected with tosyl or nitro groups, histidine, ornithine, ornithine protected with acetyl or formyl, and citrulline. In some embodiments Z may be at least one amino acid selected from the group consisting of alanine, lysine, and citrulline.

Exemplary Y—Z combinations include Valine-Citrulline; Valine-Alanine; and Alanine-Alanine.

In some embodiments, A is —OC(O)—.

In some embodiments, X² is —OC(O)—.

In some embodiments, W is —OC(O)—. In some embodiments, X² is absent and W is —OC(O)—.

In some embodiments, the moiety —X²—W comprises

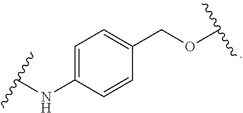 [SMILES: *Nc1ccc(CO*)cc1]

In some embodiments, the moiety —X² is

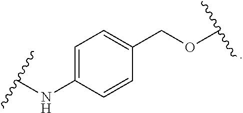 [SMILES: *Nc1ccc(CO*)cc1]

In some embodiments, —X²—W is

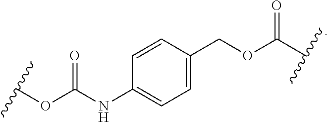 [SMILES: *OC(=O)Nc1ccc(COC(*)=O)cc1]

In some embodiments, —X²—W is

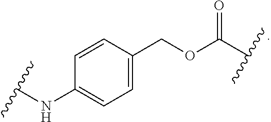 [SMILES: *Nc1ccc(COC(*)=O)cc1]

In some embodiments, the peptide linker is specifically tailored so that it will be selectively cleaved (e.g., enzymatically cleaved) releasing the drug, such as by one or more of the tumor-associated proteases.

In some embodiments, the peptide linker has a chain length of two to four amino acid residues (i.e., a di-, tri-, or tetra-peptide). It will be understood, however, that peptide linkers up to five, six, seven, or eight amino acid residues may also suitably be employed.

In some embodiments, the peptide linker is Phe-Lys, Val-Lys, Val-Ala, Ala-Ala, Phe-Phe-Lys, D-Phe-Phe-Lys, Gly-Phe-Lys, Ala-Lys, Val-Cit, Phe-Cit, Leu-Cit, Ile-Cit, Trp-Cit, Phe-Ala, Gly-Phe-Leu-Gly [SEQ ID NO: 1], Ala-Leu-Ala-Leu [SEQ ID NO: 2], Phe-N⁹-tosyl-Arg, or Phe-N⁹-Nitro-Arg. In certain embodiments, the peptide linker is Phe-Lys, Val-Lys, Val-Ala, Ala-Ala, Val-Val, Val-Cit, or D-Phe-L-Phe-Lys. In certain embodiments, the peptide linker is Val-Cit, Val-Ala, or Ala-Ala.

In some embodiments, the linker L is, or comprises one or more of:

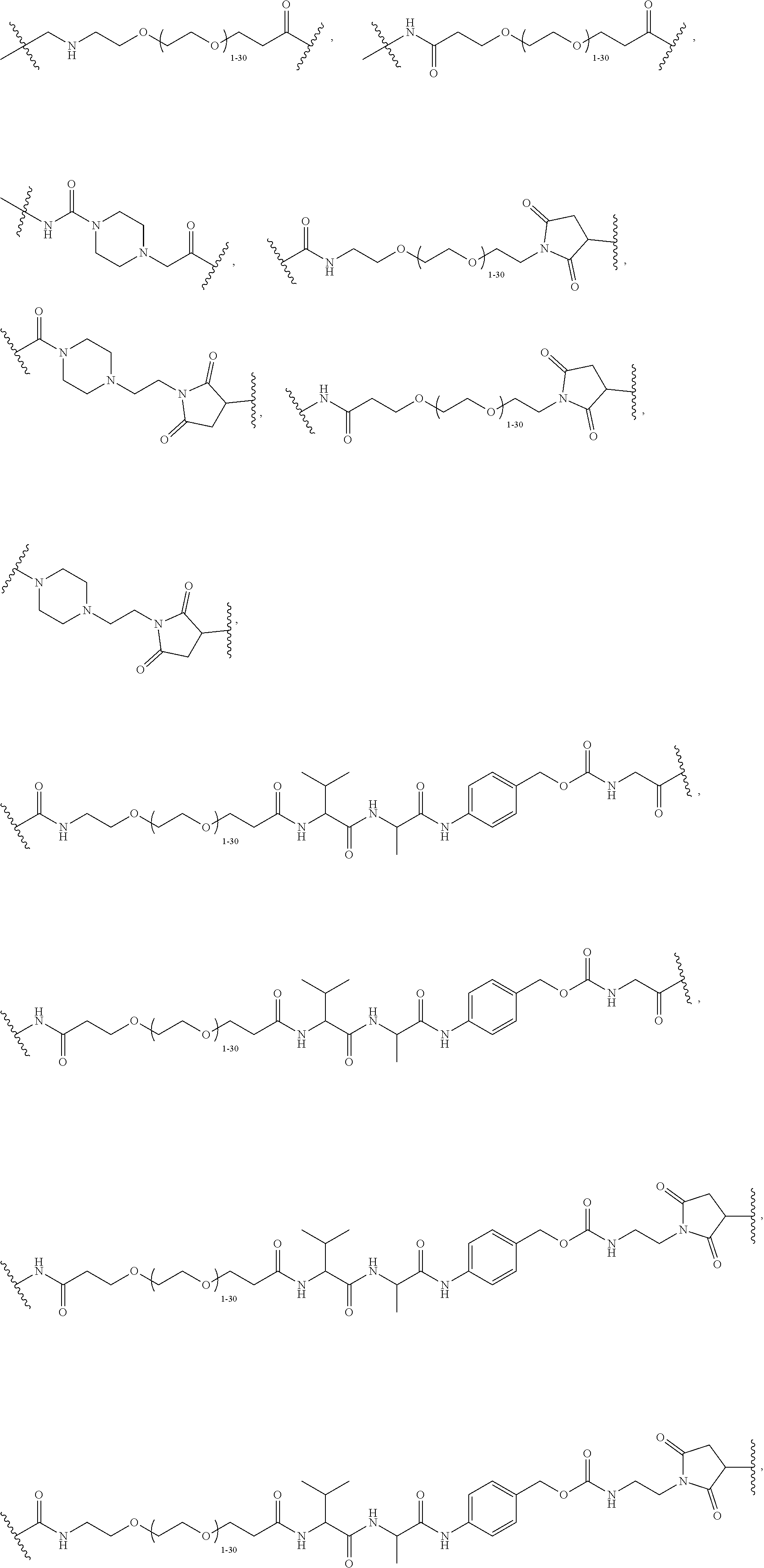

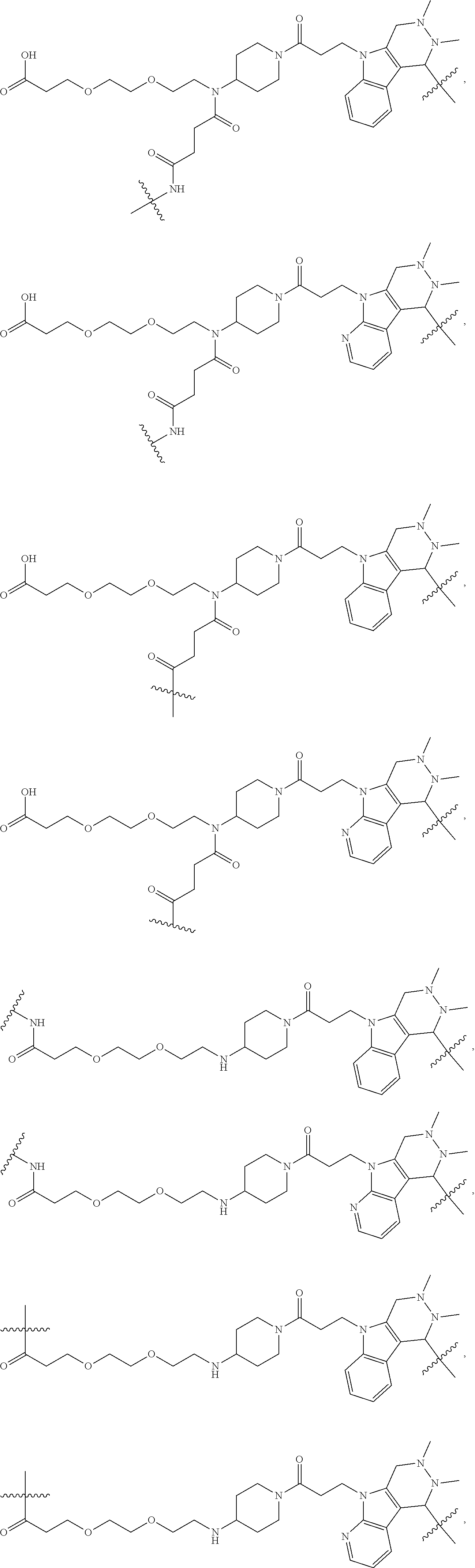

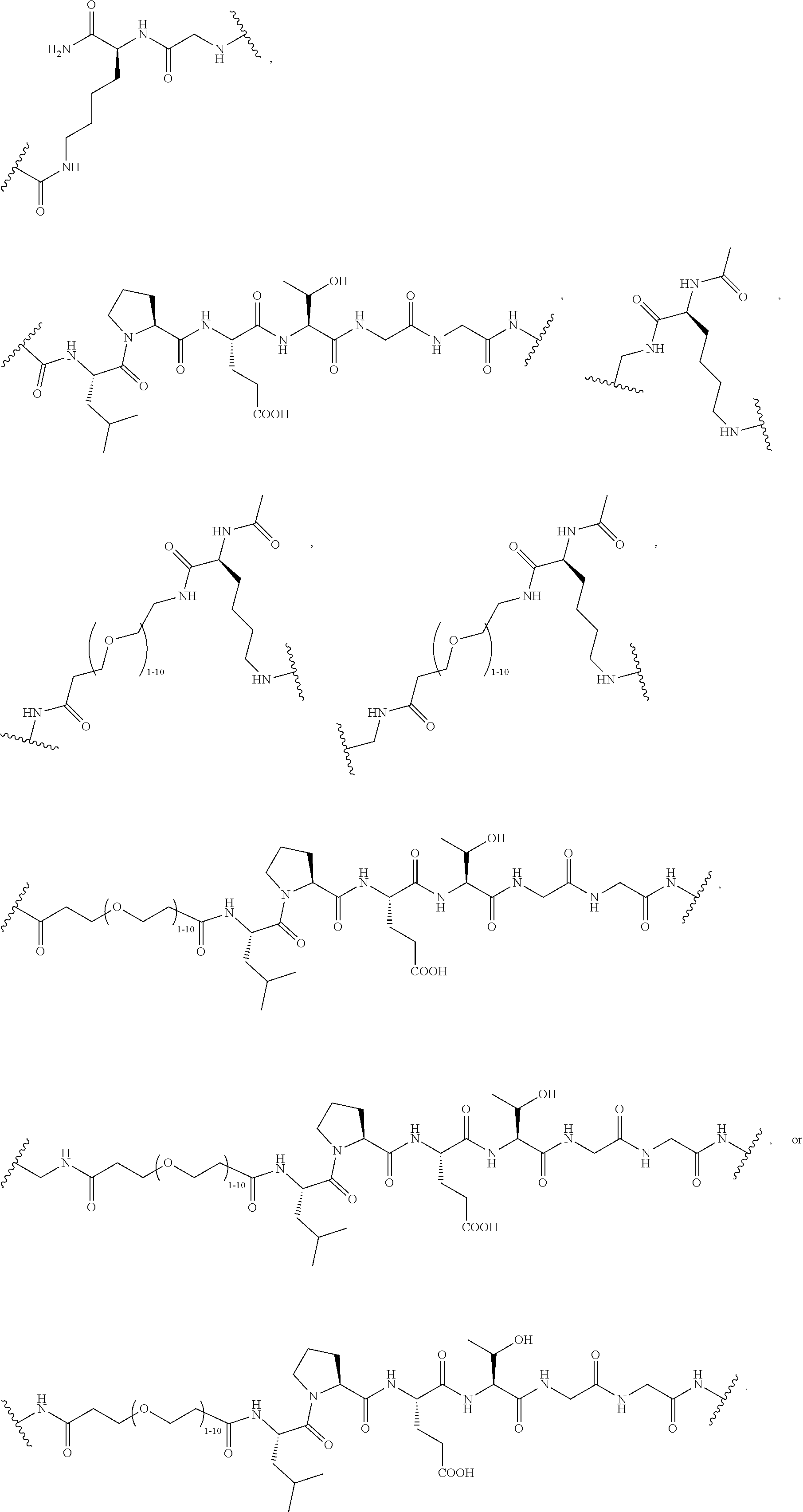

In some embodiments, the linker L comprises one or more of:

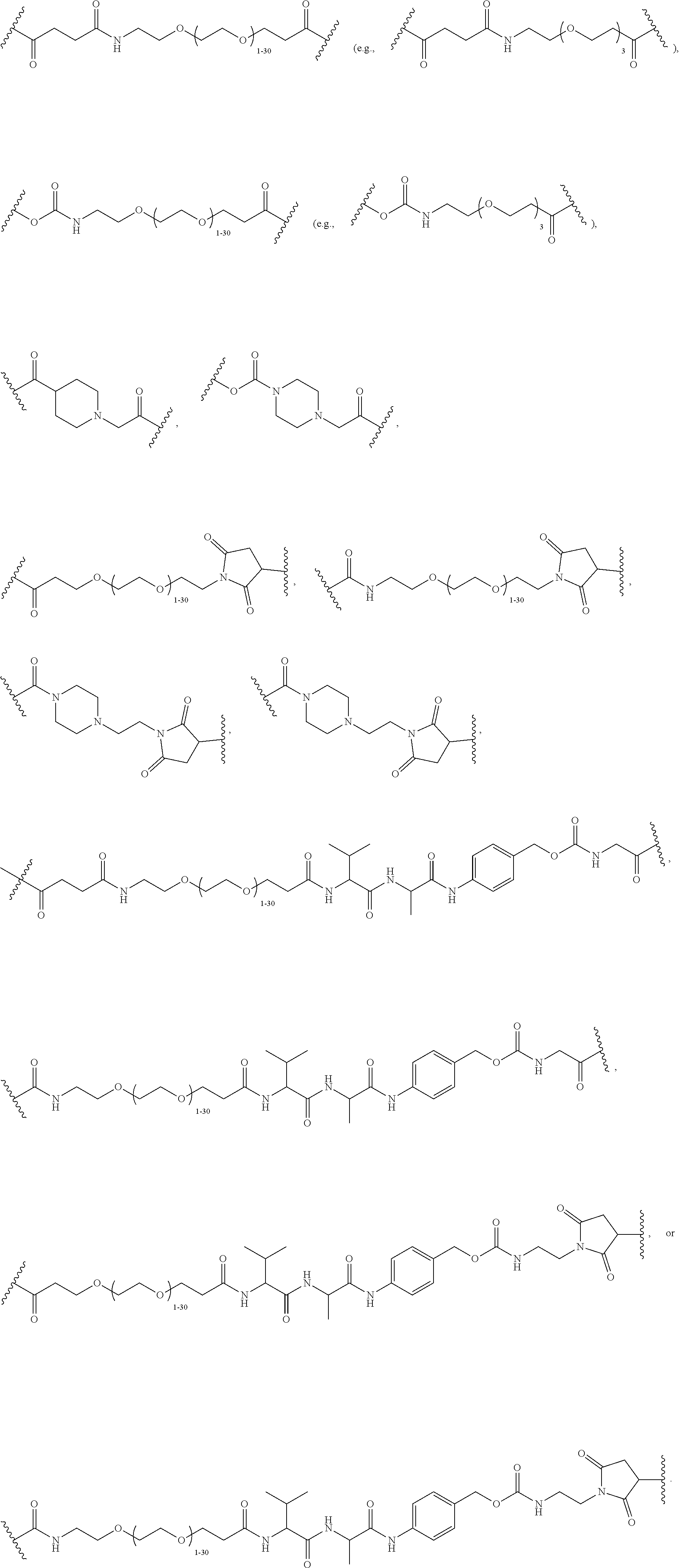

In some embodiments, the linker L comprises one or more of:

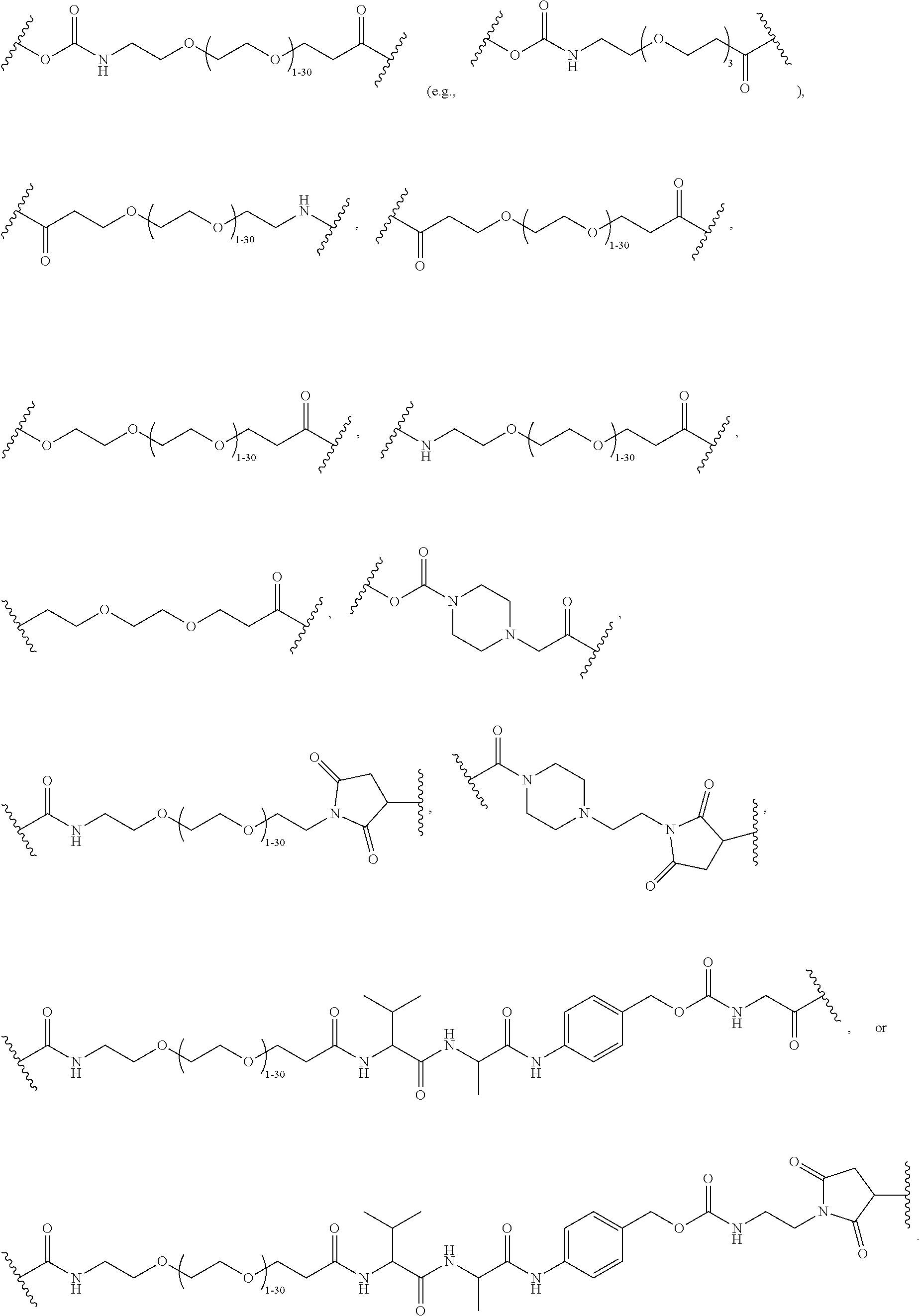

The foregoing linkers may bond to an amino acid side chain present on X, such as a lysine or cysteine (e.g.,

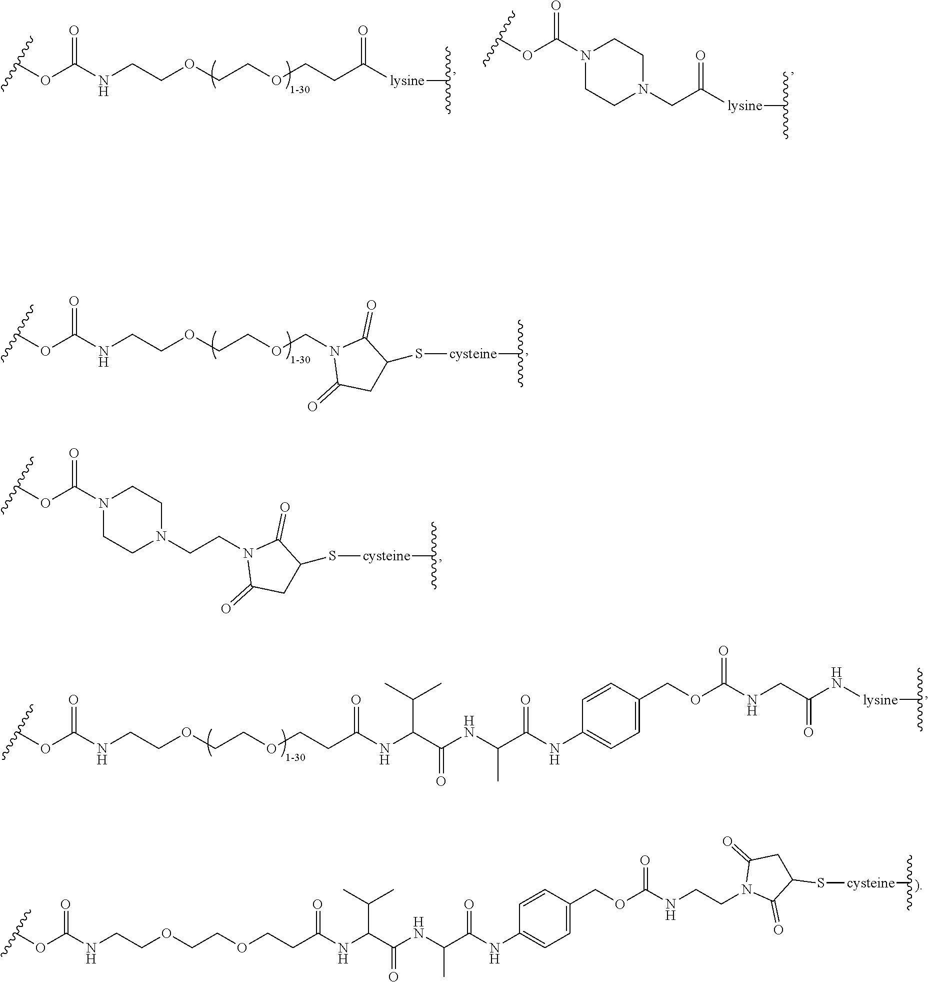

In some embodiments, the linker L is —C(O)L⁴- or —C(O)C₁₋₆alkyleneC(O)L⁴-;
L⁴ is a bond, —N(R¹²)—C₂₋₃alkylene-N(R¹³)C(O)—, —CH(NHC(O)R¹⁴)C₁₋₄alkylene-S—S—C₁₋₄alkylene-OC(O)—, —NHNHC(O)CH(NHC(O)R¹⁵)CH₂C(O)—, —C₁₋₆alkylene-CH(G^{x})OC(O)—,

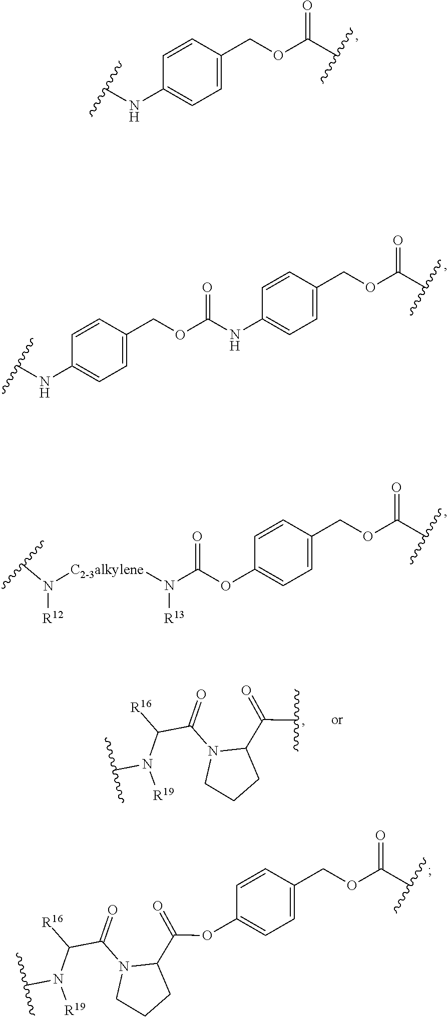

R¹², R¹³, R¹⁴, R¹⁵, and R¹⁹ are each independently hydrogen or C₁₋₄alkyl;
R¹⁶ is hydrogen, C₁₋₄alkyl, —C₁₋₄alkylene-OH, —C₁₋₄alkylene-OC₁₋₄alkyl, —C₁₋₄alkylene-CO₂H, or —C₁₋₄alkylene-CONH₂; and
G^{x} is phenyl optionally substituted with 1-5 substituents independently selected from the group consisting of halogen, C₁₋₄alkyl, C₁₋₄haloalkyl, C₁₋₄alkoxy, cyano, and nitro.

In some embodiments, the linker L comprises a carbonyl moiety for conjugating the tetrazine moiety to the linker or X. For example, the linker may comprise a polypeptide moiety (PPM) having the lysine residue and lysine side chain and the PPM may also have additional lysines, or other amino acid side chains conjugated to the carbonyl moiety. In some embodiments, the linker L may comprise

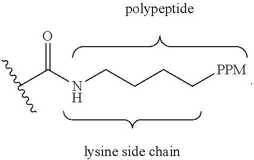

In some embodiments, the linker L is, or comprises one or more of:

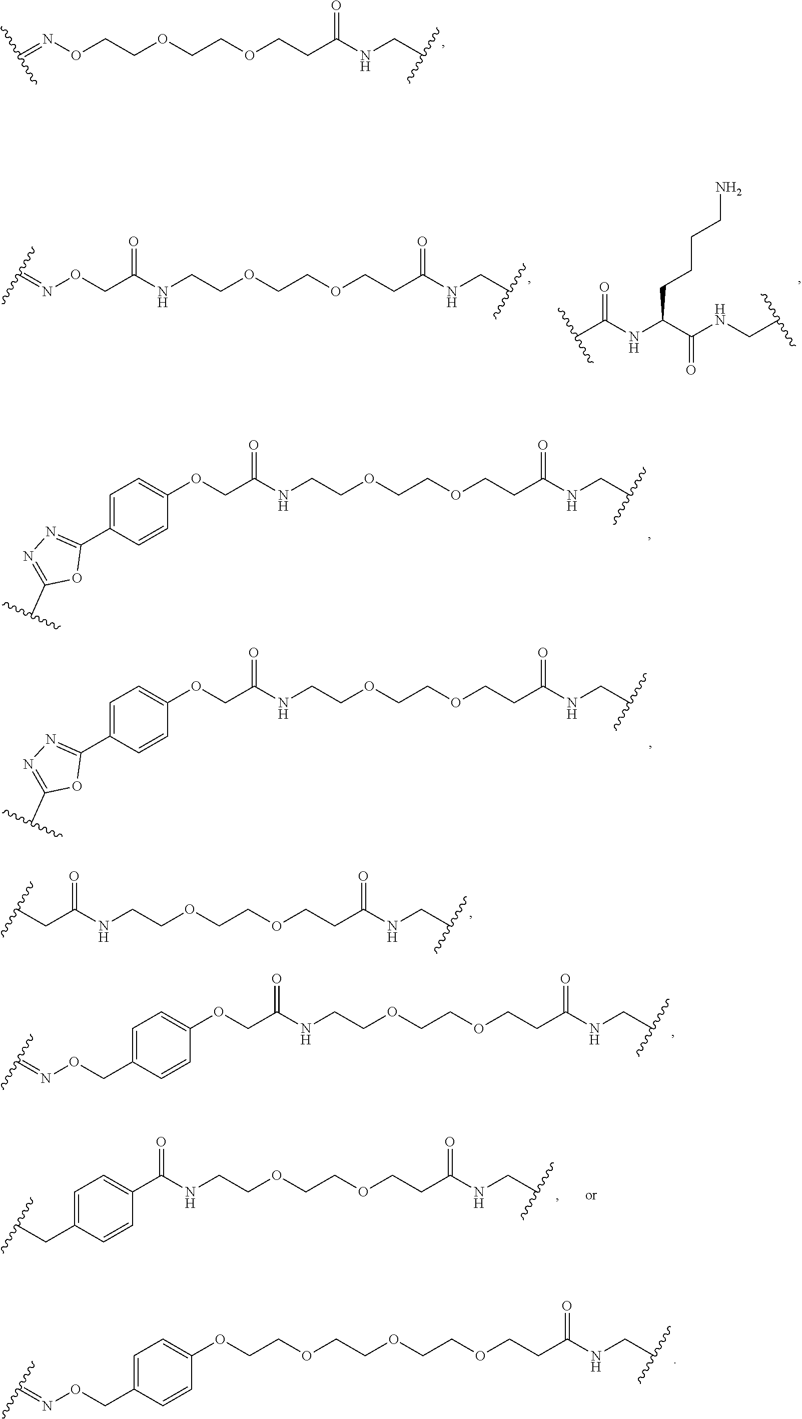

In some embodiments, the linker L is, or comprises one or more of:

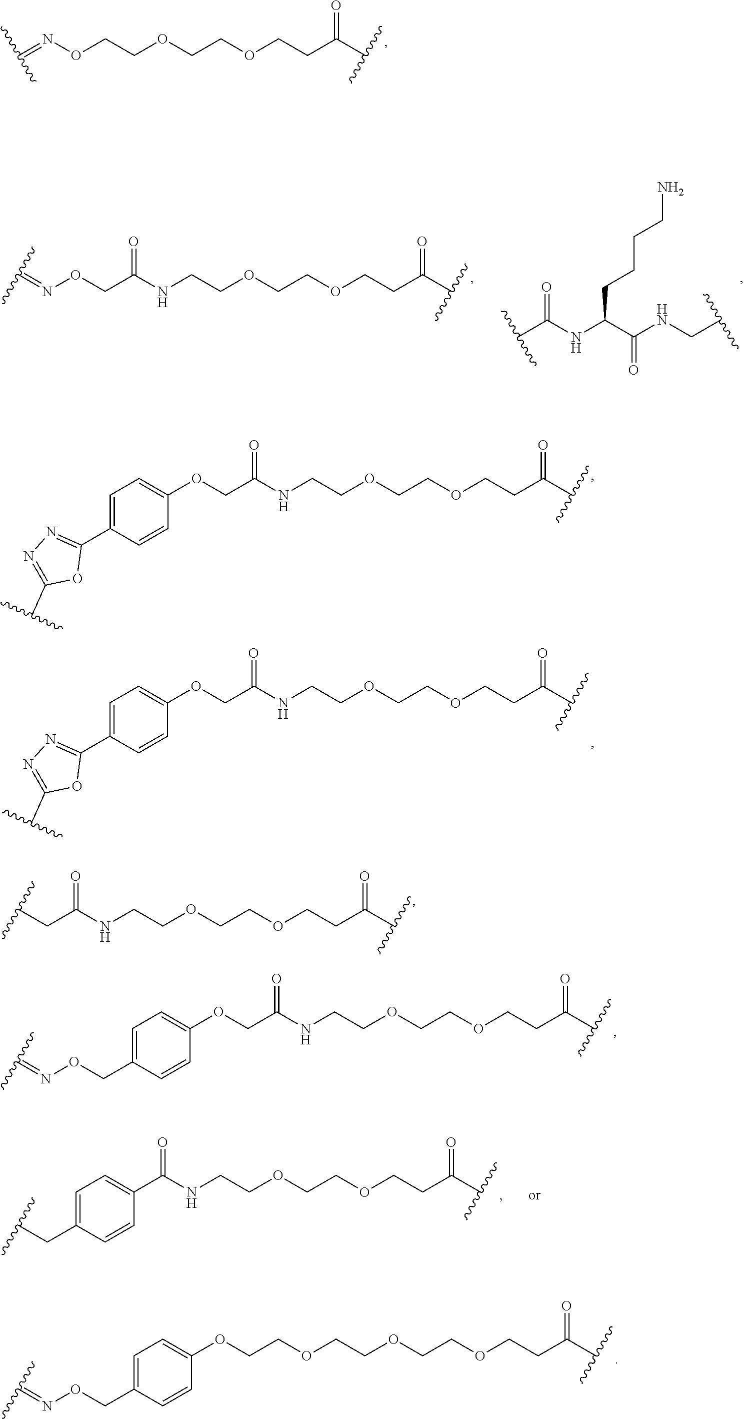

In some embodiments, the linker L is, or comprises one or more of:

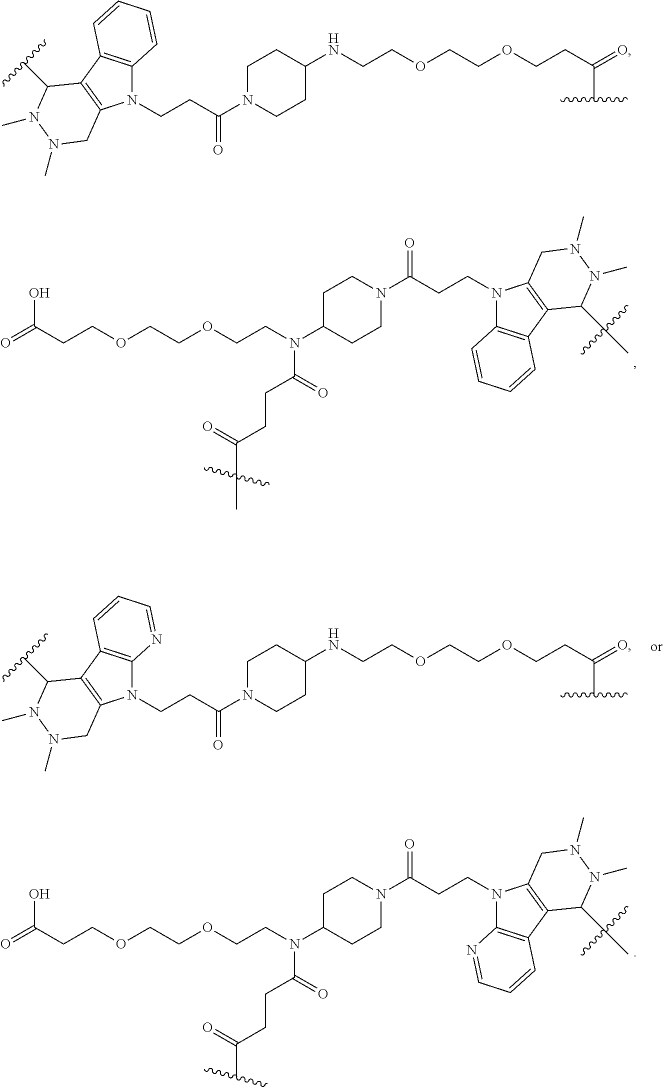

In some embodiments, the linker L is:

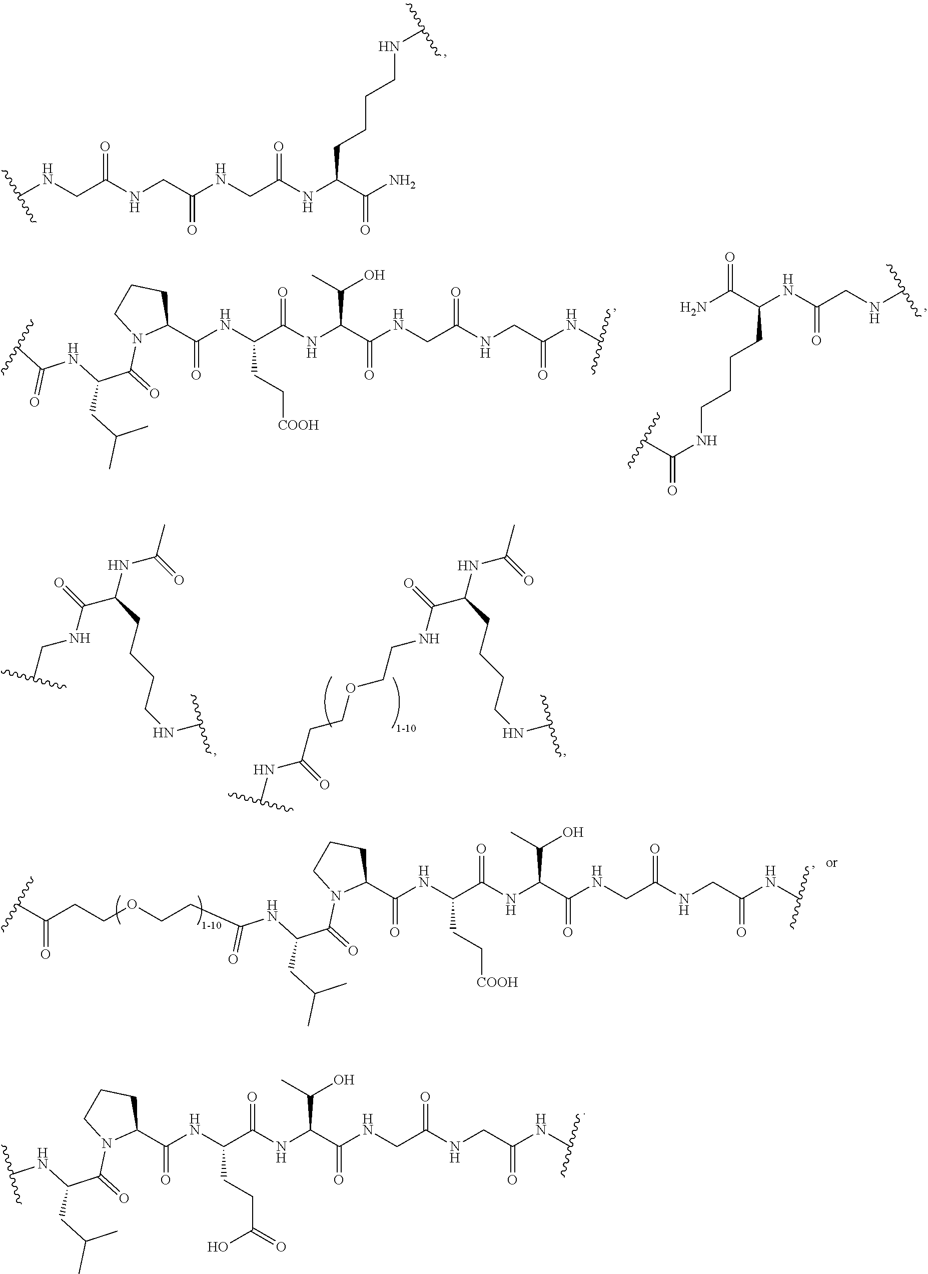

In some embodiments, the linker L is, or comprises one or more of

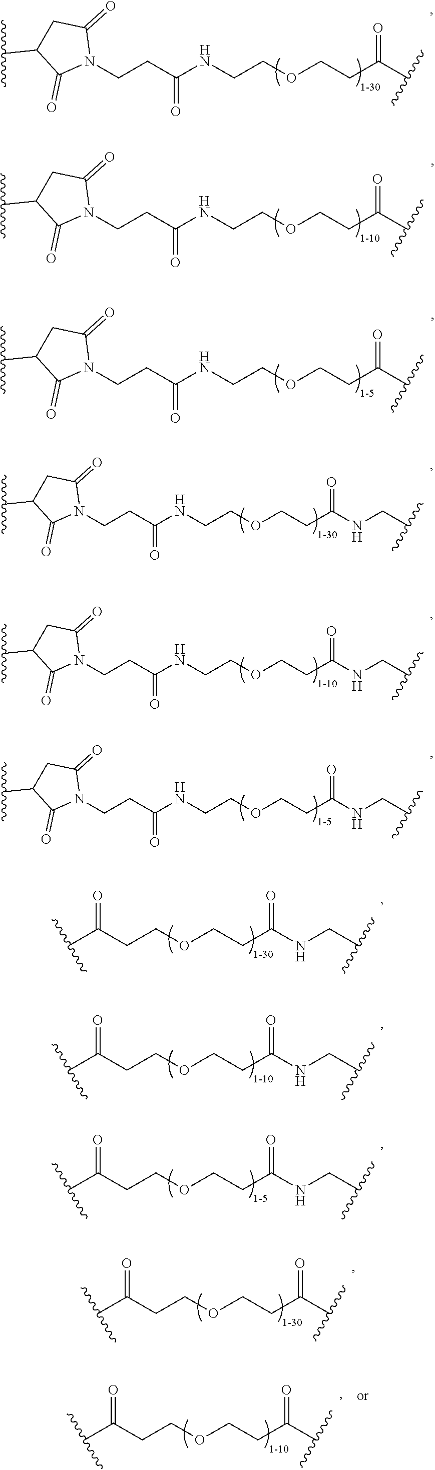

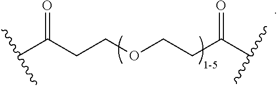

In some embodiments, ring A is pyrimidinyl, triazinyl, oxazolyl, isoxazole, imidazolyl, oxadiazolyl, 6,7-dihydro-5H-pyrrolo[3,4-d]pyrimidinyl, 5,6,7,8-tetrahydropyrido[4,3-d]pyrimidinyl, or 5,6,7,8-tetrahydropyrido[3,4-d]pyrimidinyl; wherein each may be optionally substituted.

In some embodiments, X is an antibody, or antibody fragment moiety, that targets HER2, TROP2, Nectin-4, FN1, FAP, TNC, or ECM.

In some embodiments, X is zolbetuximab, claudiximab, andecaliximab, anetumab, amatuximab, MMOT-0530A, L19, NJB2, F19, OMTX005, sibrotuzumab, F16, or R6N, trastuzumab, enfortumab, or sacituzumab, or an antibody fragment moiety derived therefrom.

In some embodiments, X is L19, NJB2, F19, OMTX005, sibrotuzumab, F16, or R6N, trastuzumab, enfortumab, or sacituzumab, or an antibody fragment moiety derived therefrom.

In some embodiments, p is 1-100. In some embodiments, p is 1-75. In some embodiments, p is 1-50. In some embodiments, p is 1-30. In some embodiments, p is 1-20. In some embodiments, p is 1-10. In some embodiments, p is 5-10. In some embodiments, p is 1-15. In some embodiments, p is 8-12.

In some embodiments, p is 1-12. In some embodiments, X is an antibody. In some embodiments, p is 1-6, or 5-6. In some embodiments, X is an antibody fragment moiety (e.g., Fab).

In some embodiments, p is 1-10, or 1-9, or 1-8, or 1-7, or 1-6, or 1-5, or 1-4, or 1-3, or 1-2, or 2-10, or 2-9, or 2-8, or 2-7, or 2-6, or 2-5, or 2-4, or 2-3, or 3-10, or 3-9, or 3-8, or 3-7, or 3-6, or 3-5, or 3-4, or 4-10, or 4-9, or 4-8, or 4-7, or 4-6, or 4-5, or 5-10, or 5-9, or 5-8, or 5-7, or 5-6, 6-10, or 6-9, or 6-8, or 6-7, 7-10, or 7-9, or 7-8, 8-10, or 8-9, or 9-10. In some embodiments, X is an antibody or an antibody fragment moiety.

In some embodiments, p is 1-16, or 1-8, or 1-7, or 1-6, or 1-5, or 1-4, or 1-3, or 1-2. In some embodiments, X is an antibody fragment moiety (e.g., Fab).

In certain embodiments, p is 1-10, or 1-9, or 1-8, or 1-7, or 1-6, or 1-5, or 1-4, or 1-3, or 1-2, or 2-10, or 2-9, or 2-8, or 2-7, or 2-6, or 2-5, or 2-4, or 2-3, or 3-10, or 3-9, or 3-8, or 3-7, or 3-6, or 3-5, or 3-4, or 4-10, or 4-9, or 4-8, or 4-7, or 4-6, or 4-5, or 5-10, or 5-9, or 5-8, or 5-7, or 5-6, 6-10, or 6-9, or 6-8, or 6-7, 7-10, or 7-9, or 7-8, 8-10, or 8-9, or 9-10, and X is an antibody or an antibody fragment moiety of from 15 KDa to 75 KDa, or 25-75 KDa, or 45-55 KDa, or less than 25 KDa, or less than 35 KDa, or less than 45 KDa, or about 50 KDa, or less than 55 KDa, or less than 65 KDa, or less than 75 KDa, or greater than 75 KDa.

In certain embodiments, p is dependent on the size and/or number of available binding sites on X for forming a covalent bond to L. In certain embodiments, when X is an antibody or an antibody fragment moiety greater than 75 KDa, p is 2-6. In certain embodiments, when X is an antibody or an antibody fragment moiety between 25-75 KDa, p is 1-4. In certain embodiments, when X is an antibody or an antibody fragment moiety between 45-55 KDa, p is 1-4. In certain embodiments, when X is an antibody or an antibody fragment moiety of less than 25 KDa, p is 1-3, or 2-3, or 1-2, or about 1, about 2, or about 3.

In certain embodiments, the tetrazine activator is a therapeutic support composition comprising a biocompatible support and a tetrazine-containing group.

In certain embodiments, the therapeutic support composition comprises a support. Supports may be biocompatible supports compositions, i.e., compatible with the subject's body. In some instances, a support is non-toxic to the subject and does not substantially react with tissue or biological compounds in the subject. For example, the support can be a hydrogel, among others. A support is capable of implantation into a subject's body and supporting binding agents (e.g., tetrazine-containing group). Representative supports include, but are not limited to polymers, viscous or non-viscous liquid materials, gels, hydrogels, polysaccharide hydrogels, a cross-linked polymer matrix, a metal, a ceramic, a plastic, a bone graft material, alginate, cellulose, chitosan, hyaluronic acid, chondroitin sulfate, heparin, and the like. Supports also include particles, such as nanoparticles, microparticles, and the like.

Hydrogels may be polysaccharide hydrogels, alginate, cellulose, hyaluronic acid, chitosan, chitosin, chitin, hyaluronic acid, chondroitin sulfate, heparin, and the like. Other suitable sugar-based biomaterials include those described in Polymer Advanced Technology, 2014, 25, 448-460. Polymers that may be used as the support can include, but are not limited to, polyphosphazenes, polyanhydrides, polyacetals, poly(ortho esters), polyphosphoesters, polycaprolactones, polyurethanes, polylactides, polycarbonates, polyamides, and polyethers, and blends/composites/co-polymers thereof. Representative polyethers include, but are not limited to, poly(ethylene glycol) (PEG), polypropylene glycol) (PPG), triblock Pluronic ([PEG]ₙ-[PPG]ₘ-[PEG]n), PEG diacrylate (PEGDA), and PEG dimethacrylate (PEGDMA). The support can also include proteins and other poly(amino acids), such as collagen, gelatin, elastin and elastin-like polypeptides, albumin, fibrin, poly(gamma-glutamic acid), poly(L-lysine), poly(L-glutamic acid), poly(aspartic acid), and the like.

In some embodiments, the support is a hydrogel. In some embodiments, the support is an alginate. In some embodiments, the support is chitin. In some embodiments, the support is a hyaluronic acid (e.g., a non-hydrogel hyaluronic acid substantially without crosslinks). In some embodiments, the support is chitosin.

In certain embodiments, the support is a particle. Particles of the present disclosure can have a diameter that is 2 cm or less, such as 1.5 cm or less, or 1 cm or less, or 0.5 cm or less. For example, the particles can be nanoparticles or microparticles. Nanoparticles include particles having average dimensions in the nanometer scale (e.g., 1000 nm or less). Microparticles are particles having average dimensions in the micrometer scale (e.g., 1000 μm or less). By “average” is meant the arithmetic mean. In some embodiments, the nanoparticles have a diameter ranging from 1 nm to 1 μm, such as from 10 nm to 1 μm, or 25 nm to 1 μm, or 50 nm to 1 μm, or 75 nm to 1 μm, or 100 nm to 1 μm, or 150 nm to 1 μm, or 200 nm to 1 μm, or 250 nm to 1 μm, or 300 nm to 1 μm, or 350 nm to 1 μm, or 400 nm to 1 μm, or 450 nm to 1 μm, or 500 nm to 1 μm. In other embodiments, the microparticles have a diameter ranging from 1 μm to 1 mm, such as from 10 μm to 1 mm, or 25 μm to 1 mm, or 50 μm to 1 mm, or 75 μm to 1 mm, or 100 μm to 1 mm, or 150 μm to 1 mm, or 200 μm to 1 mm, or 250 μm to 1 mm, or 300 μm to 1 mm, or 350 μm to 1 mm, or 400 μm to 1 mm, or 450 μm to 1 mm, or 500 μm to 1 mm. In further embodiments, small particles on the order of 10-100 nm in diameter may be assembled to form larger complexes, such as clusters or assemblies on the order of 1-10 m. Particles of the present disclosure may be substantially spherical, such that the particles have a substantially circular cross-section. Other particle shapes may also be used, such as, but not limited to, ellipsoid, cubic, cylindrical, conical, needle, or other irregular shapes.

A “particle” may take the form of any fabricated material, a molecule, cryptophan, a virus, a phage, etc. The particle may be composed of a material, such as, but not limited to, a metal, a ceramic, a plastic, a glass, a composite, a polymer, a hydrogel, and the like. For example, the particles may be made of an inert material, such as alginate or iron oxide. In some examples, the particles may be magnetic and can be formed from a paramagnetic, super-paramagnetic or ferromagnetic material, or other material that responds to a magnetic field. Further, a particle may be of any shape, for example, spheres, rods, non-symmetrical shapes, etc. The particles, or a group of several particles in a complex, may be functionalized with a receptor that has a specific affinity to bind to or interact with a clinically relevant substrate. The receptor may be inherent to the particle itself. For example, the particle itself may be a virus or a phage with an inherent affinity for certain substrates. Additionally or alternatively, the particles can be functionalized by covalently or otherwise attaching or associating a receptor that specifically binds or otherwise recognizes a particular clinically relevant substrate. The functionalized receptor can be an antibody, peptide, nucleic acid, phage, bacteria, virus, or any other molecule with a defined affinity for a target substrate. Examples of material that may be used for the “particles” and/or “carrier” include polylactic acid, polyglycolic acid, PLGA polymers, alginates and alginate derivatives, gelatin, collagen, fibrin, hyaluronic acid, laminin rich gels, agarose, natural and synthetic polysaccharides, polyamino acids, polypeptides, polyesters, poly anhydrides, polyphosphazines, poly(vinyl alcohols), poly(alkylene oxides), poly(allylamines)(PAM), poly(acrylates), modified styrene polymers, pluronic polyols, polyoxamers, poly(uronic acids), poly(vinylpyrrolidone) and copolymers or graft copolymers of any of the above. These examples do not limit their concentration, their cross-linking with different agents, their method of administration, their tailored degradation profiles and other characteristics known to those skilled in the art.

The particles, or a group of several particles in a complex, may be functionalized with a targeting agent (e.g., a ligand or antibody) that specifically binds (or substantially specifically binds) to a target (e.g., a target receptor or a cell surface target, such as a clinically relevant receptor or cell surface target (e.g., antigen)). The targeting agent may be attached directly to the particle itself. The targeting agent can be an antibody, peptide, nucleic acid, phage, bacteria, virus, or any other molecule with a specific affinity for a target receptor or cell surface target. In some instances, the receptor or cell surface target is PD-1, CTLA-4, HER2/neu, HER1/EGFR, VEGFR, 4-1BB, GITR, or other cellular receptors or cell surface targets.

In some embodiments, the targeting agent is a monoclonal antibody. A monoclonal antibody can be an entire monoclonal antibody, or a fragment thereof (e.g., antigen-binding fragment (Fab)). In certain embodiments, the targeting agent is an antibody, or antibody fragment, that targets one or more of CD25 (NCBI Gene ID 3559), CEA (NCBI Gene ID 634), CEACAM5 (NCBI Gene ID 1048), ASPH (NCBI Gene ID 444), EGFR (NCBI Gene ID 1956), EPCAM (NCBI Gene ID 4072), VEGFR (NCBI Gene ID 3791), PDGFR (NCBI Gene ID 5159), TROP2 (NCBI Gene ID 4070), Nectin4 (NCBI Gene ID 81607), PSMA (NCBI Gene ID 2346), BCMA (NCBI Gene ID 608), CD22 (NCBI Gene ID 933), CD20 (NCBI Gene ID 920), CD19 (NCBI Gene ID 930), CD79b (NCBI Gene ID 974), CD38 (NCBI Gene ID 952), CD45 (NCBI Gene ID 5788), Endoglin (NCBI Gene ID 2022), FGFR2 (NCBI Gene ID 14183), C4.4A (NCBI Gene ID 27076), Claudin-18.2 (NCBI Gene ID 51208), MMP9 (NCBI Gene ID 4318), Folate receptor (NCBI Gene ID 2348), DLL3 (NCBI Gene ID 10683), CD138 (NCBI Gene ID 6382), CD56 (NCBI Gene ID 4684), CD37 (NCBI Gene ID 951), CD74 (NCBI Gene ID 972), mesothelin (NCBI Gene ID 10232), IL-6R (NCBI Gene ID 3570), SLAMF7 (NCBI Gene ID 57823), BAFF (NCBI Gene ID 10673), MUC1 (NCBI Gene ID 4582), GPC3 (NCBI Gene ID 2719), HER2 (NCBI Gene ID 2064), HER3 (NCBI Gene ID 2065), CD30 (NCBI Gene ID 943), CD33 (NCBI Gene ID 945), CD123 (NCBI Gene ID 3563), GPNMB (NCBI Gene ID 10457), cMET (NCBI Gene ID 4233), CD142 (NCBI Gene ID 2152), NaPi2B (NCBI Gene ID 10568), GCC (NCBI Gene ID 2984), STEAP1 (NCBI Gene ID 26872), MUC16 (NCBI Gene ID 94025), CD70 (NCBI Gene ID 970), CD44 (NCBI Gene ID 960), (NCBI Gene ID), Antibody fragments (NCBI Gene ID), vWF (NCBI Gene ID 7450), TNF (NCBI Gene ID 7124), IL-6R (NCBI Gene ID 3570), BCMA (NCBI Gene ID 608), ADAMTS5 (NCBI Gene ID 11096), CX3CR1 (NCBI Gene ID 1524), CXCR4 (NCBI Gene ID 7852), TfR1 (NCBI Gene ID 7037), VEGFR (NCBI Gene ID 3791), or PSMA (NCBI Gene ID 2346).

In certain embodiments, the targeting agent is an antibody, or antibody fragment, that targets CD25, such as Daclizumab, RG6292, basiliximab, or HuMax-TAC, or an antibody fragment derived therefrom.

In certain embodiments, the targeting agent is an antibody, or antibody fragment, that targets CEA, such as Labetuzumab, 15-1-32, PR1A3, or cT84.66, or an antibody fragment derived therefrom.

In certain embodiments, the targeting agent is an antibody, or antibody fragment, that targets CEACAM5, such as Tusamitiamab or CC4, or an antibody fragment derived therefrom.

In certain embodiments, the targeting agent is an antibody, or antibody fragment, that targets ASPH, such as PAN-622, or an antibody fragment derived therefrom.

In certain embodiments, the targeting agent is an antibody, or antibody fragment, that targets EGFR, such as Cetuximab, necitumumab, nimotuzumab, matuzumab, AMG595, Depatuxizumab, Dapatuxizumab, duligotuzumab, Futuximab, GC1118, Imgatuzumab, Panitumumab, Alutumumab, Tomuzotuximab, or Laprituximab, or an antibody fragment derived therefrom.

In certain embodiments, the targeting agent is an antibody, or antibody fragment, that targets EPCAM, such as Oportuzumab, Citatuzumab, Tucotuzumab, Catumaxomab, Edrecolomab, or Adecatumumab, or an antibody fragment derived therefrom.

In certain embodiments, the targeting agent is an antibody, or antibody fragment, that targets VEGFR, such as Ramucizumab, Ramucirumab, or Vulinacimab, or an antibody fragment derived therefrom.

In certain embodiments, the targeting agent is an antibody, or antibody fragment, that targets PDGFR, such as Olaratumab or Ramucirumab, or an antibody fragment derived therefrom.

In certain embodiments, the targeting agent is an antibody, or antibody fragment, that targets TROP2, such as Sacituzumab or Pr1E11, or an antibody fragment derived therefrom.

In certain embodiments, the targeting agent is an antibody, or antibody fragment, that targets Nectin4, such as Enfortumab, or an antibody fragment derived therefrom.

In certain embodiments, the targeting agent is an antibody, or antibody fragment, that targets PSMA, such as J591 or MLN591, or an antibody fragment derived therefrom.

In certain embodiments, the targeting agent is an antibody, or antibody fragment, that targets BCMA, such as Belantamab, or an antibody fragment derived therefrom.

In certain embodiments, the targeting agent is an antibody, or antibody fragment, that targets CD22, such as Moxetumomab, Inotuzumab, Epratuzumab, or Pinatuzumab, or an antibody fragment derived therefrom.

In certain embodiments, the targeting agent is an antibody, or antibody fragment, that targets CD20, such as Ublituximab, Ofatumumab, Rituximab, Obinutuzumab, tositumomab, or ibritumomab, or an antibody fragment derived therefrom.

In certain embodiments, the targeting agent is an antibody, or antibody fragment, that targets CD19, such as Loncastuximab, XMAB-5574, MOR208, Coltuximab, Denintuzumab, taplitumomab, or MDX-1342, or an antibody fragment derived therefrom.

In certain embodiments, the targeting agent is an antibody, or antibody fragment, that targets CD79b, such as Polatuzumab, or an antibody fragment derived therefrom.

In certain embodiments, the targeting agent is an antibody, or antibody fragment, that targets CD38, such as Isatuximab, daratumumab, MOR202, or TAK-079, or an antibody fragment derived therefrom.

In certain embodiments, the targeting agent is an antibody, or antibody fragment, that targets CD45, such as I-131-BC8, or Iomab-B, or an antibody fragment derived therefrom.

In certain embodiments, the targeting agent is an antibody, or antibody fragment, that targets Endoglin, such as Carotuximab, or an antibody fragment derived therefrom.

In certain embodiments, the targeting agent is an antibody, or antibody fragment, that targets FGFR2, such as Bemarituzumab or Aprutumab, or an antibody fragment derived therefrom.

In certain embodiments, the targeting agent is an antibody, or antibody fragment, that targets C4.4A, such as Lupartumab, or an antibody fragment derived therefrom.

In certain embodiments, the targeting agent is an antibody, or antibody fragment, that targets Claudin-18.2, such as Zolbetuximab, or claudiximab, or an antibody fragment derived therefrom.

In certain embodiments, the targeting agent is an antibody, or antibody fragment, that targets MMP9, such as Andecaliximab, or an antibody fragment derived therefrom.

In certain embodiments, the targeting agent is an antibody, or antibody fragment, that targets Folate receptor, such as Mirvetuximab, Farletuzumab, MORAb-202, MORAb-003, or SP8166, or an antibody fragment derived therefrom.

In certain embodiments, the targeting agent is an antibody, or antibody fragment, that targets DLL3, such as Rovalpituzumab, or an antibody fragment derived therefrom.

In certain embodiments, the targeting agent is an antibody, or antibody fragment, that targets CD138, such as Indatuximab, or an antibody fragment derived therefrom.

In certain embodiments, the targeting agent is an antibody, or antibody fragment, that targets CD56, such as Lorvotuzumab, Promiximab, or an antibody fragment derived therefrom.

In certain embodiments, the targeting agent is an antibody, or antibody fragment, that targets CD37, such as BI 836826, Otlertuzumab, or Naratuximab, or an antibody fragment derived therefrom.

In certain embodiments, the targeting agent is an antibody, or antibody fragment, that targets CD74, such as Milatuzumab, or an antibody fragment derived therefrom.

In certain embodiments, the targeting agent is an antibody, or antibody fragment, that targets mesothelin, such as Anetumab, Amatuximab, or MMOT-0530A, or an antibody fragment derived therefrom.

In certain embodiments, the targeting agent is an antibody, or antibody fragment, that targets IL-6R, such as Tocilizumab or Sarilumab, or an antibody fragment derived therefrom.

In certain embodiments, the targeting agent is an antibody, or antibody fragment, that targets SLAMF7, such as Elotuzumab, or an antibody fragment derived therefrom.

In certain embodiments, the targeting agent is an antibody, or antibody fragment, that targets BAFF, such as Belimumab, or an antibody fragment therefrom.

In certain embodiments, the targeting agent is an antibody, or antibody fragment, that targets MUC1, such as KL-6, MY.1E12, hMUC1-1H7, TAB004, huC242, Clivatuzumab, 8HuDS6, Gatipotuzumab, AR20.5, or Cantuzumab, or an antibody fragment derived therefrom.

In certain embodiments, the targeting agent is an antibody, or antibody fragment, that targets GPC3, such as Codrituzumab, ECT204, or MDX-1414, or an antibody fragment derived therefrom.

In certain embodiments, the targeting agent is an antibody, or antibody fragment, that targets HER2, such as Pertuzumab, trastuzumab, or Margetuximab, or an antibody fragment derived therefrom.

In certain embodiments, the targeting agent is an antibody, or antibody fragment, that targets HER3, such as Patritumab, Seribantumab, Lumretuzumab, Elgemtumab, AV-203, CDX-3379, or GSK284933, or an antibody fragment derived therefrom.

In certain embodiments, the targeting agent is an antibody, or antibody fragment, that targets CD30, such as Brentuximab, or an antibody fragment derived therefrom.

In certain embodiments, the targeting agent is an antibody, or antibody fragment, that targets CD33, such as Gemtuzumab, BI 835858, vadastuximab, or lintuzumab, or an antibody fragment derived therefrom.

In certain embodiments, the targeting agent is an antibody, or antibody fragment, that targets CD123, such as KHK2823, Taclotuzumab, or G4723A, or an antibody fragment derived therefrom.

In certain embodiments, the targeting agent is an antibody, or antibody fragment, that targets GPNMB, such as Glembatumumab, or an antibody fragment derived therefrom.

In certain embodiments, the targeting agent is an antibody, or antibody fragment, that targets cMET, such as Telisotuzumab, Onartuzumab, or SAIT301, or an antibody fragment derived therefrom.

In certain embodiments, the targeting agent is an antibody, or antibody fragment, that targets CD142, such as Tisotumab, or an antibody fragment derived therefrom.

In certain embodiments, the targeting agent is an antibody, or antibody fragment, that targets NaPi2B, such as Lifastuzumab, or an antibody fragment derived therefrom.

In certain embodiments, the targeting agent is an antibody, or antibody fragment, that targets GCC, such as Indusatumab, or an antibody fragment derived therefrom.

In certain embodiments, the targeting agent is an antibody, or antibody fragment, that targets STEAP1, such as Vandortuzumab, or an antibody fragment derived therefrom.

In certain embodiments, the targeting agent is an antibody, or antibody fragment, that targets MUC16, such as Sofituzumab, or an antibody fragment derived therefrom.

In certain embodiments, the targeting agent is an antibody, or antibody fragment, that targets CD70, such as Vorsetuzumab, or an antibody fragment derived therefrom.

In certain embodiments, the targeting agent is an antibody, or antibody fragment, that targets CD44, such as Bivatuzumab, or an antibody fragment derived therefrom.

In certain embodiments, the targeting agent is an antibody, or antibody fragment, that targets vWF, such as Caplacizumab, or an antibody fragment derived therefrom.

In certain embodiments, the targeting agent is an antibody, or antibody fragment, that targets TNF, such as Ozoralizumab, V565, or PF-05230905, or an antibody fragment derived therefrom.

In certain embodiments, the targeting agent is an antibody, or antibody fragment, that targets IL-6R, such as Vobarilizumab, or an antibody fragment derived therefrom.

In certain embodiments, the targeting agent is an antibody, or antibody fragment, that targets BCMA, such as LCAR-B38M, or an antibody fragment derived therefrom.

In certain embodiments, the targeting agent is an antibody, or antibody fragment, that targets ADAMTS5, such as M6495, or an antibody fragment derived therefrom.

In certain embodiments, the targeting agent is an antibody, or antibody fragment, that targets CX3CR1, such as BI 655088, or an antibody fragment derived therefrom.

In certain embodiments, the targeting agent is an antibody, or antibody fragment, that targets CXCR4, such as AD-214 or ALX-0651, or an antibody fragment derived therefrom.

In certain embodiments, the targeting agent is an antibody, or antibody fragment, that targets TfR1, such as TXB4, or an antibody fragment derived therefrom.

In certain embodiments, the targeting agent is an antibody, or antibody fragment, that targets VEGFR, such as CDP791, or an antibody fragment derived therefrom.

In certain embodiments, the targeting agent is an antibody, or antibody fragment, that targets PSMA, such as GY1, or an antibody fragment derived therefrom.

Other compounds or molecules, such as fluorophores or autofluorescent or luminescent markers, which may assist in detecting the particles (e.g., in vivo detection), may also be attached to the particles. The ligands and/or detectable labels may be attached directly to the particle or attached to the particle through bioorthogonal functional groups as described herein.

In certain embodiments, the support is a bone graft material, such as a bone graft substitute material. A bone graft substitute material is a material structurally similar to bone. In some instances, a bone graft substitute material is bioresorbable such that the bone graft substitute material can dissolve or be absorbed in the body over time. A bone graft substitute material can be osteoconductive, such that it facilitates blood vessel and new bone formation into the bone graft substitute material. In some instances, the bone graft substitute material is osteoinductive, such that it facilitates the formation of new bone through active recruitment of mesenchymal stem cells from the surrounding tissue. For example, growth factors, such as bone morphogenetic proteins, may be included in the bone graft substitute material. Bone graft substitute materials include, but are not limited to, hydroxyapatite, tricalcium phosphate, demineralized bone matrix, bovine collagen, calcium sulfate, calcium phosphate, cancellous bone chips, and the like, and combinations thereof.

Therapeutic support compositions of the present disclosure include a support and a first binding agent covalently linked to the support. The binding agent may be attached to the support on a surface of the support, such as a solvent-accessible surface of the support (e.g., a surface of the support that is in contact with the surrounding solvent). In some cases, the binding agent is attached directly to the support. For example, the binding agent may be covalently attached to the surface of the support, e.g., through a covalent bond, such as an amide, amine, ester, carbamate, urea, thioether, thiocarbamate, thiocarbonate, thiourea, etc. In some instances, the binding agent is covalently attached to the support through an amide bond. In other instances, the binding agent may be linked to the support via a linker. Any suitable linker can be used to link the binding agent to the support. Representative linkers can have from 1 to 100 linking atoms, and can include ethylene-oxy groups, amines, esters, amides, carbamates, carbonates, and ketone functional groups. For example, linkers may have from 1 to 50 linking atoms, or from 5 to 50 linking atoms, or from 10 to 50 linking atoms. Representative linkers include, but are not limited to, those shown below:

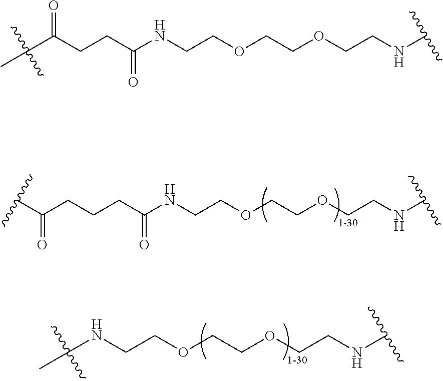

In certain embodiments, the therapeutic support compositions comprise a support and a tetrazine-containing group of Formula IVA, IVB, or IVC:

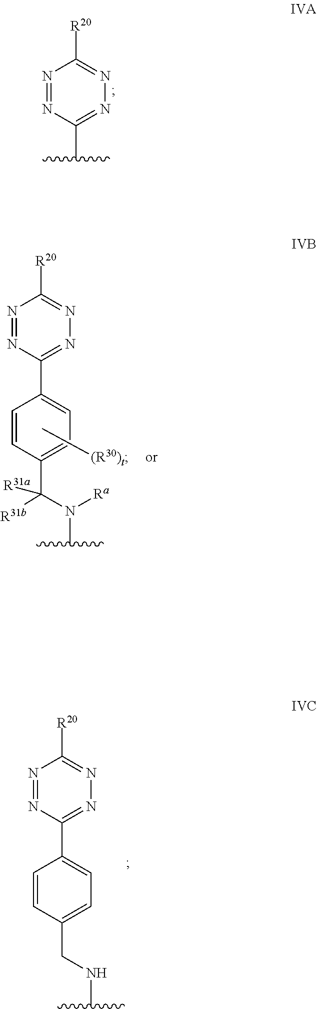

wherein R²⁰ is selected from the group consisting of hydrogen, halogen, cyano, nitro, alkyl, alkenyl, alkynyl, heteroalkyl, aryl, heteroaryl, heterocycle, cycloalkyl, cycloalkenyl, CF₃, CF₂—R′, NO₂, OR′, SR′, C(═O)R′, C(═S)R′, OC(═O)R′″, SC(═O)R″, OC(═S)R′″, SC(═S)R′″, S(═O)R′, S(═O)₂R′″, S(═O)₂NR′R″, C(═O)O—R′, C(═O)S—R′, C(═S)O—R′, C(═S)S—R′, C(═O)NR′R″, C(═S)NR′R″, NR′R″, NR′C(═O)R″, NR′C(═S)R″, NR′C(═O)OR″, NR′C(═S)OR″, NR′C(═O)SR″, NR′C(═S)SR″, OC(═O)NR′R″, SC(═O)NR′R″, OC(═S) R′R′″, SC(═S)R′R″, NR′C(═O)NR″R″, and NR′C(═S)NR″R″; R′ and R″ at each occurrence are independently selected from hydrogen, aryl and alkyl; and R′″ at each occurrence is independently selected from aryl and alkyl; R³¹ is halogen, cyano, nitro, hydroxy, alkyl, haloalkyl; alkenyl, alkynyl, alkoxy; haloalkoxy; heteroalkyl, aryl, heteroaryl, heterocycle, cycloalkyl, or cycloalkenyl; R^{a}, R^{31a} and R^{31b} are each independently hydrogen, C₁-C₆-alkyl, or C₁-C₆-haloalkyl; and t is 0, 1, 2, 3, or 4.

In certain embodiments, the therapeutic support compositions have formula:

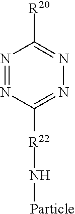

wherein
R²⁰ is selected from the group consisting of hydrogen, halogen, cyano, nitro, alkyl, alkenyl, alkynyl, heteroalkyl, aryl, heteroaryl, heterocycle, cycloalkyl, cycloalkenyl, CF₃, CF₂—R′, NO₂, OR′, SR′, C(═O)R′, C(═S)R′, OC(═O)R′″, SC(═O)R″, OC(═S)R′″, SC(═S)R′″, S(═O)R′, S(═O)₂R′″, S(═O)₂NR′R″, C(═O)O—R′, C(═O)S—R′, C(═S)O—R′, C(═S)S—R′, C(═O)NR′R″, C(═S)NR′R″, NR′R″, NR′C(═O)R″, NR′C(═S)R″, NR′C(═O)OR″, NR′C(═S)OR″, NR′C(═O)SR″, NR′C(═S)SR″, OC(═O)NR′R″, SC(═O)NR′R″, OC(═S) R′R′″, SC(═S)R′R″, NR′C(═O)NR″R″, and NR′C(═S)NR″R″; R′ and R″ at each occurrence are independently selected from hydrogen, aryl and alkyl; R′″ at each occurrence is independently selected from aryl and alkyl; and R²² is a linker of 1 to 100 linking atoms, and can include ethylene-oxy groups, amines, esters, amides, carbamates, carbonates, and ketone functional groups. For example, linkers may have from 1 to 50 linking atoms, or from 5 to 50 linking atoms, or from 10 to 50 linking atoms.

In certain embodiments, the therapeutic support compositions have formula:

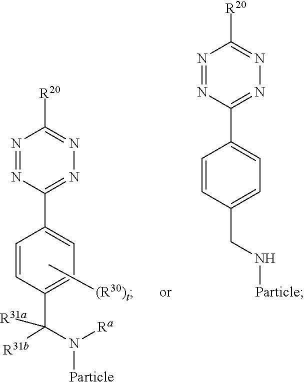

wherein
R²⁰ is selected from the group consisting of hydrogen, halogen, cyano, nitro, alkyl, alkenyl, alkynyl, heteroalkyl, aryl, heteroaryl, heterocycle, cycloalkyl, cycloalkenyl, CF₃, CF₂—R′, NO₂, OR′, SR′, C(═O)R′, C(═S)R′, OC(═O)R′″, SC(═O)R″, OC(═S)R′″, SC(═S)R′″, S(═O)R′, S(═O)₂R′″, S(═O)₂NR′R″, C(═O)O—R′, C(═O)S—R′, C(═S)O—R′, C(═S)S—R′, C(═O)NR′R″, C(═S)NR′R″, NR′R″, NR′C(═O)R″, NR′C(═S)R″, NR′C(═O)OR″, NR′C(═S)OR″, NR′C(═O)SR″, NR′C(═S)SR″, OC(═O)NR′R″, SC(═O)NR′R″, OC(═S) R′R′″, SC(═S)R′R″, NR′C(═O)NR″R″, and NR′C(═S)NR″R″; R′ and R″ at each occurrence are independently selected from hydrogen, aryl and alkyl; R′″ at each occurrence is independently selected from aryl and alkyl; R³⁰ is halogen, cyano, nitro, hydroxy, alkyl, haloalkyl; alkenyl, alkynyl, alkoxy; haloalkoxy; heteroalkyl, aryl, heteroaryl, heterocycle, cycloalkyl, or cycloalkenyl; R^{a}, R^{31a} and R^{31b} are each independently hydrogen, C1-C₆-alkyl, or C1-C₆-haloalkyl; and t is 0, 1, 2, 3, or 4.

In certain embodiments, the therapeutic support compositions comprise substituted alginate having units of formula:

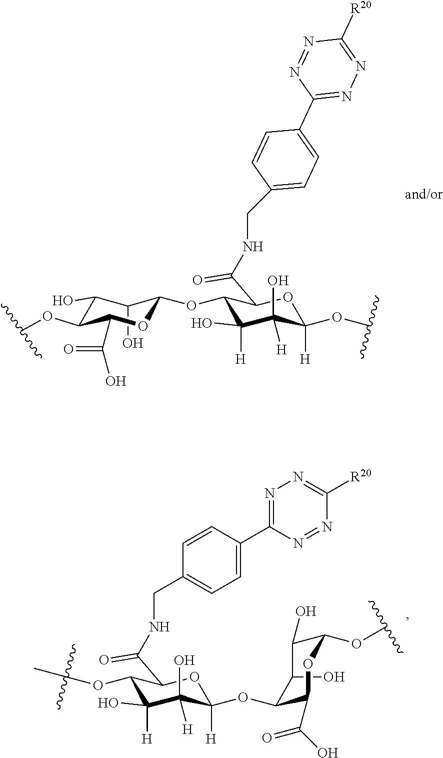

or a salt thereof,
wherein R²⁰ is selected from the group consisting of hydrogen, halogen, cyano, nitro, alkyl, alkenyl, alkynyl, heteroalkyl, aryl, heteroaryl, heterocycle, cycloalkyl, cycloalkenyl, CF₃, CF₂—R′, NO₂, OR′, SR′, C(═O)R′, C(═S)R′, OC(═O)R′″, SC(═O)R″, OC(═S)R′″, SC(═S)R′″, S(═O)R′, S(═O)₂R′″, S(═O)₂NR′R″, C(═O)O—R′, C(═O)S—R′, C(═S)O—R′, C(═S)S—R′, C(═O)NR′R″, C(═S)NR′R″, NR′R″, NR′C(═O)R″, NR′C(═S)R″, NR′C(═O)OR″, NR′C(═S)OR″, NR′C(═O)SR″, NR′C(═S)SR″, OC(═O)NR′R″, SC(═O)NR′R″, OC(═S) R′R′″, SC(═S)R′R″, NR′C(═O)NR″R″, and NR′C(═S)NR″R″; R′ and R″ at each occurrence are independently selected from hydrogen, aryl and alkyl; and R′″ at each occurrence is independently selected from aryl and alkyl.

In certain embodiments, the therapeutic support composition comprises units of formula:

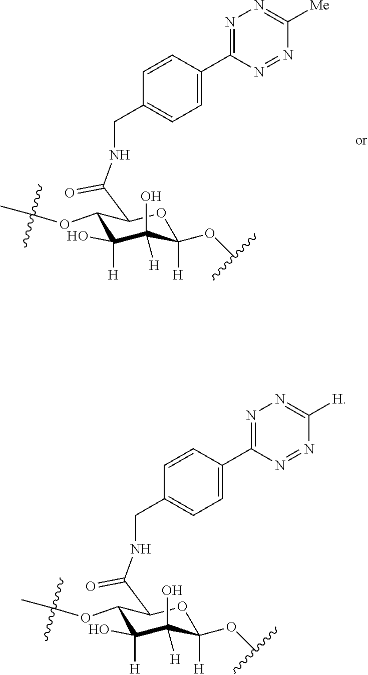

In some embodiments, the therapeutic support compositions comprise units of formula:

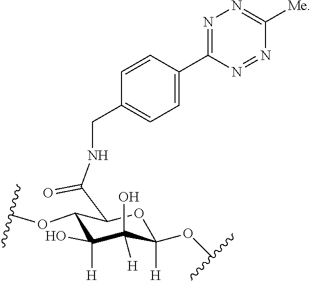

In some embodiments, the therapeutic support compositions comprise units of formula:

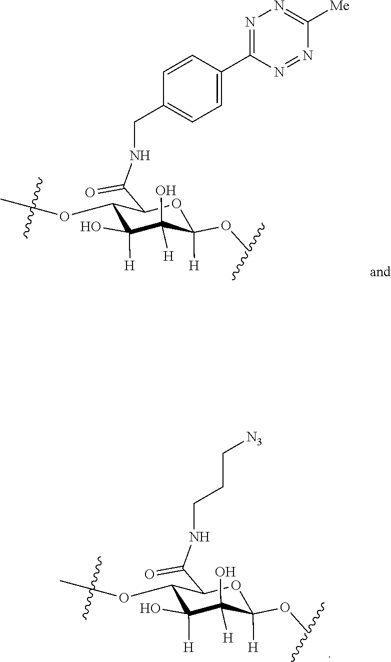

In some embodiments, the therapeutic support compositions comprise substituted hyaluronic acid having units of formula (II):

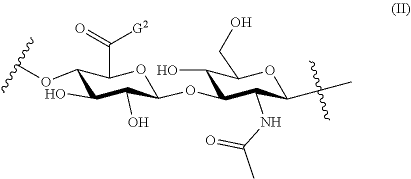

wherein G² is

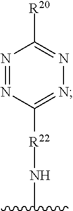

R²² is a linker of 1 to 100 linking atoms; and R²⁰ is as defined herein.

In further embodiments, G² is

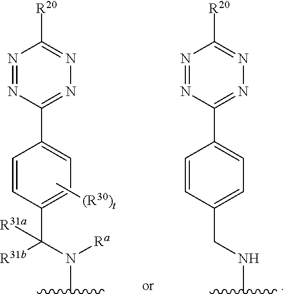

In still further embodiments, G² is

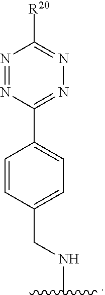

and R²⁰ is hydrogen or C₁₋₄alkyl.

Compounds of formula (II) include compounds of formula (II-A):

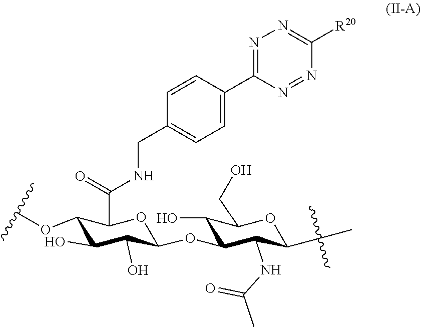

wherein R²⁰ is selected from the group consisting of hydrogen, halogen, cyano, nitro, alkyl, alkenyl, alkynyl, heteroalkyl, aryl, heteroaryl, heterocycle, cycloalkyl, cycloalkenyl, CF₃, CF₂—R′, NO₂, OR′, SR′, C(═O)R′, C(═S)R′, OC(═O)R′″, SC(═O)R″, OC(═S)R′″, SC(═S)R′″, S(═O)R′, S(═O)₂R′″, S(═O)₂NR′R″, C(═O)O—R′, C(═O)S—R′, C(═S)O—R′, C(═S)S—R′, C(═O)NR′R″, C(═S)NR′R″, NR′R″, NR′C(═O)R″, NR′C(═S)R″, NR′C(═O)OR″, NR′C(═S)OR″, NR′C(═O)SR″, NR′C(═S)SR″, OC(═O)NR′R″, SC(═O)NR′R″, OC(═S) R′R′″, SC(═S)R′R″, NR′C(═O)NR″R″, and NR′C(═S)NR″R″; R′ and R″ at each occurrence are independently selected from hydrogen, aryl and alkyl; and R′″ at each occurrence is independently selected from aryl and alkyl. In further embodiments according to formula (II-A), R²⁰ is hydrogen or C₁₋₄alkyl.

In some embodiments, the therapeutic support compositions comprise units of formula:

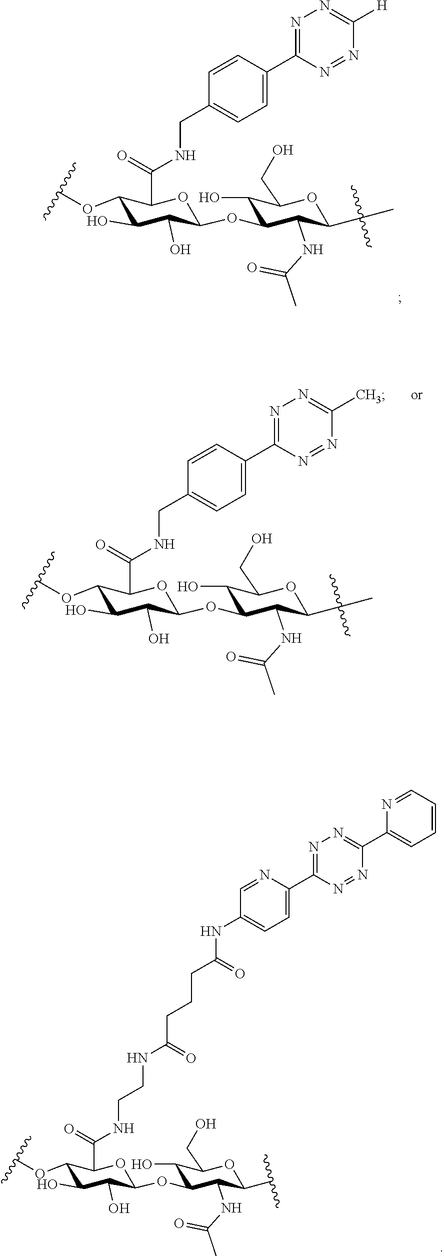

Additional therapeutic support compositions are exemplified in WO2017/044983, WO/2015/139025A1, and WO/2014/205126A1, the entire contents of each of which is incorporated herein by reference in their entirety.

The hyaluronic acid derivative includes a hyaluronic acid having a plurality of glucuronic acid units and a tetrazine-containing group linked or directly bonded to a glucuronic acid unit of the hyaluronic acid. The hyaluronic acid may also have a plurality of N-acetylglucosamine units. In certain embodiments, the N-acetylglucosamine units of the hyaluronic acid are not linked or conjugated to the tetrazine-containing group.

The tetrazine-containing group can be linked or directly bonded through a carboxylic acid of a glucuronic acid unit. The tetrazine-containing group can be incorporated into the hyaluronic acid from about 0.1% to about 80% as measured by the % of carboxylic acids being linked or conjugated to the tetrazine-containing group, such as about 1% to about 75%, about 5% to about 75%, about 10% to about 50%, or about 40% to about 75% as measured by the % of carboxylic acids being linked or conjugated to the tetrazine-containing group.

### D. Methods of Treatment

Aspects of the present disclosure include methods for delivering a bispecific antibody to a target location in a subject. The bispecific antibodies disclosed herein comprise a first antigen target binding moiety which is capable of targeting a specific antigen at a treatment site or on a cell in a subject. Thus, selective delivery can be achieved to a target location (e.g., an organ or tissue, or portion thereof), without targeting other locations in the subject (e.g., other organs or tissues, or portions thereof) that do not need treatment. The target location or treatment site can be a stationary location (e.g., an organ or tissue, tumor, or portion thereof) or a systemic location (e.g., cells in the blood or other bodily fluid).

The bispecific antibody is designed to target a treatment site in a subject via a first antigen target binding moiety, and upon activation (e.g., contact with a tetrazine activator), expose a second antigen target binding moiety which is capable of binding antigens on therapeutic antibodies or cells present in the subject.

In certain embodiments, the method comprises administering a therapeutically effective amount of the trans-cyclooctene-modified bispecific antibody to a subject in need thereof, followed by administering a tetrazine activator. In certain embodiments, the trans-cyclooctene-modified bispecific antibody is administered systemically. In certain embodiments, the tetrazine activator is administered systemically. In certain embodiments, the trans-cyclooctene-modified bispecific antibody is administered locally. In certain embodiments, the tetrazine activator is administered locally. In certain embodiments, both the trans-cyclooctene-modified bispecific antibody and the tetrazine activator are administered systemically. In certain embodiments, the tetrazine activator is administered locally and the trans-cyclooctene-modified bispecific antibody is administered systemically.

In certain embodiments, the method comprises administering the tetrazine activator locally as a therapeutic support composition, and then systemically administering the trans-cyclooctene-modified bispecific antibody. Due to the localized administration to a desired location in the subject, the bispecific antibody, the bispecific antibody will localize the second antigen target binding moiety at the desired target location.

In certain embodiments, where the tetrazine activator comprises a support composition, the method includes administering to the subject a trans-cyclooctene-modified bispecific antibody such that a bioorthogonal reaction between the tetrazine and trans-cyclooctene-modified bispecific antibody results in the bispecific antibody being bound to the support composition to form a support complex. In some embodiments, when the bioorthogonal reaction between the tetrazine and trans-cyclooctene-modified bispecific antibody occurs, the bispecific antibody may be released from the support complex. For example, the trans-cyclooctene-modified bispecific antibody may be administered systemically to the subject. Upon administration of the trans-cyclooctene-modified bispecific antibody to the subject, contact between the tetrazine of the support composition and the trans-cyclooctene-modified bispecific antibody may occur, such that the binding agent and its complementary binding agent bind to one another to form a support complex, thereby selectively delivering the bispecific antibody to the target location in the subject. In some embodiments, selective delivery of the trans-cyclooctene-modified bispecific antibody results in a concentration of the bispecific at the target location that is greater than the concentration elsewhere in the subject (e.g., at non-targeted areas in the subject).

Provided herein is a method of treating cancer comprising administering to a subject in need thereof, a therapeutically effective amount of a trans-cyclooctene-modified bispecific antibody as described herein, or a pharmaceutically acceptable salt thereof, and a tetrazine activator.

In some embodiments, the cancer is metastatic. In some embodiments the cancer is melanoma, renal cancer, prostate cancer, ovarian cancer, endometrial carcinoma, breast cancer, glioblastoma, lung cancer, soft tissue sarcoma, fibrosarcoma, osteosarcoma, pancreatic cancer, gastric carcinoma, squamous cell carcinoma of head/neck, anal/vulvar carcinoma, esophageal carcinoma, pancreatic adenocarcinoma, cervical carcinoma, hepatocellular carcinoma, Kaposi's sarcoma, Non-Hodgkin lymphoma, Hodgkin's lymphoma, Wilm's tumor/neuroblastoma, bladder cancer, thyroid adenocarcinoma, pancreatic neuroendocrine tumors, prostatic adenocarcinoma, nasopharyngeal carcinoma, or cutaneous T-cell lymphoma.

In certain embodiments, the approach can be used for the treatment and/or diagnosis of hematological malignancies such as myelodysplastic syndromes, acute myeloid leukemia, myeldysplastic syndromes, chronic myelogenous leukemia, chronic myelomonocytic leukemia, primary myelofibrosis, diffuse large B-cell lymphoma, chronic lymphocytic leukemia, monoclonal gammopathy, plasma cell myeloma, follicular lymphoma, marginal zone lymphoma, classical Hodgkin lymphoma, monoclonal B-cell lymphocytosis, lymphoproliferative disorder NOS, T-cell lymphoma, precursor B-lymphoblastic leukemia, mantle cell lymphoma, plasmacytoma, Burkitt lymphoma, T-cell leukemia, hairy-cell leukemia, precursor T-lymphoblastic leukemia, nodular lymphocyte predominant Hodgkin lymphoma, as well as others.

In some embodiments, the cancer is a melanoma, renal cancer, prostate cancer, ovarian cancer, breast cancer, glioma, lung cancer, soft tissue carcinoma, soft tissue sarcoma, osteosarcoma, or pancreatic cancer.

In some embodiments, the cancer is a solid tumor.

In some embodiments, the cancer is a soft tissue sarcoma.

In some embodiments, the soft tissue sarcoma is a fibrosarcoma, rhabdomyosarcoma, or Ewing's sarcoma.

In some embodiments, the method also comprises enhancing or eliciting an immune response. In some embodiments the immune response is an increase in one or more of leukocytes, lymphocytes, monocytes, and eosinophils.

In some embodiments, the method further comprising administering a therapeutically effective amount of an additional therapeutic agent selected from the group consisting of an anticancer agent, an immunomodulatory agent, or a trans-cyclooctene prodrug thereof. Anticancer agents, immunomodulatory agents, and their trans-cyclooctene prodrugs are known in the art.

Indications for this approach include cancer, both hematological and solid cancers. In certain embodiments, the approach can be used for the treatment and/or diagnosis of soft tissue sarcomas: rhabdomyosarcoma, fibrosarcoma, Ewing's sarcoma, and all the different subtypes of soft tissue sarcoma as well as osteosarcoma. The compositions can be for the treatment and/or diagnosis of pigmented vilonodular synovitis.

Compositions comprising the trans-cyclooctene-modified bispecific antibody of the present disclosure find use in treatment and/or diagnosis of a condition or disease in a subject that is amenable to treatment or diagnosis by administration of a bispecific antibody. By “treatment” is meant that at least an amelioration of the symptoms associated with the condition afflicting the subject is achieved, where amelioration is used in a broad sense to refer to at least a reduction in the magnitude of a parameter, e.g., symptom, associated with the condition being treated. As such, treatment also includes situations where the pathological condition, or at least symptoms associated therewith, are completely inhibited, e.g., prevented from happening, or stopped, e.g., terminated, such that the subject no longer suffers from the condition, or at least the symptoms that characterize the condition. Treatment may include inhibition, that is, arresting the development or further development of clinical symptoms, e.g., mitigating or completely inhibiting an active disease. Treatment may include relief, that is, causing the regression of clinical symptoms. For example, in the context of cancer, the term “treating” includes any or all of: reducing growth of a solid tumor, inhibiting replication of cancer cells, reducing overall tumor burden, prolonged survival and ameliorating one or more symptoms associated with a cancer.

The subject to be treated can be one that is in need of therapy, where the subject to be treated is one amenable to treatment using a bispecific antibody. Accordingly, a variety of subjects may be amenable to treatment as disclosed herein. Generally, such subjects are “mammals,” with humans being of interest. Other subjects can include domestic pets (e.g., dogs and cats), livestock (e.g., cows, pigs, goats, horses, and the like), rodents (e.g., mice, guinea pigs, and rats, e.g., as in animal models of disease), as well as non-human primates (e.g., chimpanzees, and monkeys).

In certain embodiments, the trans-cyclooctene-modified bispecific antibodies as described herein can be used for the treatment, prevention, and/or diagnosis of solid tumors, including but not limited to, melanoma (e.g., unresectable, metastatic melanoma), renal cancer (e.g., renal cell carcinoma), prostate cancer (e.g., metastatic castration resistant prostate cancer), ovarian cancer (e.g., epithelial ovarian cancer, such as metastatic epithelial ovarian cancer), endometrial carcinoma, breast cancer (e.g., triple negative breast cancer), glioblastoma (e.g., glioblastoma multiforme), and lung cancer (e.g., non-small cell lung cancer), soft tissue sarcoma, fibrosarcoma, osteosarcoma, pancreatic cancer, gastric carcinoma, squamous cell carcinoma of head/neck, anal/vulvar carcinoma, esophageal carcinoma, pancreatic adenocarcinoma, cervical carcinoma, hepatocellular carcinoma, Kaposi's sarcoma, Non-Hodgkin lymphoma, Hodgkin lymphoma, Wilm's tumor/neuroblastoma, bladder cancer, thyroid adenocarcinoma, pancreatic neuroendocrine tumors, prostatic adenocarcinoma, nasopharyngeal carcinoma, cutaneous T-cell lymphoma, among others. The disclosed approach lends itself well as an adjuvant/neoadjuvant system. For example, particles as disclosed herein could be placed during the biopsy, once the results from the study come back, the practitioner could deliver the appropriate cocktail to the desired site in the body. This would minimize the size of the tumor particularly in the context of a surgically resectable tumor. Then at the end of the surgery, the surgeon could place more particles around the surgical cavity and treat the patient with further doses of treatment (e.g. chemotherapy through the disclosed approach) to minimize the risk of any cancer cells that may have been missed in the surgical margins.

### Cancer

The disclosed methods may be used to treat or prevent cancer, including metastatic cancer. Cancer is a group of related diseases that may include sustained proliferative signaling, evasion of growth suppressors, resistance to cell death, enablement of replicative immortality, induction of angiogenesis, and the activation of invasion and metastasis. The disclosed methods may enhance or elicit an immune response against a cancer in the subject. The immune response may lead to an increase in one or more of leukocytes, lymphocytes, monocytes, and eosinophils.

Cancer that may be treated by the disclosed methods, includes, but is not limited to, astrocytoma, adrenocortical carcinoma, appendix cancer, basal cell carcinoma, bile duct cancer, bladder cancer, bone cancer, brain cancer, brain stem cancer, brain stem glioma, breast cancer, cervical cancer, colon cancer, colorectal cancer, cutaneous T-cell lymphoma, diffuse intrinsic pontine glioma, ductal cancer, endometrial cancer, ependymoma, Ewing's sarcoma, esophageal cancer, eye cancer, fibrosarcoma, gallbladder cancer, gastric cancer, gastrointestinal cancer, germ cell tumor, glioma, hepatocellular cancer, histiocytosis, Hodgkin lymphoma, hypopharyngeal cancer, intraocular melanoma, Kaposi sarcoma, kidney cancer, laryngeal cancer, leukemia, liver cancer, lung cancer, lymphoma, macroglobulinemia, melanoma, mesothelioma, mouth cancer, multiple myeloma, nasopharyngeal cancer, neuroblastoma, non-Hodgkin lymphoma, osteosarcoma, ovarian cancer, pancreatic cancer, parathyroid cancer, penile cancer, pharyngeal cancer, pituitary cancer, prostate cancer, rectal cancer, renal cell cancer, retinoblastoma, rhabdomyosarcoma, sarcoma, skin cancer, small cell lung cancer, small intestine cancer, soft tissue carcinoma, soft tissue sarcoma, solid tumor, squamous cell carcinoma, stomach cancer, T-cell lymphoma, testicular cancer, throat cancer, thymoma, thyroid cancer, trophoblastic tumor, urethral cancer, uterine cancer, uterine sarcoma, vaginal cancer, vulvar cancer, and Wilms tumor.

In some embodiments, the cancer that may be treated by the disclosed methods is melanoma, renal cancer, prostate cancer, ovarian cancer, breast cancer, glioma, lung cancer, soft tissue carcinoma, soft tissue sarcoma, osteosarcoma, or pancreatic cancer. In some embodiments, the cancer is a solid tumor. In some embodiments, the cancer is a soft tissue carcinoma. In some embodiments, the cancer is afibrosarcoma. In some embodiments, the cancer is diffuse intrinsic pontine glioma. In some embodiments, the cancer is a metastatic cancer.

Without being bound by a particular theory, local release of certain anti-cancer agents using the compounds and methods of the invention may produce or contribute to immunogenic cell death (ICD). For example, certain anti-cancer agents (e.g., anthracyclines, cyclophosphamide, oxaliplatin) have been reported to induce ICD. Kroemer et al. Annu. Rev. Immunol. 2013 (31), 51-72. Immunogenic apoptosis of cancer cells can induce an effective antitumor immune response through activation of dendritic cells (DCs) and consequent activation of specific T cell response. ICD is characterized by secretion of damage-associated molecular patterns (DAMPs). Three important DAMPs which are exposed to the cell surface during ICD. Calreticulin (CRT), one of the DAMP molecules, which is normally in the lumen of endoplasmic reticulum (ER), is translocated after the induction of immunogenic apoptosis to the surface of dying cell where it functions as an “eat me” signal for professional phagocytes. Other important surface exposed DAMPs are heat-shock proteins (HSPs), namely HSP70 and HSP90, which are under stress condition also translocated to the plasma membrane. On the cell surface they have an immunostimulatory effect, based on their interaction with number of antigen-presenting cell (APC) surface receptors like CD91 and CD40 and also facilitate crosspresentation of antigens derived from tumor cells on MHC class I molecule, which than leads to the CD8+ T cell response. Other important DAMPs, characteristic for ICD are secreted amphoterin (HMGB1) and ATP. HMGB1 is considered to be late apoptotic marker and its release to the extracellular space seems to be required for the optimal release and presentation of tumor antigens to dendritic cells. It binds to several pattern recognition receptors (PRRs) such as Toll-like receptor (TLR) 2 and 4, which are expressed on APCs. The most recently found DAMP released during immunogenic cell death is ATP, which functions as a “find-me” signal for monocytes when secreted and induces their attraction to the site of apoptosis. Kroemer et. al. Curr. Op. Immunol. 2008 (20), 504-511.

Thus, local release of ICD inducers using the compounds and methods of the invention may be beneficially combined with one or more immunomodulatory agents.

In certain embodiments, the methods can be used for the treatment, prevention, and/or diagnosis of solid tumors, including but not limited to, melanoma (e.g., unresectable, metastatic melanoma), renal cancer (e.g., renal cell carcinoma), prostate cancer (e.g., metastatic castration resistant prostate cancer), ovarian cancer (e.g., epithelial ovarian cancer, such as metastatic epithelial ovarian cancer), breast cancer (e.g., triple negative breast cancer), glioblastoma (e.g., glioblastoma multiforme), and lung cancer (e.g., non-small cell lung cancer), soft tissue sarcoma, fibrosarcoma, osteosarcoma, pancreatic cancer, among others.

The disclosed methods may minimize the size of the tumor prior to surgical resection. This would minimize the size of the tumor particularly in the context of a surgically resectable tumor. The disclosed trans-cyclooctene-modified bispecific antibodies, or a system using the same, may be administered during surgical resection. The disclosed trans-cyclooctene-modified bispecific antibodies, or a system using the same, may be administered after surgical resection.

A therapeutic support composition as disclosed herein may be placed around the surgical cavity at the end of surgical resection and the subject may then be treated with further doses of a trans-cyclooctene-modified bispecific antibody to minimize the risk of any cancer cells that may have been missed in the surgical margins.

In certain embodiments, the trans-cyclooctene-modified bispecific antibody and/or tetrazine activator may be administered endoscopically.

The disclosed methods may include multiple systemic doses of trans-cyclooctene-modified bispecific antibody that focus at one location.

The disclosed methods may be used to deliver a trans-cyclooctene-modified bispecific antibody to the site of a diffuse intrinsic pontine glioma (DIPG). The disclosed methods may include delivering a trans-cyclooctene-modified bispecific antibody systemically that are only activated at the tumor site. The disclosed methods may be used as a neoadjuvant or adjuvant therapy.

### Modes of Administration

Methods of treatment may include any number of modes of administration. Modes of administration may include tablets, pills, dragees, hard and soft gel capsules, granules, pellets, skin patches, skin creams, skin gels, aqueous, lipid, oily or other solutions, emulsions such as oil-in-water emulsions, liposomes, aqueous or oily suspensions, syrups, elixirs, solid emulsions, solid dispersions or dispersible powders.

For parenteral administration, a trans-cyclooctene-modified bispecific antibody or composition comprising the same as disclosed herein disclosed herein may be dissolved or suspended in a physiologically acceptable diluent, such as water, buffer, oils with or without solubilizers, surface-active agents, dispersants or emulsifiers. Suitable oils may include, for example, olive oil, peanut oil, cottonseed oil, soybean oil, castor oil and sesame oil. For parenteral administration, a trans-cyclooctene-modified bispecific antibody or composition comprising the same as disclosed herein may be administered in the form of an aqueous, lipid, oily or other kind of solution or suspension, or even administered in the form of liposomes or nano-suspensions.

The term “parenterally,” as used herein, refers to modes of administration which include intravenous, intramuscular, intraperitoneal, intrasternal, subcutaneous and intraarticular injection and infusion.

Therapeutic support compositions may be administered locally at the site of a tumor, such as by injection or implantation. A trans-cyclooctene-modified bispecific antibody, or composition comprising the same, may be administered by any convenient route, in view of a subject's condition and judgment of medical professionals. Parenteral administration is a suitable means of administering a trans-cyclooctene-modified bispecific antibody or composition comprising the same.

The amount administered to a subject can be initially determined based on guidance of a dose and/or dosage regimen. In general, the trans-cyclooctene-modified bispecific antibody or composition comprising the same as disclosed herein can provide for targeted delivery and/or enhanced serum half-life of the bound drug, thus providing for at least one of reduced dose or reduced administrations in a dosage regimen. Thus, the trans-cyclooctene-modified bispecific antibody or composition comprising the same as disclosed herein can provide for reduced dose and/or reduced administration in a dosage regimen relative to the bispecific antibody prior to being conjugated as described in the present disclosure.

A composition, or pharmaceutical formulation, may be provided in unit dosage form. In such form the pharmaceutical formulation may be subdivided into unit doses containing appropriate quantities of the compositions of the present disclosure. The unit dosage form can be a packaged preparation, the package containing discrete quantities of the preparation, such as packeted tablets, capsules, and powders in pouches, vials or ampoules.

In some embodiments, provided is a kit comprising a trans-cyclooctene-modified bispecific antibody or composition comprising the same as disclosed herein, and instructions for use thereof.

In some embodiments, the kit further comprises a tetrazine activator.

Compositions of the present disclosure can be present in any suitable amount, and can depend on various factors including, but not limited to, weight and age of the subject, state of the disease, etc. Suitable dosage ranges for the composition of the present disclosure include from 0.1 mg to 10,000 mg, or 1 mg to 1000 mg, or 10 mg to 750 mg, or 25 mg to 500 mg, or 50 mg to 250 mg. For instance, suitable dosages for the composition of the present disclosure include 1 mg, 5 mg, 10 mg, 20 mg, 30 mg, 40 mg, 50 mg, 60 mg, 70 mg, 80 mg, 90 mg, 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg or 1000 mg.

In some embodiments, multiple doses are administered. The frequency of administration can vary depending on any of a variety of factors, e.g., severity of the symptoms, condition of the subject, etc. For example, in some embodiments, a trans-cyclooctene-modified bispecific antibody or composition comprising the same as disclosed herein is administered once per month, twice per month, three times per month, every other week (qow), once per week (qw), twice per week (biw), three times per week (tiw), four times per week, five times per week, six times per week, every other day (qod), daily (qd), twice a day (qid), or three times a day (tid).

The trans-cyclooctene-modified bispecific antibody or composition comprising the same as disclosed herein can be administered at any suitable frequency, interval and duration. For example, a trans-cyclooctene-modified bispecific antibody or composition comprising the same as disclosed herein can be administered once an hour, or two, three or more times an hour, once a day, or two, three, or more times per day, or once every 2 days, 3 days, 4 days, 5 days, 6 days, or 7 days, so as to provide the desired dosage level to the subject. When the trans-cyclooctene-modified bispecific antibody or composition comprising the same as disclosed herein is administered more than once a day, representative intervals include 5 min, 10 min, 15 min, 20 min, 30 min, 45 min and 60 minutes, as well as 1 hr, 2 hr, 4 hr, 6 hr, 8 hr, 10 hr, 12 hr, 16 hr, 20 hr, and 24 hours. Administration can be administered once, twice, or three or more times, for an hour, for 1 to 6 hours, for 1 to 12 hours, for 1 to 24 hours, for 6 to 12 hours, for 12 to 24 hours, for a single day, for 1 to 7 days, for a single week, for 1 to 4 weeks, for a month, for 1 to 12 months, for a year or more, or even indefinitely.

The trans-cyclooctene-modified bispecific antibody or composition comprising the same as disclosed herein of the present disclosure can be co-administered with another active agent. Co-administration includes administering the trans-cyclooctene-modified bispecific antibody or composition comprising the same and active agent within 0.5 hr, 1 hr, 2 hr, 4 hr, 6 hr, 8 hr, 10 hr, 12 hr, 16 hr, 20 hr, or 24 hours of each other. Co-administration also includes administering simultaneously or approximately simultaneously (e.g., within about 1 min, 5 min, 10 min, 15 min, 20 min, or 30 minutes of each other), or sequentially in any order. In addition, the trans-cyclooctene-modified bispecific antibody or composition comprising the same and the active agent can each be administered once a day, or two, three, or more times per day so as to provide the desired dosage level per day.

Co-administration can be accomplished by coimplantation or coinjection.

In some embodiments, co-administration can be accomplished by co-formulation, e.g., preparing a single pharmaceutical formulation including both the trans-cyclooctene-modified bispecific antibody or composition comprising the same and the active agent. In other embodiments, the trans-cyclooctene-modified bispecific antibody or composition comprising the same and the active agent can be formulated separately and co-administered to the subject.

The trans-cyclooctene-modified bispecific antibody or composition comprising the same and the active agent can be present in a formulation in any suitable weight ratio, such as from 1:100 to 100:1 (w/w), or 1:50 to 50:1, or 1:25 to 25:1, or 1:10 to 10:1, or 1:5 to 5:1 (w/w). The trans-cyclooctene-modified bispecific antibody or composition comprising the same and the other active agent can be present in any suitable weight ratio, such as 1:100 (w/w), 1:75, 1:50, 1:25, 1:10, 1:5, 1:4, 1:3, 1:2, 1:1, 2:1, 3:1, 4:1, 5:1, 10:1, 25:1, 50:1, 75:1, or 100:1 (w/w).

### Combination Therapies

In one aspect, the invention provides a method of treating cancer or enhancing or eliciting an immune response comprising administering to a subject in need thereof: a therapeutically effective amount of a trans-cyclooctene-modified bispecific antibody as described herein; a tetrazine activator as described herein; and a therapeutically effective amount of an additional therapeutic agent selected from the group consisting of an anticancer agent, an immunomodulatory agent, or a trans-cyclooctene prodrug thereof.

The disclosure also provides a pharmaceutical combination comprising a trans-cyclooctene-modified bispecific antibody described herein, or composition thereof; a tetrazine activator, as described herein; and an additional therapeutic agent selected from the group consisting of an anticancer agent, an immunomodulatory agent, or a trans-cyclooctene prodrug thereof, for use in the treatment or prevention of a cancer or for use in enhancing or eliciting an immune response.

The disclosure also provides the use of a pharmaceutical combination comprising a trans-cyclooctene-modified bispecific antibody described herein, or composition thereof; a tetrazine activator; and a therapeutically effective amount of an additional therapeutic agent selected from the group consisting of an anticancer agent, an immunomodulatory agent, or a trans-cyclooctene prodrug thereof for the treatment or prevention of a cancer or for use in enhancing or eliciting an immune response.

In the methods and uses described herein, the components of the pharmaceutical combinations may be administered/used simultaneously, separately, or sequentially, and in any order, and the components may be administered separately or as a fixed combination. For example, the delay of progression or treatment of diseases according to the invention may comprise administration of the first active ingredient in free or pharmaceutically acceptable salt form and administration of the second active ingredient in free or pharmaceutically acceptable salt form, simultaneously or sequentially in any order, in jointly therapeutically effective amounts or effective amounts, e.g. in daily dosages corresponding to the amounts described herein. The individual active ingredients of the combination can be administered separately at different times during the course of therapy or concurrently in divided or single dosage forms. The instant disclosure is therefore to be understood as embracing all such regimes of simultaneous or alternating treatment and the term “administering” is to be interpreted accordingly. Thus, a pharmaceutical combination, as used herein, defines either a fixed combination in one dosage unit form or separate dosages forms for the combined administration where the combined administration may be independently at the same time or at different times. As a further example, the trans-cyclooctene-modified bispecific antibody and tetrazine activator may be administered/used simultaneously (e.g., through coinjection or coimplantation), separately, or sequentially, followed by administration of an additional therapeutic agent selected from the group consisting of an anticancer agent, an immunomodulatory agent, or a trans-cyclooctene prodrug.

In certain embodiments, the methods and uses in treating cancer include administering/localizing the trans-cyclooctene-modified bispecific antibody at a tumor. The administration of the trans-cyclooctene-modified bispecific antibody, or composition thereof; the tetrazine activator; and the additional therapeutic agent may inhibit the growth of the tumor.

Additional therapeutic agent(s) may be administered simultaneously or sequentially with the disclosed trans-cyclooctene-modified bispecific antibody. Sequential administration includes administration before or after the disclosed trans-cyclooctene-modified bispecific antibody. An additional therapeutic agent may be administered before the disclosed trans-cyclooctene-modified bispecific antibody. An additional therapeutic agent may be administered after the disclosed trans-cyclooctene-modified bispecific antibody. An additional therapeutic agent may be administered at the same time as the disclosed trans-cyclooctene-modified bispecific antibody. In some embodiments, the additional therapeutic agent or agents may be administered in the same composition as the disclosed trans-cyclooctene-modified bispecific antibody. In other embodiments, there may be an interval of time between administration of the additional therapeutic agent and the disclosed trans-cyclooctene-modified bispecific antibody. In some embodiments, administration of an additional therapeutic agent with a disclosed trans-cyclooctene-modified bispecific antibody may allow lower doses of the other therapeutic agents and/or administration at less frequent intervals. When used in combination with one or more other active ingredients, the trans-cyclooctene-modified bispecific antibody of the present invention and the other active ingredients may be used in lower doses than when each is used singly. Accordingly, the pharmaceutical compositions of the present invention include those that contain one or more other active ingredients, in addition to a trans-cyclooctene-modified bispecific antibody of the present disclosure.

### Anticancer Agents

Exemplary anti-cancer agents include, but are not limited to, Abiraterone Acetate, Abitrexate (Methotrexate), Abraxane (Paclitaxel Albumin-stabilized Nanoparticle Formulation), ABVD, ABVE, ABVE-PC, AC, AC-T, Adcetris (Brentuximab Vedotin), ADE, Ado-Trastuzumab Emtansine, Adriamycin (Doxorubicin Hydrochloride), Adrucil (Fluorouracil), Afatinib Dimaleate, Afinitor (Everolimus), Aldara (Imiquimod), Aldesleukin, Alemtuzumab, Alimta (Pemetrexed Disodium), Aloxi (Palonosetron Hydrochloride), Ambochlorin (Chlorambucil), Aminolevulinic Acid, Anastrozole, Aprepitant, Aredia (Pamidronate Disodium), Arimidex (Anastrozole), Aromasin (Exemestane), Arranon (Nelarabine), Arsenic Trioxide, Arzerra (Ofatumumab), Asparaginase Erwinia chrysanthemi, Avastin (Bevacizumab), Axitinib, Azacitidine, BEACOPP, Bendamustine Hydrochloride, BEP, Bevacizumab, Bexarotene, Bexxar (Tositumomab and 1131 Iodine Tositumomab), Bicalutamide, Bleomycin, Bortezomib, Bosulif (Bosutinib), Bosutinib, Brentuximab Vedotin, Busulfan, Busulfex (Busulfan), Cabazitaxel, Cabozantinib-S-Malate, CAF, Campath (Alemtuzumab), Camptosar (Irinotecan Hydrochloride), Capecitabine, CAPOX, Carboplatin, Carboplatin-Taxol, Carfilzomib, Casodex (Bicalutamide), CeeNU (Lomustine), Cerubidine (Daunorubicin Hydrochloride), Cervarix (Recombinant HPV Bivalent Vaccine), Cetuximab, Chlorambucil, Chlorambucil-Prednisone, CHOP, Cisplatin, Clafen (Cyclophosphamide), Clofarabine, Clofarex (Clofarabine), Clolar (Clofarabine), CMF, Cometriq (Cabozantinib-S-Malate), COPP, COPP-ABV, Cosmegen (Dactinomycin), Crizotinib, CVP, Cyclophosphamide, Cyfos (Ifosfamide), Cytarabine, Cytarabine liposomal, Cytosar-U (Cytarabine), Cytoxan (Cyclophosphamide), Dabrafenib, Dacarbazine, Dacogen (Decitabine), Dactinomycin, Dasatinib, Daunorubicin Hydrochloride, Decitabine, Degarelix, Denileukin Diftitox, Denosumab, DepoCyt (Liposomal Cytarabine), DepoFoam (Liposomal Cytarabine), Dexrazoxane Hydrochloride, Docetaxel, Doxil (Doxorubicin Hydrochloride Liposome), Doxorubicin Hydrochloride, Doxorubicin Hydrochloride Liposome, Dox-SL (Doxorubicin Hydrochloride Liposome), DTIC-Dome (Dacarbazine), Efudex (Fluorouracil), Elitek (Rasburicase), Ellence (Epirubicin Hydrochloride), Eloxatin (Oxaliplatin), Eltrombopag Olamine, Emend (Aprepitant), Enzalutamide, Epirubicin Hydrochloride, EPOCH, Erbitux (Cetuximab), Eribulin Mesylate, Erivedge (Vismodegib), Erlotinib Hydrochloride, Erwinaze (Asparaginase Erwinia chrysanthemi), Etopophos (Etoposide Phosphate), Etoposide, Etoposide Phosphate, Evacet (Doxorubicin Hydrochloride Liposome), Everolimus, Evista (Raloxifene Hydrochloride), Exemestane, Fareston (Toremifene), Faslodex (Fulvestrant), FEC, Femara (Letrozole), Filgrastim, Fludara (Fludarabine Phosphate), Fludarabine Phosphate, Fluoroplex (Fluorouracil), Fluorouracil, Folex (Methotrexate), Folex PFS (Methotrexate), Folfiri, Folfiri-Bevacizumab, Folfiri-Cetuximab, Folfirinox, Folfox (Leucovorin, Fluorouracil, Oxaliplatin), Folotyn (Pralatrexate), FU-LV, Fulvestrant, Gardasil (Recombinant HPV Quadrivalent Vaccine), Gazyva (Obinutuzumab), Gefitinib, Gemcitabine Hydrochloride, Gemcitabine-Cisplatin, Gemcitabine-Oxaliplatin, Gemtuzumab Ozogamicin, Gemzar (Gemcitabine Hydrochloride), Gilotrif (Afatinib Dimaleate), Gleevec (Imatinib Mesylate), Glucarpidase, Goserelin Acetate, Halaven (Eribulin Mesylate), Herceptin (Trastuzumab), HPV Bivalent Vaccine, Recombinant, HPV Quadrivalent Vaccine, Recombinant, Hycamtin (Topotecan Hydrochloride), Hyper-CVAD, Ibritumomab Tiuxetan, Ibrutinib, ICE, Iclusig (Ponatinib Hydrochloride), Ifex (Ifosfamide), Ifosf amide, Ifosfamidum (Ifosfamide), Imatinib Mesylate, Imbruvica (Ibrutinib), Imiquimod, Inlyta (Axitinib), Intron A (Recombinant Interferon Alfa-2b), Iodine 131 Tositumomab and Tositumomab, Ipilimumab, Iressa (Gefitinib), Irinotecan Hydrochloride, Istodax (Romidepsin), Ixabepilone, Ixempra (Ixabepilone), Jakafi (Ruxolitinib Phosphate), Jevtana (Cabazitaxel), Kadcyla (Ado-Trastuzumab Emtansine), Keoxifene (Raloxifene Hydrochloride), Kepivance (Palifermin), Kyprolis (Carfilzomib), Lapatinib Ditosylate, Lenalidomide, Letrozole, Leucovorin Calcium, Leukeran (Chlorambucil), Leuprolide Acetate, Levulan (Aminolevulinic Acid), Linfolizin (Chlorambucil), LipoDox (Doxorubicin Hydrochloride Liposome), Liposomal Cytarabine, Lomustine, Lupron (Leuprolide Acetate), Lupron Depot (Leuprolide Acetate), Lupron Depot-Ped (Leuprolide Acetate), Lupron Depot-3 Month (Leuprolide Acetate), Lupron Depot-4 Month (Leuprolide Acetate), Marqibo (Vincristine Sulfate Liposome), Matulane (Procarbazine Hydrochloride), Mechlorethamine Hydrochloride, Megace (Megestrol Acetate), Megestrol Acetate, Mekinist (Trametinib), Mercaptopurine, Mesna, Mesnex (Mesna), Methazolastone (Temozolomide), Methotrexate, Methotrexate LPF (Methotrexate), Mexate (Methotrexate), Mexate-AQ (Methotrexate), Mitomycin C, Mitozytrex (Mitomycin C), MOPP, Mozobil (Plerixafor), Mustargen (Mechlorethamine Hydrochloride), Mutamycin (Mitomycin C), Myleran (Busulfan), Mylosar (Azacitidine), Mylotarg (Gemtuzumab Ozogamicin), Nanoparticle Paclitaxel (Paclitaxel Albumin-stabilized Nanoparticle Formulation), Navelbine (Vinorelbine Tartrate), Nelarabine, Neosar (Cyclophosphamide), Neupogen (Filgrastim), Nexavar (Sorafenib Tosylate), Nilotinib, Nolvadex (Tamoxifen Citrate), Nplate (Romiplostim), Obinutuzumab, Ofatumumab, Omacetaxine Mepesuccinate, Oncaspar (Pegaspargase), Ontak (Denileukin Diftitox), OEPA, OPPA, Oxaliplatin, Paclitaxel, Paclitaxel Albumin-stabilized Nanoparticle Formulation, Palifermin, Palonosetron Hydrochloride, Pamidronate Disodium, Panitumumab, Paraplat (Carboplatin), Paraplatin (Carboplatin), Pazopanib Hydrochloride, Pegaspargase, Peginterferon Alfa-2b, PEG-Intron (Peginterferon Alfa-2b), Pemetrexed Disodium, Perjeta (Pertuzumab), Pertuzumab, Platinol (Cisplatin), Platinol-AQ (Cisplatin), Plerixafor, Pomalidomide, Pomalyst (Pomalidomide), Ponatinib Hydrochloride, Pralatrexate, Prednisone, Procarbazine Hydrochloride, Proleukin (Aldesleukin), Prolia (Denosumab), Promacta (Eltrombopag Olamine), Provenge (Sipuleucel-T), Purinethol (Mercaptopurine), Radium 223 Dichloride, Raloxifene Hydrochloride, Rasburicase, R-CHOP, R-CVP, Recombinant HPV Bivalent Vaccine, Recombinant HPV Quadrivalent Vaccine, Recombinant Interferon Alfa-2b, Regorafenib, Revlimid (Lenalidomide), Rheumatrex (Methotrexate), Rituxan (Rituximab), Rituximab, Romidepsin, Romiplostim, Rubidomycin (Daunorubicin Hydrochloride), Ruxolitinib Phosphate, Sclerosol Intrapleural Aerosol (Talc), Sipuleucel-T, Sorafenib Tosylate, Sprycel (Dasatinib), Stanford V, Sterile Talc Powder (Talc), Steritalc (Talc), Stivarga (Regorafenib), Sunitinib Malate, Sutent (Sunitinib Malate), Sylatron (Peginterferon Alfa-2b), Synovir (Thalidomide), Synribo (Omacetaxine Mepesuccinate), Tafinlar (Dabrafenib), Talc, Tamoxifen Citrate, Tarabine PFS (Cytarabine), Tarceva (Erlotinib Hydrochloride), Targretin (Bexarotene), Tasigna (Nilotinib), Taxol (Paclitaxel), Taxotere (Docetaxel), Temodar (Temozolomide), Temozolomide, Temsirolimus, Thalidomide, Thalomid (Thalidomide), Toposar (Etoposide), Topotecan Hydrochloride, Toremifene, Torisel (Temsirolimus), Tositumomab and 1 131 Iodine Tositumomab, Totect (Dexrazoxane Hydrochloride), Trametinib, Trastuzumab, Treanda (Bendamustine Hydrochloride), Trisenox (Arsenic Trioxide), Tykerb (Lapatinib Ditosylate), Vandetanib, VAMP, Vectibix (Panitumumab), VelP, Velban (Vinblastine Sulfate), Velcade (Bortezomib), Velsar (Vinblastine Sulfate), Vemurafenib, VePesid (Etoposide), Viadur (Leuprolide Acetate), Vidaza (Azacitidine), Vinblastine Sulfate, Vincasar PFS (Vincristine Sulfate), Vincristine Sulfate, Vincristine Sulfate Liposome, Vinorelbine Tartrate, Vismodegib, Voraxaze (Glucarpidase), Vorinostat, Votrient (Pazopanib Hydrochloride), Wellcovorin (Leucovorin Calcium), Xalkori (Crizotinib), Xeloda (Capecitabine), Xelox, Xgeva (Denosumab), Xofigo (Radium 223 Dichloride), Xtandi (Enzalutamide), Yervoy (Ipilimumab), Zaltrap (Ziv-Aflibercept), Zelboraf (Vemurafenib), Zevalin (Ibritumomab Tiuxetan), Zinecard (Dexrazoxane Hydrochloride), Ziv-Aflibercept, Zoladex (Goserelin Acetate), Zoledronic Acid, Zolinza (Vorinostat), Zometa (Zoledronic Acid), and Zytiga (Abiraterone Acetate).

The anticancer agent may be a PBD dimer, calicheamicin, speromycin, tubulysin B, rhizoxin, dolastatin, didemnin B, camptothecin, CBI, temsirolimus, actinomycin D, epothilone B, taxol, cryptophycin, SN38, velcade, bruceantin, DAVLBH, DM1, Phyllanthoside, Alimta, T2 Toxin, MMC, vantalanib, vinorelbine, brefeldin, sunitinib, daunomycin, semaxanib, tarceva, iressa, irinotecan, LY-541503, geldanomycin, gemcitabine, methotrexate, gleevec, topotecan, bleomycin, doxorubicin, cisplatin, N-mustards, etoposide, or 5-FU.

In certain embodiments, an anticancer agent is an anthracycline. In certain embodiments, an anticancer agent is a taxane. In certain embodiments, an anticancer agent is gemcitabine. In certain embodiments, an anticancer agent is doxorubicin. In certain embodiments, an anticancer agent is docetaxel. In certain embodiments, an anticancer agent is SN38. In certain embodiments, an anticancer agent is monomethyl auristatin E. In certain embodiments, an anticancer agent is an alkylating agent, antimetabolite (folate antagonist, purine antagonist, pyrimidine antagonist), antibiotic, taxane, vinca alkaloid, or campothecin analogue.

### E. Synthetic Methods

The trans-cyclooctene-modified bispecific antibodies may be prepared using the methods disclosed herein and routine modifications thereof, which will be apparent given the disclosure herein and methods well known in the art. Conventional and well-known synthetic methods may be used in addition to the teachings herein. The synthesis of typical trans-cyclooctene-modified bispecific antibodies described herein may be accomplished as described in the following examples. If available, reagents and starting materials may be purchased commercially, e.g., from Sigma Aldrich or other chemical suppliers.

It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvent used, but such conditions can be determined by one skilled in the art by routine optimization procedures.

Additionally, conventional protecting groups may be necessary to prevent certain functional groups from undergoing undesired reactions. Suitable protecting groups for various functional groups as well as suitable conditions for protecting and deprotecting particular functional groups are well known in the art. For example, numerous protecting groups are described in Wuts, P. G. M., Greene, T. W., & Greene, T. W. (2006). Greene's protective groups in organic synthesis. Hoboken, N.J., Wiley-Interscience, and references cited therein.

Furthermore, the trans-cyclooctene-modified bispecific antibodies of this disclosure may contain one or more chiral centers. Accordingly, if desired, such trans-cyclooctene-modified bispecific antibodies can be prepared or isolated as pure stereoisomers, i.e., as individual enantiomers or diastereomers or as stereoisomer-enriched mixtures. All such stereoisomers (and enriched mixtures) are included within the scope of this disclosure, unless otherwise indicated. Pure stereoisomers (or enriched mixtures) may be prepared using, for example, optically active starting materials or stereoselective reagents well-known in the art. Alternatively, racemic mixtures can be separated using, for example, chiral column chromatography, chiral resolving agents, and the like.

The starting materials for the following reactions are generally known compounds or can be prepared by known procedures or obvious modifications thereof. For example, many of the starting materials are available from commercial suppliers such as Aldrich Chemical Co. (Milwaukee, Wisconsin, USA), Bachem (Torrance, California, USA), Emka-Chemce or Sigma (St. Louis, Missouri, USA). Others may be prepared by procedures or obvious modifications thereof, described in standard reference texts such as Fieser and Fieser's Reagents for Organic Synthesis, Volumes 1-15 (John Wiley, and Sons, 1991), Rodd's Chemistry of Carbon Compounds, Volumes 1-5, and Supplementals (Elsevier Science Publishers, 1989) organic Reactions, Volumes 1-40 (John Wiley, and Sons, 1991), March's Advanced Organic Chemistry, (John Wiley, and Sons, 5th Edition, 2001), and Larock's Comprehensive Organic Transformations (VCH Publishers Inc., 1989).

### EXAMPLES

The following examples are included to demonstrate specific embodiments of the disclosure. It should be appreciated by those of skill in the art that the techniques disclosed in the examples which follow represent techniques to function well in the practice of the disclosure, and thus can be considered to constitute specific modes for its practice. However, those of skill in the art should, in light of the present disclosure, appreciate that many changes can be made in the specific embodiments which are disclosed and still obtain a like or similar result without departing from the spirit and scope of the disclosure.

### Example 1: Synthesis of TCO-Bispecific T Cell Engager (BiTE) Conjugate

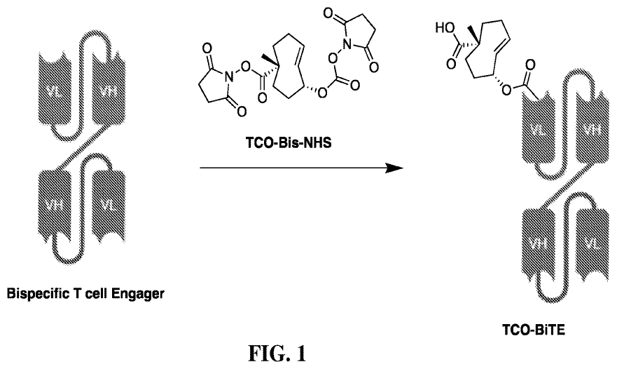

Preparation of TCO-BiTE conjugate. To blinatumomab (1 mg) in PBS, pH 7.4 (1 mL) at rt is added TCO-Bis-NHS (20 μL, 10 mM in DMSO; 10 equiv). The solution is mixed gently by inversion and aged at rt for 1 h. The reaction is quenched by adjusting the pH to 8.5 with Tris (1 M, pH 8.5) and aging at rt for 15 min. Per the manufacturer's instructions a NAP-10 desalting column is equilibrated with PBS. The reaction mixture is applied to the column and the product elutes with PBS (schematic shown in FIG. 1). The binding affinity of the conjugate can be tested using an ELISA assay.

### Example 2: Expression of TCO-Bispecific T Cell Engager (BiTE) Conjugate

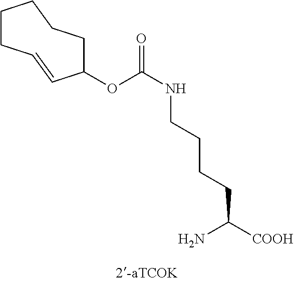 [SMILES: N[C@@H](CCCCNC(=O)OC1/C=C/CCCCC1)C(=O)O]

An amber codon is substituted in the blinatumomab gene at one or more of the lysine residues at positions 279, 314, 339, 341, 343, 350, 431, 451, 457, 465, or 515. The axial trans-cyclooct-2-ene-lysine (2′-aTCOK)-containing antibody is recombinantly produced in suspension Chinese hamster ovary (CHO-K1) cells. The CHO-K1, in which an orthogonal Escherichia coli tyrosyl-derived tRNA/aaRS pair (Mm-PylRS-AF (Y306A, Y384F) derived from Methanosarcina mazei) that incorporates 2′-aTCOK is stably integrated into the genome using selectable markers. The mutant gene for the protein is incorporated into a plasmid mammalian expression. The plasmid is transiently transfected into the tRNA/aaRS-expressing CHO cell line. Folded protein is collected from media and purified by affinity chromatography. The binding affinity of the conjugate can be tested using an ELISA assay.

### Example 3: Expression of TCO-Bispecific T Cell Engager (BiTE) Conjugate

An amber codon is substituted in the blinatumomab gene at one or more of the lysine residues at positions 279, 314, 339, 341, 343, 350, 431, 451, 457, 465, or 515. The axial trans-cyclooct-2-ene-lysine (2′-aTCOK)-containing antibody is recombinantly produced in suspension Chinese hamster ovary (CHO-K1) cells. The CHO-K1, in which an orthogonal Escherichia coli tyrosyl-derived tRNA/aaRS pair (Mm-PylRS-AF (Y306A, Y384F) derived from Methanosarcina mazei) that incorporates 2′-aTCOK is stably integrated into the genome using selectable markers. The mutant gene for the protein is stably incorporated into the tRNA/aaRS-expressing CHO cell line. Folded protein is collected from media and purified by affinity chromatography. The binding affinity of the conjugate can be tested using an ELISA assay.

Unless otherwise defined, all technical and scientific terms used herein have the same meaning as commonly understood by one of ordinary skill in the art to which this invention belongs.

The inventions illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed.

All publications, patent applications, patents, and other references mentioned herein are expressly incorporated by reference in their entirety, to the same extent as if each were incorporated by reference individually. In case of conflict, the present specification, including definitions, will control.

It is to be understood that while the disclosure has been described in conjunction with the above embodiments, that the foregoing description and examples are intended to illustrate and not limit the scope of the disclosure. Other aspects, advantages and modifications within the scope of the disclosure will be apparent to those skilled in the art to which the disclosure pertains.

What is claimed is:

1. A trans-cyclooctene-modified bispecific antibody comprises a bispecific antibody portion having a first antigen target binding moiety and a second antigen target binding moiety, and the antibody portion further comprises at least one trans-cyclooctene moiety covalently bonded thereto optionally via a linker.

2. The trans-cyclooctene-modified bispecific antibody of claim 1, wherein the bispecific antibody comprises at least a portion of an antibody or antibody fragment that binds to an antigen on a hematopoietic cell.

3. The trans-cyclooctene-modified bispecific antibody of claim 1, wherein the bispecific antibody comprises at least a portion of an antibody or antibody fragment that binds to an antigen on a myeloid cell.

4. The trans-cyclooctene-modified bispecific antibody of claim 1, wherein the bispecific antibody comprises at least a portion of an antibody or antibody fragment that binds to an antigen on a lymphocyte.

5. The trans-cyclooctene-modified bispecific antibody of claim 1, wherein the bispecific antibody comprises at least a portion of an antibody or antibody fragment that binds to an antigen on a T-cell or T-cell progenitor.

6. The trans-cyclooctene-modified bispecific antibody of claim 1, wherein the bispecific antibody comprises at least a portion of an antibody or antibody fragment that binds to an antigen on a cancerous cell.

7. The trans-cyclooctene-modified bispecific antibody of claim 1, wherein the bispecific antibody portion is a CD3×HER2 bispecific antibody, a PD-L1×EGFR bispecific antibody, a CTLA-4×PD-1 bispecific antibody, or a PD-L1×4-1BB bispecific antibody, a IL-17A×IL-17F bispecific antibody, a VEGF×Ang2 bispecific antibody, a Nectin-4×TLR8 bispecific antibody, a PSMA×CD3 bispecific antibody, a BCMA×CD3 bispecific antibody, a FGFR1×KLB bispecific antibody, a DLL3×CD3 bispecific antibody, a CD38×CD3 bispecific antibody, a FcRL5×CD3 bispecific antibody, a GPRC5D×CD3 bispecific antibody, a GPC3×CD3 bispecific antibody, a HER2×HER3 bispecific antibody, a HER3×IGF-1R bispecific antibody, a EGFR x HER3 bispecific antibody, a CD123×CD3 bispecific antibody, a CD19×CD3 bispecific antibody, a EPCAM×CD3 bispecific antibody, a FIXa x FX bispecific antibody, a EGFR x cMET bispecific antibody.

8. The trans-cyclooctene-modified bispecific antibody of claim 1, wherein the bispecific antibody is AMF13, NXT007, LY3164530, JNJ-61186372, EMB01, MEHD7945A, RG7221, RG7386, ATN-103, TS-152, ALX-0061, AFM13, AK112, 1B1315, ABT122, RO7040547, SAR156597, AZ17, BI 836880, vanucizumab, ABT-165, OMP-305B83, TR009, EMB01, MCLA-158, ZW25, GTB-3550, TG-1801, XmAB23104, AK104, MGD019, XmAb20717, MEDI5752, MGD013, RG7769, LY3434172, FS118, KN046, LY3415244, AFM11, ERY947, RG7802, AMG420, AMG330, AMG212, AMG596, AMG552, AMG673, AMG427, AMG701, MGD006, MGD007, AMG424, MGD011, blinatumomab, AMG562, A-319, RG7828, REGN1979, AR6026, GEN3013, FBTA05, plamotamab, AMG330, AMG673, AMV-564, GEM333, GBR1342, AMG424, AMG420, AMG701, JNJ-64007957, EM801, PF-06863135, REGN5458, TNB-383B, APV0436, MGD006, Xmab14045, JNJ-63709178, MCLA-117, RG6160, AMG427, JNJ-64407564, AMG111, RG7802, solitomab, catumaxomab, pasotuxizumab, HPN-424, AMG160, MOR209, BAY2010112, ertumaxomab, GBR1302, H802, RG6294, PF06671008, MGD007, MGD009, AMG757, REGN4018, AMG596, ERY974, tidutamab, huGD2-BsAB, MGD014, M1095, BI 836880, SBT6290, CCW702, AMG420, CC-93269, PF-06863135, REGN5458, AMG701, TNB383B, BFKB8488A, BI 764532, AMG757, AMG424, GBR1342, BFCR4350A, JNJ-64407564, ERY974, MCLA-128, MM-111, istiratumab, duligotumab, flotatuzumab, XmAb14045, JNJ-63709178, blinatumomab, durotuxizumab, catumaxomab, emicizumab, JNJ-61186372, or amivantamab.

9. The trans-cyclooctene-modified bispecific antibody of claim 1, wherein the first antigen target binding moiety is an anti-CD3 antibody, anti-ALB antibody, anti-ANG-2 antibody, anti-CD16 antibody, anti-CD16A antibody, anti-CD47 antibody, anti-DLL4 antibody, anti-DLL5 antibody, anti-EGFR antibody, anti-FAP antibody, anti-FIXa antibody, anti-HER2 ec domain II antibody, anti-IL-17 antibody, anti-IL-23 antibody, anti-IL-4 antibody, anti-PD1 antibody, anti-PDL1 antibody, or anti-TNF-α antibody, or antibody fragment thereof.

10. The trans-cyclooctene-modified bispecific antibody of claim 1 or 7, wherein the second antigen target binding moiety is an anti-ANG2 antibody, anti-CD19 antibody, anti-CD30 antibody, anti-CD33 antibody, anti-cMET antibody, anti-CTLA4 antibody, anti-DR5 antibody, anti-FX antibody, anti-HER2 antibody, anti-HER2 ec domain IV antibody, anti-HER3 antibody, anti-ICOS antibody, anti-IL-13 antibody, anti-IL-17 antibody, anti-IL-6 antibody, anti-IL6R antibody, anti-LAG3 antibody, anti-LGR5 antibody, anti-PDL1 antibody, anti-TIM3 antibody, anti-TNF antibody, anti-VEGF antibody, or antibody fragment thereof.

11. The trans-cyclooctene-modified bispecific antibody of claim 10, wherein the bispecific antibody portion is AMF13, NXT007, LY3164530, JNJ-61186372, EMB01, MEHD7945A, RG7221, RG7386, ATN-103, TS-152, ALX-0061, AFM13, AK112, IBI315, ABT122, RO7040547, SAR156597, AZ17, BI 836880, vanucizumab, ABT-165, OMP-305B83, TR009, EMB01, MCLA-158, ZW25, GTB-3550, TG-1801, XmAB23104, AK104, MGD019, XmAb20717, MEDI5752, MGD013, RG7769, LY3434172, FS118, KN046, or LY3415244.

12. The trans-cyclooctene-modified bispecific antibody of any one of claims 1-10, wherein the first antigen target binding moiety is an anti-CD3 antibody or anti-CD3 antibody fragment.

13. The trans-cyclooctene-modified bispecific antibody of claim 12, wherein the second antigen target binding moiety is an anti-B7H3 antibody, anti-BCMA antibody, anti-CD123 antibody, anti-CD19 antibody, anti-CD20 antibody, anti-CD33 antibody, anti-CD38 antibody, anti-CEA antibody, anti-CLEC12A antibody, anti-DLL3 antibody, anti-EGFRvIII antibody, anti-EpCAM antibody, anti-FcRH5 antibody, anti-FLT3 antibody, anti-GD2 antibody, anti-GPA33 antibody, anti-GPC antibody, anti-GPC3 antibody, anti-GPRC5D antibody, anti-HER2 antibody, anti-HIV1 Env antibody, anti-MUC16 antibody, anti-P-cadherin, anti-PSMA antibody, or anti-SSTR2 antibody, or antibody fragment thereof.

14. The trans-cyclooctene-modified bispecific antibody of any preceding claim, wherein the bispecific antibody portion is AFM11, ERY947, RG7802, AMG420, AMG330, AMG212, AMG596, AMG552, AMG673, AMG427, AMG701, MGD006, MGD007, AMG424, MGD011, blinatumomab, AMG562, A-319, RG7828, REGN1979, AR6026, GEN3013, FBTA05, plamotamab, AMG330, AMG673, AMV-564, GEM333, GBR1342, AMG424, AMG420, AMG701, JNJ-64007957, EM801, PF-06863135, REGN5458, TNB-383B, APV0436, MGD006, Xmab14045, JNJ-63709178, MCLA-117, RG6160, AMG427, JNJ-64407564, AMG111, RG7802, solitomab, catumaxomab, pasotuxizumab, HPN-424, AMG160, MOR209, BAY2010112, ertumaxomab, GBR1302, H802, RG6294, PF06671008, MGD007, MGD009, AMG757, REGN4018, AMG596, ERY974, tidutamab, huGD2-BsAB, or MGD014.

15. The trans-cyclooctene-modified bispecific antibody of any preceding claim, wherein the bispecific antibody comprises one or more Fv, Fe, Fab, (Fab′)2, single chain Fv (scFv), single heavy chain antibody (VHH), diabody, triabody, tetrabody, bifunctional hybrid antibody, CDR1, CDR2, CDR3, variable region, framework region, constant region, heavy chain, light chain, alternative scaffold non-antibody molecule (DART, DARPin), or a combination thereof.

16. The trans-cyclooctene-modified bispecific antibody of any preceding claim, wherein the bispecific antibody comprises a scFv.

17. The trans-cyclooctene-modified bispecific antibody of any preceding claim, wherein the bispecific antibody comprises a VHH.

18. The trans-cyclooctene-modified bispecific antibody of any preceding claim, wherein the bispecific antibody comprises at least a portion of a Fab fragment.

19. The trans-cyclooctene-modified bispecific antibody of any preceding claim, wherein the trans-cyclooctene moiety is covalently bonded to the bispecific antibody via a linker.

20. The trans-cyclooctene-modified bispecific antibody of claim 19, wherein the trans-cyclooctene moiety is covalently bonded to the bispecific antibody via a cysteine or lysine residue on the bispecific antibody.

21. The trans-cyclooctene-modified bispecific antibody of any preceding claim, wherein the linker comprises one or more amino acids.

22. The trans-cyclooctene-modified bispecific antibody of any preceding claim, wherein the linker comprises a polypeptide.

23. The trans-cyclooctene-modified bispecific antibody of any preceding claim, wherein the linker comprises one or more of a hydrazone, a hydrazide, a disulfide, a N-succinimidyl-4-(2-pyridyldithio)pentanoate (SPP), a N-succinimidyl-4-(2-pyridyldithio)butyrate (SPDB), a 4-(4′-acetylphenoxy)butanoic acid (AcBut), one or more linear or branched, natural or unnatural amino acid, a valine-citrulline (Val-Cit) moiety, or a phenylalanine-lysine (Phe-Lys) moiety.

24. The trans-cyclooctene-modified bispecific antibody of any preceding claim, wherein the linker comprises 1 to 100 linking atoms, from 1 to 50 linking atoms, or from 5 to 50 linking atoms, or from 10 to 50 linking atoms, or from 1 to 40 linking atoms, or from 1 to 30 linking atoms, or from 1 to 20 linking atoms, or from 1 to 10 linking atoms, or from 1 to 5 linking atoms, or from 5 to 30 linking atoms, or from 10 to 30 linking atoms, or from 5 to 40 linking atoms, or from 5 to 50 linking atoms, or from 10 to 50 linking atoms.

25. The trans-cyclooctene-modified bispecific antibody of any preceding claim, wherein the linker comprises one or more chain heteroatoms and one or more alkylene, alkenylene, alkynylene, arylene, heteroarylene, cycloalkylene or heterocycloalkylene moieties; wherein each alkylene, alkenylene, alkynylene, arylene, heteroarylene, cycloalkylene or heterocycloalkylene moiety, may be independently optionally substituted with one to five substituents independently selected from oxo, halo, C₁₋₄ alkyl, C₁₋₄ alkoxy, and C₁₋₄ haloalkyl.

26. The trans-cyclooctene-modified bispecific antibody of any preceding claim, wherein the linker is an alkylene linker optionally comprising one or more —O—, —S—, amine, ester, amide, carbamate, carbonate, thio-succinimide, or ketone functional group.

27. The trans-cyclooctene-modified bispecific antibody of any preceding claim, wherein L is of the formula:
—Y¹⁰—(CH₂)_{n′}—Y²⁰—(CH₂)_{m″}—Y³⁰—
wherein:
each of Y¹⁰, Y²⁰, and Y³⁰ are independently a bond, —NR¹¹⁰—, —O—, —S(O)₀₋₂—, —NR¹¹⁰C(O)—, —OC(O)NR¹¹⁰—, —C(O)NR¹¹⁰—, —NR¹¹⁰S(O)₂—, —S(O)₂NR¹¹⁰—, —CR¹²⁰═N—NR¹¹⁰—, —NR¹¹⁰—N═CR¹²⁰—, —C(O)—, —OC(O)—, —OC(O)O—, alkylene, alkenylene, alkynylene, arylene, heteroarylene, cycloalkylene, or heterocycloalkylene; wherein each alkylene, alkenylene, alkynylene, arylene, heteroarylene, cycloalkylene, or heterocycloalkylene is independently optionally substituted with one to five substituents independently selected from oxo, halo, C₁₋₄ alkyl, C₁₋₄ alkoxy, and C₁₋₄ haloalkyl;
each R¹¹⁰ is independently hydrogen, C₁₋₄ alkyl, C₁₋₄ haloalkyl, aryl, heteroaryl, cycloalkyl or heterocyclyl;
each R¹²⁰ is independently hydrogen, C₁₋₄ alkyl, C₁₋₄ haloalkyl, aryl, heteroaryl, cycloalkyl or heterocyclyl; and
n′ and m″ are each independently 0, 1, 2, 3, 4, 5, 6, 7, or 8.

28. The trans-cyclooctene-modified bispecific antibody of any preceding claim, wherein the trans-cyclooctene moiety is of Formula X: 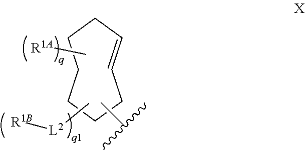
wherein:
R^{1A}, at each occurrence, is independently selected from the group consisting of C₁₋₄alkyl, C₁₋₄haloalkyl, and C₁₋₄alkoxy;
q is 0, 1, or 2;
q1 is 0 or 1;
R^{1B}, at each occurrence, is independently selected from the group consisting of G¹, OH, —NR^{1c}—C₁₋₄alkylene-G¹, —NR^{1c}—C₁₋₄alkylene-N(R^{1d})₂, —NR^{1c}—C₁₋₆alkylene-N(C₁₋₄alkyl)₃⁺, —N(R^{1c})CHR^{1e}CO₂H, —N(R^{1c})—C₁₋₆alkylene-CO₂H, —N(R^{1c})—C₂₋₄alkylene-(N(C₁₋₄alkylene-CO₂H)—C₂₋₄alkylene), —N(C₁₋₄alkylene-CO₂H)₂, —N(R^{1c})CHR^{1e}C(O)OC₁₋₆alkyl, —N(R^{1c})—C₁₋₆alkylene-C(O)OC₁₋₆alkyl, —N(R^{1f})—C₂₋₄alkylene-(N(C₁₋₄alkylene-C(O)OC₁₋₆alkyl)-C₂₋₄alkylene), —N(C₁₋₄alkylene-C(O)OC₁₋₆alkyl)₂, —N(R^{1c})—C₁₋₆alkylene-SO₃H, —N(R^{1c})—(CH₂CH₂O)₁₋₃—CH₂CH₂N((CH₂CH₂O)₁₋₃—C₁₋₆alkylene-CO₂H)₂, and —N(R^{1c})—CH(CH₂O—(CH₂CH₂O)₀₋₂—C₁₋₆alkylene-CO₂H)₂;
R^{1c} and R^{1d}, at each occurrence, are independently hydrogen or C₁₋₄alkyl;
R^{1e}, at each occurrence, is independently —C₁₋₄alkylene-CO₂H, —C₁₋₄alkylene-CONH₂, or —C₁₋₄alkylene-OH;
R^{1f}, at each occurrence, is independently hydrogen, C₁₋₆alkyl, or C₁₋₄alkylene-CO₂H;
n, at each occurrence, is independently 0, 1, 2, or 3;
L², at each occurrence, is independently selected from the group consisting of —C(O)— and C₁₋₃alkylene; and
G¹, at each occurrence, is independently an optionally substituted heterocyclyl.

29. The trans-cyclooctene-modified bispecific antibody of any preceding claim, wherein the trans-cyclooctene moiety is of Formula XI: 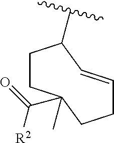
wherein:
R² is —OH, 2-aminoethanesulfonic acid, an N-linked natural or unnatural amino acid, or an optionally substituted ethylenediamine; wherein R² may be optionally further substituted with a polyether.

30. The trans-cyclooctene-modified bispecific antibody of any preceding claim, wherein the trans-cyclooctene moiety is of Formula:

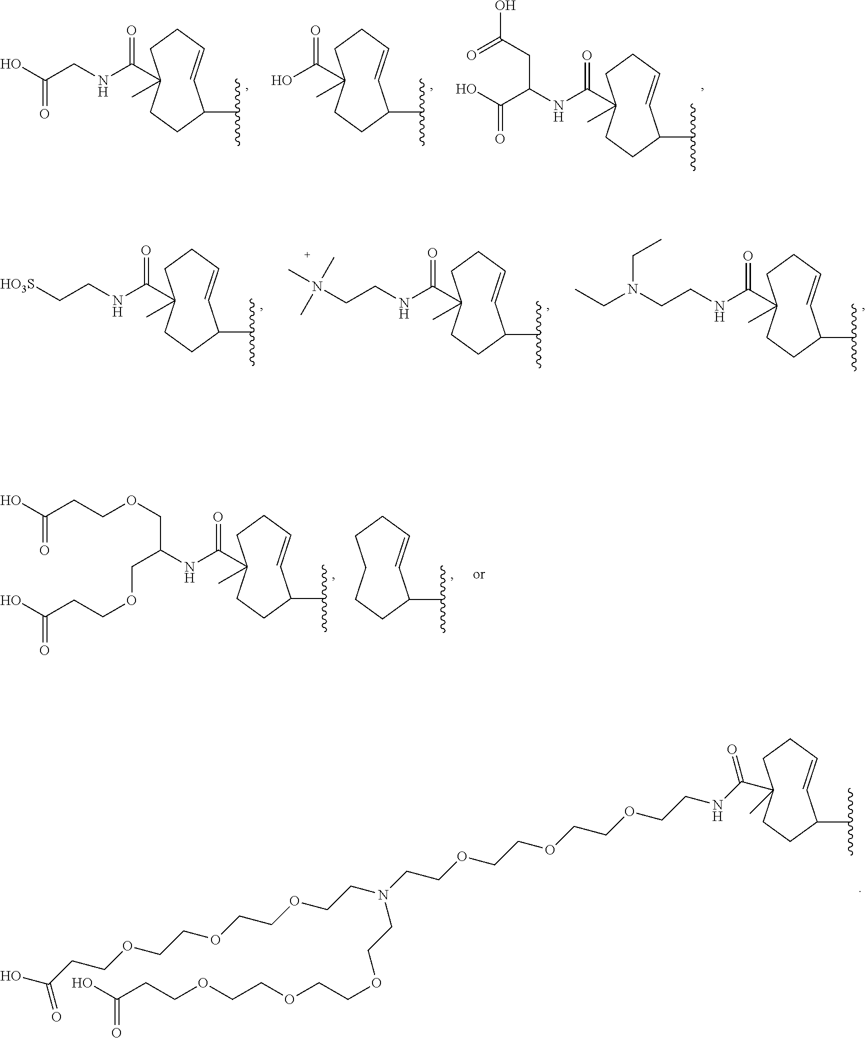

31. A system comprising the trans-cyclooctene-modified bispecific antibody of any preceding claim and a tetrazine activator.

32. The system of claim 31, wherein the tetrazine activator is formulated for systemic administration.

33. The system of claim 31 or 32, wherein the tetrazine activator is of Formula IV: 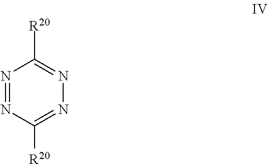
wherein:
each R²⁰ is independently selected from the group consisting of hydrogen, halo, cyano, nitro, alkyl, alkenyl, alkynyl, haloalkyl, heteroalkyl, aryl, heteroaryl, heterocyclyl, cycloalkyl, OR′, SR′, C(═O)R′, C(═S)R′, OC(═O)R′″, SC(═O)R″, OC(═S)R′″, SC(═S)R′″, S(═O)R′, S(═O)₂R′″, S(═O)₂NR′R″, C(═O)O—R′, C(═O)S—R′, C(═S)OR′, C(═S)SR′, C(═O)NR′R″, C(═S)NR′R″, NR′R″, NR′C(═O)R″, NR′C(═S)R″, NR′C(═O)OR″, NR′C(═S)OR″, NR′C(═O)SR″, NR′C(═S)SR″, OC(═O)NR′R″, SC(═O)NR′R″, OC(═S)R′R′″, SC(═S)R′R″, NR′C(═O)NR″R″, and NR′C(═S)NR″R″; wherein each alkyl, alkenyl, alkynyl, haloalkyl, heteroalkyl, aryl, heteroaryl, heterocyclyl, or cycloalkyl is optionally substituted with one to three Z′;
each Z′ is independently selected from halo, oxo, cyano, nitro, hydroxy, alkyl, haloalkyl, alkenyl, alkynyl, alkoxy, haloalkoxy, heteroalkyl, aryl, heteroaryl, heterocyclyl, cycloalkyl, OR′, SR′, C(═O)R′, C(═S)R′, OC(═O)R′″, SC(═O)R″, OC(═S)R′″, SC(═S)R′″, S(═O)R′, S(═O)₂R′″, S(═O)₂NR′R″, C(═O)O—R′, C(═O)S—R′, C(═S)O—R′, C(═S)S—R′, C(═O)NR′R″, C(═S)NR′R″, NR′R″, NR′C(═O)R″, NR′C(═S)R″, NR′C(═O)OR″, NR′C(═S)OR″, NR′C(═O)SR″, NR′C(═S)SR″, OC(═O)NR′R″, SC(═O)NR′R″, OC(═S)R′R′″, SC(═S)R′R″, NR′C(═O)NR″R″, and NR′C(═S)NR″R″;
R′ and R″, at each occurrence, are independently selected from hydrogen, aryl, and alkyl; and
R′″, at each occurrence, is independently selected from aryl and alkyl.

34. The system of claim 33, wherein each R²⁰ is independently phenyl, pyrimidinyl, triazinyl, oxazolyl, isoxazole, imidazolyl, oxadiazolyl, 6,7-dihydro-5H-pyrrolo[3,4-d]pyrimidinyl, 5,6,7,8-tetrahydropyrido[4,3-d]pyrimidinyl, or 5,6,7,8-tetrahydropyrido[3,4-d]pyrimidinyl; wherein is independently optionally substituted with one to three Z¹.

35. The system of claim 31-34, wherein the tetrazine activator is a therapeutic support composition comprising a biocompatible support and a tetrazine-containing group.

36. The system of claim 35, wherein the therapeutic support composition comprising a biocompatible support and a tetrazine-containing group of Formula IVA, IVB, or IVC: 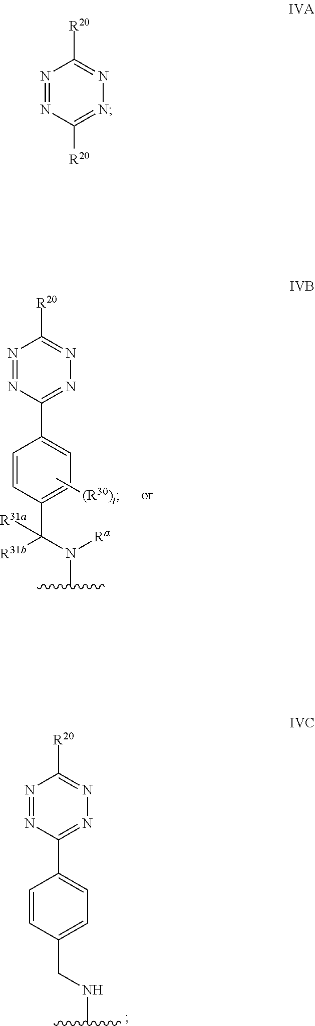
wherein:
R²⁰ is selected from the group consisting of hydrogen, halogen, cyano, nitro, alkyl, alkenyl, alkynyl, heteroalkyl, aryl, heteroaryl, heterocycle, cycloalkyl, cycloalkenyl, CF₃, CF₂—R′, NO₂, OR′, SR′, C(═O)R′, C(═S)R′, OC(═O)R′″, SC(═O)R″, OC(═S)R′″, SC(═S)R′″, S(═O)R′, S(═O)₂R′″, S(═O)₂NR′R″, C(═O)O—R′, C(═O)S—R′, C(═S)O—R′, C(═S)S—R′, C(═O)NR′R″, C(═S)NR′R″, NR′R″, NR′C(═O)R″, NR′C(═S)R″, NR′C(═O)OR″, NR′C(═S)OR″, NR′C(═O)SR″, NR′C(═S)SR″, OC(═O)NR′R″, SC(═O)NR′R″, OC(═S) R′R′″, SC(═S)R′R″, NR′C(═O)NR″R″, and NR′C(═S)NR″R″;
R′ and R″ at each occurrence are independently selected from hydrogen, aryl and alkyl;
R′″ at each occurrence is independently selected from aryl and alkyl;
R³⁰ is halogen, cyano, nitro, hydroxy, alkyl, haloalkyl; alkenyl, alkynyl, alkoxy; haloalkoxy;
heteroalkyl, aryl, heteroaryl, heterocycle, cycloalkyl, or cycloalkenyl;
R^{a}, R^{31a} and R^{31b} are each independently hydrogen, C₁-C₆-alkyl, or C₁-C₆-haloalkyl; and
t is 0, 1, 2, 3, or 4.

37. The system of claim 31, wherein the tetrazine activator is of Formula V: 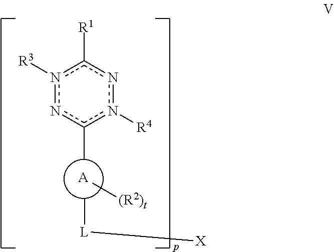
wherein:
ring A is aryl, cycloalkyl, heterocyclyl, or heteroaryl;
the dotted lines represent additional bonds to form a tetrazine when R³ and R⁴ are both absent, or a dihydrotetrazine when R³ and R⁴ are both present; provided that when ring A is aryl, then R³ and R⁴ are both present;
X is a biocompatible support, antibody, or antibody fragment moiety;
p is 1-150;
L, at each occurrence, is independently a linker;
R¹, at each occurrence, is independently selected from the group consisting of hydrogen, halo, cyano, nitro, alkyl, alkenyl, alkynyl, haloalkyl, heteroalkyl, aryl, heteroaryl, heterocyclyl, cycloalkyl, OR′, SR′, C(═O)R′, C(═S)R′, OC(═O)R′″, SC(═O)R″, OC(═S)R′″, SC(═S)R′″, S(═O)R′, S(═O)₂R′″, S(═O)₂NR′R″, C(═O)O—R′, C(═O)S—R′, C(═S)OR′, C(═S)SR′, C(═O)NR′R″, C(═S)NR′R″, NR′R″, NR′C(═O)R″, NR′C(═S)R″, NR′C(═O)OR″, NR′C(═S)OR″, NR′C(═O)SR″, NR′C(═S)SR″, OC(═O)NR′R″, SC(═O)NR′R″, OC(═S)R′R′″, SC(═S)R′R″, NR′C(═O)NR″R″, and NR′C(═S)NR″R″; wherein each alkyl, alkenyl, alkynyl, haloalkyl, heteroalkyl, aryl, heteroaryl, heterocyclyl, or cycloalkyl is optionally substituted with one to three Z¹;
R², at each occurrence, is independently halo, cyano, nitro, hydroxy, alkyl, haloalkyl, alkenyl, alkynyl, alkoxy, haloalkoxy, heteroalkyl, aryl, heteroaryl, heterocyclyl, cycloalkyl, —C(═O)-alkyl, —C(═O)-haloalkyl, —C(═O)-alkenyl, —C(═O)-alkynyl, —C(═O)-alkoxy, —C(═O)-haloalkoxy, —C(═O)-heteroalkyl, —C(═O)-aryl, —C(═O)-heteroaryl, —C(═O)-heterocyclyl, or —C(═O)-cycloalkyl; wherein each alkyl, haloalkyl, alkenyl, alkynyl, alkoxy, haloalkoxy, heteroalkyl, aryl, heteroaryl, heterocyclyl, or cycloalkyl is optionally substituted with one to three Z¹;
R³ and R⁴ are both absent; or R³ and R⁴ are each independently hydrogen or a group capable of being removed after a triggering event;
each Z¹ is independently selected from halo, oxo, cyano, nitro, hydroxy, alkyl, haloalkyl, alkenyl, alkynyl, alkoxy, haloalkoxy, heteroalkyl, aryl, heteroaryl, heterocyclyl, cycloalkyl, OR′, SR′, C(═O)R′, C(═S)R′, OC(═O)R′″, SC(═O)R″, OC(═S)R′″, SC(═S)R′″, S(═O)R′, S(═O)₂R′″, S(═O)₂NR′R″, C(═O)O—R′, C(═O)S—R′, C(═S)O—R′, C(═S)S—R′, C(═O)NR′R″, C(═S)NR′R″, NR′R″, NR′C(═O)R″, NR′C(═S)R″, NR′C(═O)OR″, NR′C(═S)OR″, NR′C(═O)SR″, NR′C(═S)SR″, OC(═O)NR′R″, SC(═O)NR′R″, OC(═S)R′R′″, SC(═S)R′R″, NR′C(═O)NR″R″, and NR′C(═S)NR″R″;
R′ and R″, at each occurrence, are independently selected from hydrogen, aryl, and alkyl;
R′″, at each occurrence, is independently selected from aryl and alkyl; and
t, at each occurrence, is independently is 0, 1, 2, 3, or 4.

38. The system of claim 34, wherein the biocompatible support is a polymer, viscous or non-viscous liquid material, gel, hydrogel, polysaccharide hydrogel, a cross-linked polymer matrix, a metal, a ceramic, a plastic, a bone graft material, alginate, cellulose, chitosan, hyaluronic acid, chondroitin sulfate, heparin, particle, nanoparticle, microparticle, alginate, cellulose, hyaluronic acid, chitosan, chitosin, chitin, hyaluronic acid, or chondroitin sulfate.

39. The system of any one of claims 35-38, wherein the tetrazine-containing group is bonded to the biocompatible support via a linker.

40. The system of claim 31, 32, or 37, wherein the tetrazine activator comprises an antibody or antibody fragment moiety.

41. The system of claim 40, wherein the tetrazine activator is of Formula I: 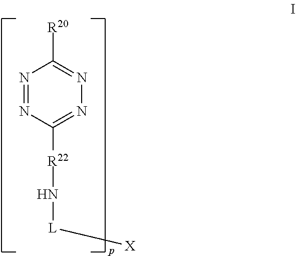
wherein:
X is an antibody or antibody fragment moiety;
p is 1-16;
L, at each occurrence, is independently a linker;
R²⁰, at each occurrence, is independently selected from the group consisting of hydrogen, halogen, cyano, nitro, alkyl, alkenyl, alkynyl, heteroalkyl, aryl, heteroaryl, heterocyclyl, cycloalkyl, cycloalkenyl, CF₃, CF₂—R′, NO₂, OR′, SR′, C(═O)R′, C(═S)R′, OC(═O)R′″, SC(═O)R″, OC(═S)R′″, SC(═S)R′″, S(═O)R′, S(═O)₂R′″, S(═O)₂NR′R″, C(═O)O—R′, C(═O)S—R′, C(═S)O—R′, C(═S)S—R′, C(═O)NR′R″, C(═S)NR′R″, NR′R″, NR′C(═O)R″, NR′C(═S)R″, NR′C(═O)OR″, NR′C(═S)OR″, NR′C(═O)SR″, NR′C(═S)SR″, OC(═O)NR′R″, SC(═O)NR′R″, OC(═S)R′R′″, SC(═S)R′R″, NR′C(═O)NR″R″, and NR′C(═S)NR″R″;
R²², at each occurrence, is independently a linker of 1 to 100 linking atoms optionally comprising one or more ethylene-oxy, amine, ester, amide, carbamate, carbonate, or ketone functional group;
R′ and R″, at each occurrence, are independently selected from hydrogen, aryl, and alkyl; and
R′″ at each occurrence is independently selected from aryl and alkyl.

42. The system of claim 40, wherein the tetrazine activator is of Formula II: 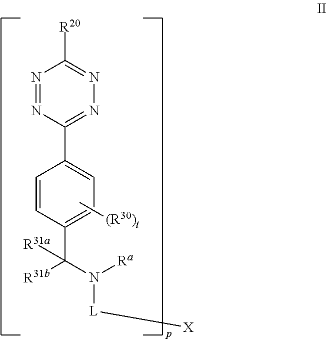
wherein:
X is an antibody or antibody fragment moiety;
p is 1-16;
L, at each occurrence, is independently a linker;
R²⁰, at each occurrence, is independently selected from the group consisting of hydrogen, halogen, cyano, nitro, alkyl, alkenyl, alkynyl, heteroalkyl, aryl, heteroaryl, heterocyclyl, cycloalkyl, cycloalkenyl, CF₃, CF₂—R′, NO₂, OR′, SR′, C(═O)R′, C(═S)R′, OC(═O)R′″, SC(═O)R″, OC(═S)R′″, SC(═S)R′″, S(═O)R′, S(═O)₂R′″, S(═O)₂NR′R″, C(═O)O—R′, C(═O)S—R′, C(═S)O—R′, C(═S)S—R′, C(═O)NR′R″, C(═S)NR′R″, NR′R″, NR′C(═O)R″, NR′C(═S)R″, NR′C(═O)OR″, NR′C(═S)OR″, NR′C(═O)SR″, NR′C(═S)SR″, OC(═O)NR′R″, SC(═O)NR′R″, OC(═S)R′R′″, SC(═S)R′R″, NR′C(═O)NR″R″, and NR′C(═S)NR″R″;
R³⁰, at each occurrence, is independently halogen, cyano, nitro, hydroxy, alkyl, haloalkyl, alkenyl, alkynyl, alkoxy, haloalkoxy, heteroalkyl, aryl, heteroaryl, heterocyclyl, cycloalkyl, or cycloalkenyl;
R^{a}, R^{31a} and R^{31b} are each independently hydrogen, C₁-C₆-alkyl, or C₁-C₆-haloalkyl;
R′ and R″, at each occurrence, are independently selected from hydrogen, aryl, and alkyl;
R′″, at each occurrence, is independently selected from aryl and alkyl; and
t is independently is 0, 1, 2, 3, or 4.

43. A method of treating cancer or enhancing or eliciting an immune response, the method comprising administering to a subject in need thereof, a therapeutically effective amount of the trans-cyclooctene-modified bispecific antibody of any of claims 1-34, or the system of any one of claims 35-42.

44. The method of claim 43, wherein the cancer is a melanoma, renal cancer, prostate cancer, ovarian cancer, endometrial carcinoma, breast cancer, glioblastoma, lung cancer, soft tissue sarcoma, fibrosarcoma, osteosarcoma, pancreatic cancer, gastric carcinoma, squamous cell carcinoma of head/neck, anal/vulvar carcinoma, esophageal carcinoma, pancreatic adenocarcinoma, cervical carcinoma, hepatocellular carcinoma, Kaposi's sarcoma, non-Hodgkin's lymphoma, Hodgkins lymphoma, Wilm's tumor/neuroblastoma, bladder cancer, thyroid adenocarcinoma, pancreatic neuroendocrine tumors, Prostatic adenocarcinoma, Nasopharyngeal carcinoma, or cutaneous T-cell lymphoma.

45. The method of claim 43, wherein the cancer is a solid tumor.

46. The method of claim 43, wherein the cancer is a soft tissue sarcoma.

47. The method of claim 43, wherein the cancer is a hematological malignancy.

48. The method of claim 47, wherein the hematological malignancy is myelodysplastic syndrome, acute myeloid leukemia, myelodysplastic syndromes, chronic myelogenous leukemia, chronic myelomonocytic leukemia, primary myelofibrosis, diffuse large B-cell lymphoma, chronic lymphocytic leukemia, monoclonal gammopathy, plasma cell myeloma, follicular lymphoma, marginal zone lymphoma, classical Hodgkin lymphoma, monoclonal B-cell lymphocytosis, lymphoproliferative disorder NOS, T-cell lymphoma, precursor B-lymphoblastic leukemia, mantle cell lymphoma, plasmacytoma, Burkitt lymphoma, T-cell leukemia, hairy-cell leukemia, precursor T-lymphoblastic leukemia, or nodular lymphocyte predominant Hodgkin lymphoma.

49. The method of claim 48, wherein the immune response is an increase in one or more of leukocytes, lymphocytes, monocytes, and eosinophils.
